(19) United States
(12) Patent Application Publication
(10) Pub. No.: US 2026/0015387 A1
(43) Pub. Date: Jan. 15, 2026

(54) MITOCHONDRIAL ANTIOXIDANT PEPTIDES

(71) Applicants: University of Vermont and State Agricultural College, Burlington, VT (US); National Jewish Health, Denver, CO (US)

(72) Inventors: Robert J. HONDAL, Burlington, VT (US); Kaelyn JENNY, Essex Junction, VT (US); Brian DAY, Englewood, CO (US)

(21) Appl. No.: 18/992,504

(22) PCT Filed: Jul. 10, 2023
(86) PCT No.: PCT/US2023/069907

§ 371 Date: Jan. 8, 2025

Related U.S. Application Data
(60) Provisional application No. 63/368,038, filed on Jul. 8, 2022.
(60) Provisional application No. 63/482,793, filed on Feb. 1, 2023.

(51) Int. Cl. C07K 5/11; A61K 9/19; A61K 38/00

(52) U.S. Cl. CPC C07K 5/1019; A61K 9/19; A61K 38/00

(57) ABSTRACT

Provided are proteins and peptides including the sequence -RXKF-, where X is a 2-thiohistidine residue or 2-thiohistidine residue analogue and each residue of the sequence has D or L stereochemistry. Also provided are compositions and methods of using these proteins and peptides. The protein and peptides may be used to treat individuals having diseases associated with mitochondrial dysfunction and/or diseases associated with HOCl-mediated injury and/or chlorine gas exposure.

### CROSS REFERENCE TO RELATED APPLICATIONS

This application claims priority to U.S. Provisional Application No. 63/368,038, filed Jul. 8, 2022, and U.S. Provisional Application No. 63/482,793, filed Feb. 1, 2023, the disclosures of which are hereby incorporated by reference.

### SEQUENCE LISTING

The instant application contains a Sequence Listing, which has been submitted in .xml format and is hereby incorporated by reference in its entirety. Said .xml copy was created on Jul. 10, 2023, is named “073777_00120_ST26.xml”, and is 2,644 bytes in size.

### STATEMENT REGARDING FEDERALLY SPONSORED RESEARCH

This invention was made with government support under grant no. HL141146 awarded by the National Institutes of Health. The government has certain rights in the invention.

### BACKGROUND OF THE DISCLOSURE

Szeto-Schiller peptides (SS-peptides) were developed as an outgrowth of research of the DALDA peptide, which is a μ-opioid agonist peptide. It was found that replacing the tyrosine residue of DALDA with 2,-6-dimethyltyrosine (Dmt) significantly increased the binding affinity of the peptide for the receptor due to the increased hydrophobicity of Dmt compared to Tyr. It was further discovered the Dmt-DALDA could be transported across cellular membranes. Based on the antioxidant properties of Dmt, which is a type of vitamin E analogue, and the transport properties of Dmt-DALDA, a new set of Dmt-containing peptides were designed that targeted the inner membrane of the mitochondria. These peptides were named “SS-peptides” after their inventors, Hazel Szeto and Peter Schiller. These peptides were designed to target the inner membrane of the mitochondria to act as antioxidants to protect cardiolipin from being oxidized. The oxidation of cardiolipin, an abundant lipid in the inner membrane, is the key initiating event in apoptosis. When the unsaturated fatty acids of cardiolipin are oxidized, cytochrome c is liberated from the inner membrane and then is translocated from the mitochondria through the mitochondrial permeability transition pore, which then initiates formation of the apoptopsome. It was found that SS-31, a peptide of sequence H-D-Arg-Dmt-Lys-Phe-NH₂, was a particular effective mitochondrial-targeted antioxidant that prevented the oxidation of linoleic acid (an unsaturated fatty acid attached to cardiolipin), scavenged cellular reactive oxygen species caused by the introduction of 1-butyl hydroperoxide, and prevented reperfusion-associated myocardial stunning after global ischemia.

### BRIEF DESCRIPTION OF THE FIGURES

For a fuller understanding of the nature and objects of the disclosure, reference should be made to the following detailed description taken in conjunction with the accompanying figures.

FIG. 1 shows (left) a known Schiller-Szeto peptide, (middle) a peptide of the present disclosure; and right; and (right) a histidine control of the middle peptide. The bottom shows the structure of vitamin E. SS-31 peptide is described above, analogue with 2TH is -RXKF-, and control is SEQ ID NO:1 (RHKF).

FIG. 2 shows the structure of cardiolipin and DPPH.

FIG. 3 shows quenching experiments of the various peptides with DPPH.

FIG. 4 shows (A) quenching experiments of the various peptides with ABTS. (B) shows a zoom in of (A). This is a comparison of the WT SS-31 peptide with our 2TH-analogue. The WT quenches the ABTS radical better. The WT is slightly better in this assay.

FIG. 5 shows the histidine control does not quench the ABTS radical.

FIG. 6 shows a comparison of the ABTS scavenging ability of the three peptides.

FIG. 7 shows the data of an EPR assay to measure the ability of the peptides to quench hydroxyl radicals.

FIG. 8 shows the data of an ROS glow assay.

FIG. 9 shows data of an HOCl protection assay using 16HBE41o-cells.

FIG. 10 shows the data of a rotenone cell viability assay.

FIG. 11 shows the data of a rotenone cell viability assay.

FIG. 12 shows the data of a rotenone cell viability assay.

FIG. 13 shows the data of a rotenone cell viability assay.

FIG. 14 shows the data of a rotenone cell viability assay.

FIG. 15 shows the data of a rotenone cell viability assay.

FIG. 16 shows a mass spectrum of the 2TH-containing peptide of the present disclosure. Sequence shown is -RXKF-, where X is 2TH.

FIG. 17 shows that the amino acid analogue of ergothioneine, 2-thiohistidine, can be inserted into a peptide and imparts a gain of antioxidant function and metal binding ability.

FIG. 18 shows data for a binding assay. Cardiolipin binds to cytochrome c, preventing it from being reduced by ascorbate. When the peptide binds cardiolipin, cytochrome c is free to be reduced by ascorbate. The data shows that our analogue binds to CL equally well as SS31.

FIG. 19 shows the peptides of the present disclosure display protection against HOCl-mediated cell death. 16HBE41o-cells were treated with 200 μM of HOCl for 60 min in PBS with or without increasing concentrations of peptides. Cells were washed and then placed back in media and cell viability was assess at 24 hours using the MTT assay. % Protection was determined by normalizing data to cell viability with and without oxidant.

FIG. 20 shows the peptides of the present disclosure display protection against Cl₂ gas. 16HBE41o-cells were treated with Cl₂ gas at a concentration of 400 ppm for 15 minutes at various concentrations of peptide. Cell survival was measured by MTT assay.

FIG. 21 shows 2-thioHis is an analogue of ergotioneine (EGT).

FIG. 22 shows comparison of histidine to 2-thioHis.

FIG. 23 shows that EGT has properties similar to His and Cys.

FIG. 24 shows the oxidation chemistry of EGT.

FIG. 25 shows 2-thioHis in a peptide.

FIG. 26 shows the incorporation of 2-thioHis into a peptide. Shown is the lyophilized peptide. SEQ ID NO:2 is shown (HGPLGPL).

FIG. 27 shows 2-thioHis containing peptides are better antioxidants relative to His containing peptides.

FIG. 28 shows potential applications of 2-thioHis containing peptides. The alkyl chains having N and C labels represent peptide backbones.

FIG. 29 shows potential applications of 2-thioHis containing peptides.

FIG. 30 shows a cartoon for an assay of cell protection against HOCl using a 2-thioHis peptide.

FIG. 31 shows comparative data of 2-thioHis peptides and histidine containing peptides for protection against HOCl-mediated cell death.

FIG. 32 shows Thioredoxin Reductase (TrxR) recycles oxidized forms of vitamin C.

FIG. 33 shows the potential for ergothioneine as a substrate for TrxR.

FIG. 34 shows data from a TrxR assay of ESSE.

FIG. 35 shows selenium and ergothioneine in the TrxR cycle.

FIG. 36 shows a reaction involving selenium and vitamin E.

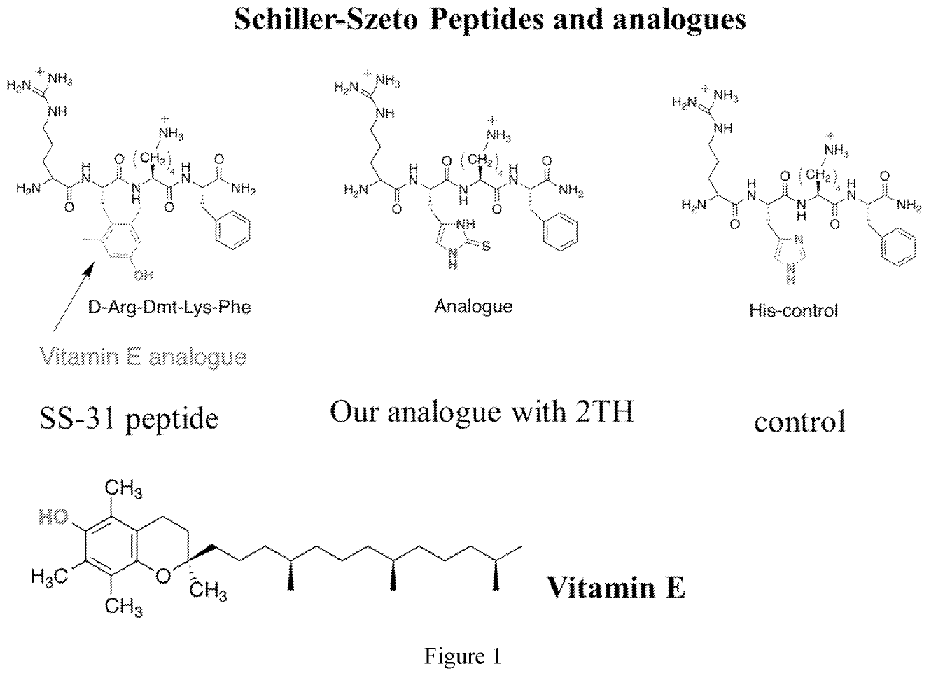

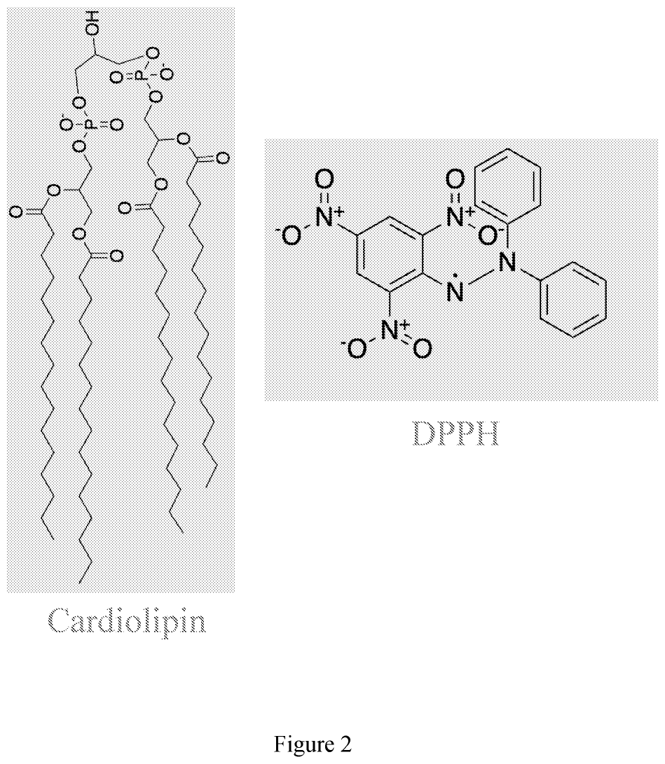

### DETAILED DESCRIPTION OF THE DISCLOSURE

Although claimed subject matter will be described in terms of certain embodiments/examples, other embodiments/examples, including embodiments/examples that do not provide all of the benefits and features set forth herein, are also within the scope of this disclosure. Various structural, logical, process and step changes may be made without departing from the scope of the disclosure.

All ranges provided herein include all values that fall within the ranges to the tenth decimal place, unless indicated otherwise.

In this application, the use of the singular form encompasses the plural and vice versa.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

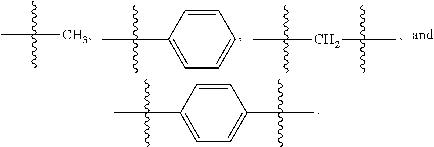

Amino acids may be referred to herein by either their commonly known three letter symbols or by the one-letter symbols recommended by the IUPAC-IUB Biochemical Nomenclature Commission.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. In an example, about refers to ±1%, ±2%, ±3%, ±4%, ±5%, ±6%, ±7%, ±8%, ±9%, ±10%, ±15%, or ±20%.

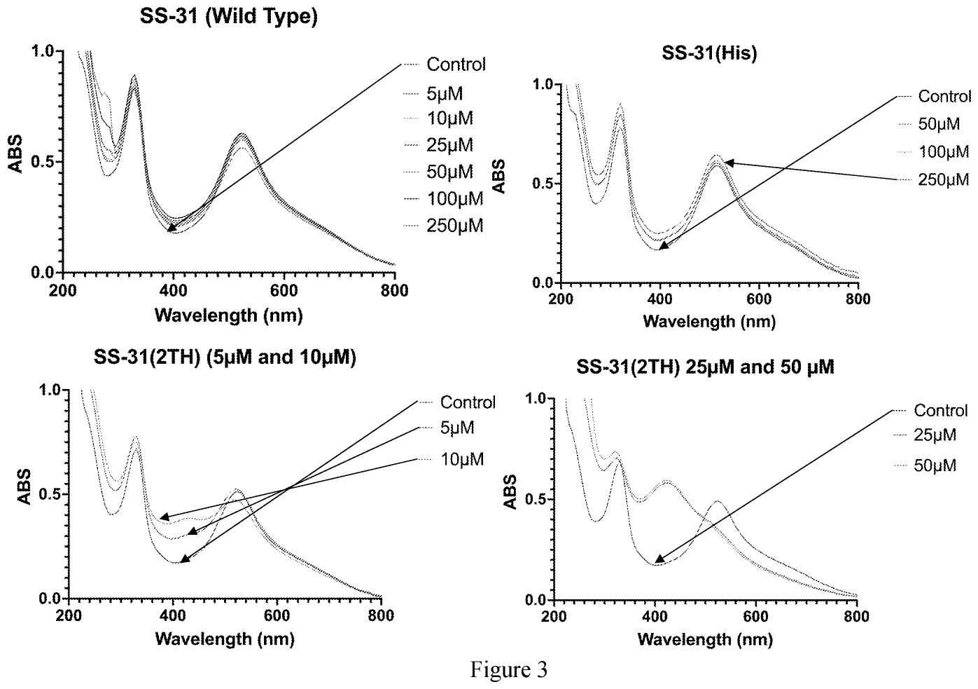

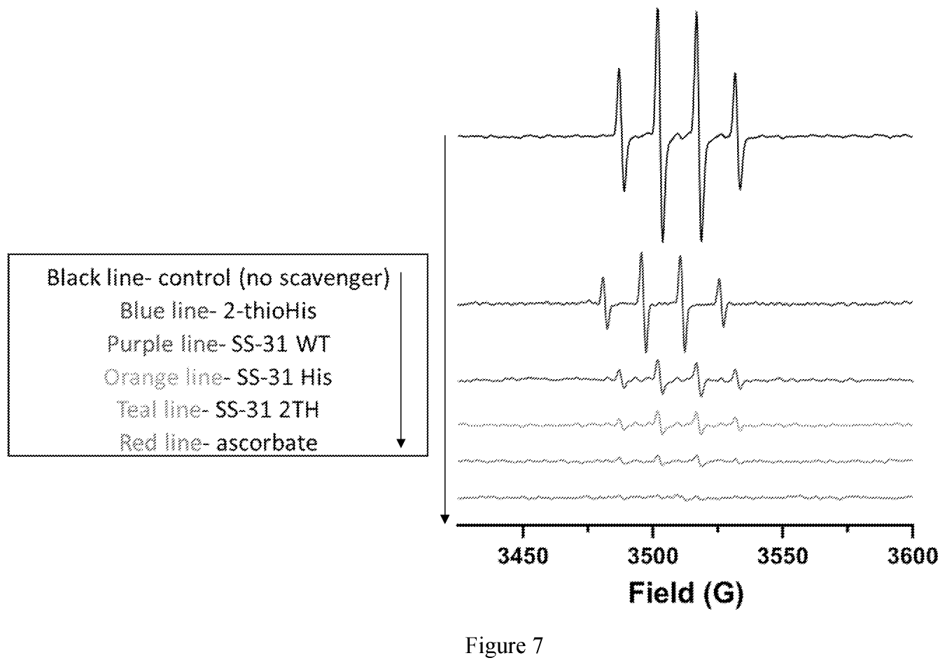

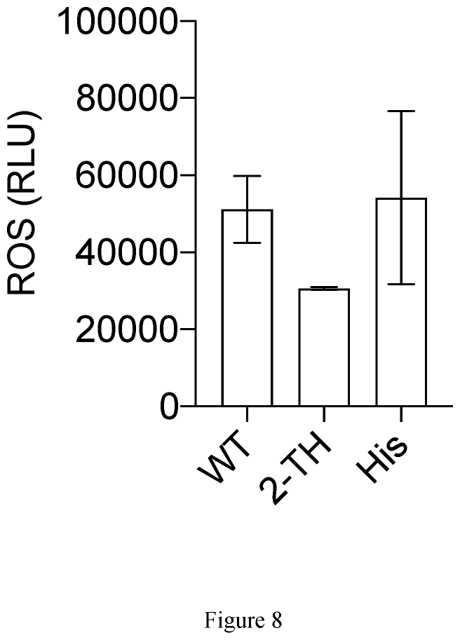

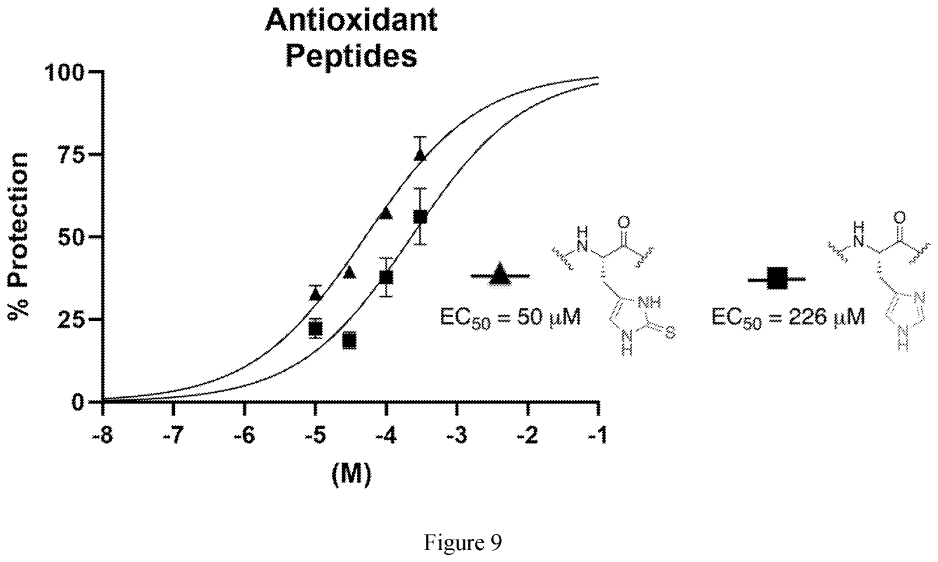

The present disclosure provides an improvement of the SS-31 peptides because it replaces the antioxidant Dmt residue of the peptide with 2-thiohistidine, the amino acid analogue of ergothioneine. The amino acid 2-thiohistidine (2TH) is a powerful antioxidant that is a better scavenger of hypochlorous acid (HOCl)-mediated injury. For example, HOCl is produced in inflammatory diseases of the lung by neutrophils. These data shows that the analogue peptide in which Dmt is replaced with 2TH protects strongly against HOCl-mediated cell death after the cells were treated with 200 μM HOCl, and displayed an EC₅₀ of 50 μM (see FIG. 9). The original SS-31 peptide did not protect against HOCl-mediated injury at all. This represents a clear advantage of 2TH-containing analogues. In addition, the 2TH-containing analogue was better at scavenging the DPPH radical (FIG. 3) and the hydroxyl radical (FIG. 7) compared to the SS-31 peptide. In addition, results of the ROS glow assay (Promega) show that the cellular levels of H₂O₂ were significantly lower compared to the SS-31 peptide (FIG. 8). The sum of the results demonstrate that the 2TH-containing analogue is a better antioxidant in these assays compared to the original SS-31 peptide. This is especially true for HOCl-mediated cellular injury.

In an aspect, the present disclosure provides peptide or protein comprising the following sequence: -RXKF-, where X is a 2TH residue or a 2TH analogue residue and each residue is D or L. 2TH has the following structure:

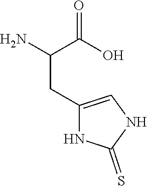 [SMILES: NC(Cc1c[nH]c(=S)[nH]1)C(=O)O]

(stereochemistry is not shown; however 2TH may have D or L stereochemistry). Analogues of 2TH include, but are not limited to:

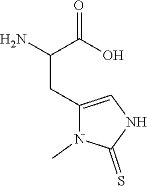

(the residue is

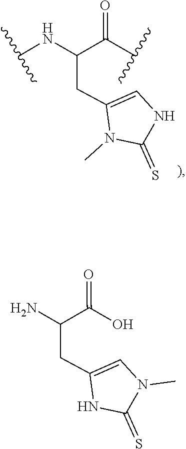

(the residue is

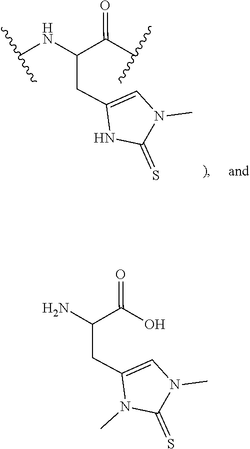

(the residue is

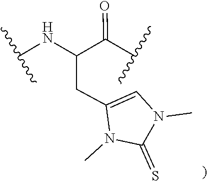 [SMILES: *NC(Cc1cn(C)c(=S)n1C)C(*)=O]

(stereochemistry is not shown for the 2TH analogues; however, 2TH analogues can have D or L stereochemistry). Examples of sequences of the present disclosure, which are derivatives of -RXKF-, include:

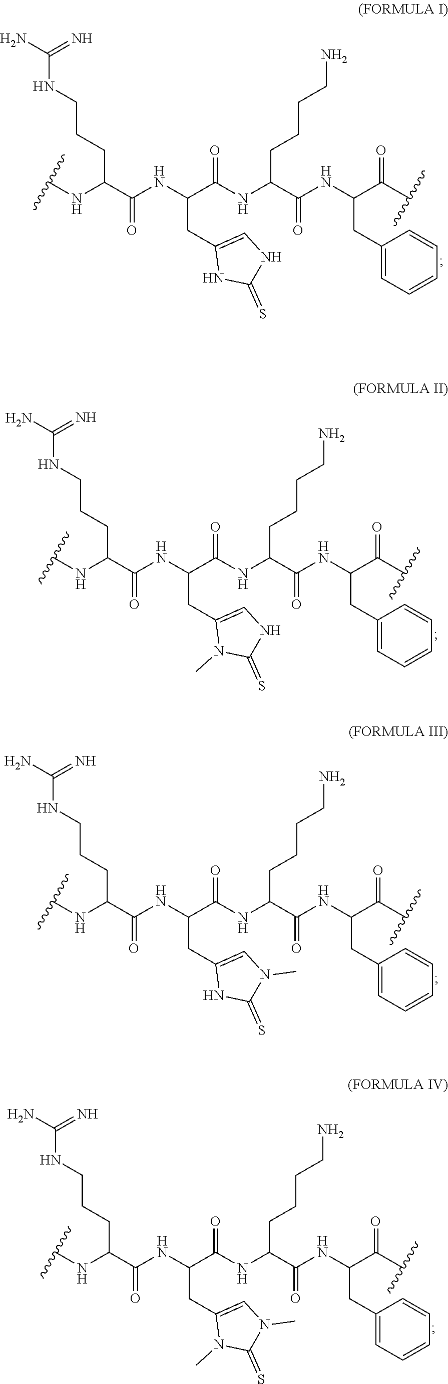 [SMILES: *NC(CCCNC(=N)N)C(=O)NC(Cc1c[nH]c(=S)[nH]1)C(=O)NC(CCCCN)C(=O)NC(Cc1ccccc1)C(*)=O ; *NC(CCCNC(=N)N)C(=O)NC(Cc1c[nH]c(=S)n1C)C(=O)NC(CCCCN)C(=O)NC(Cc1ccccc1)C(*)=O ; *NC(CCCNC(=N)N)C(=O)NC(Cc1cn(C)c(=S)[nH]1)C(=O)NC(CCCCN)C(=O)NC(Cc1ccccc1)C(*)=O ; *NC(CCCNC(=N)N)C(=O)NC(Cc1cn(C)c(=S)n1C)C(=O)NC(CCCCN)C(=O)NC(Cc1ccccc1)C(*)=O]

or protonated forms thereof. The peptides of the present disclosure may be prepared via solid phase peptide synthesis (SPPS) using fluorenylmethoxycarbonyl-based (Fmoc-based) chemistries. Also provided are salts of the present peptides or proteins. The salts may be salts formed from purification and lyophilization. For example, the salt of a peptide or protein may be a TFA salt.

In various embodiments, FORMULAS I to IV may be attached at either its C-terminus or N-terminus to additional amino acid residues or to a larger protein or peptide. For example, the peptide or protein comprises or has the following sequence:

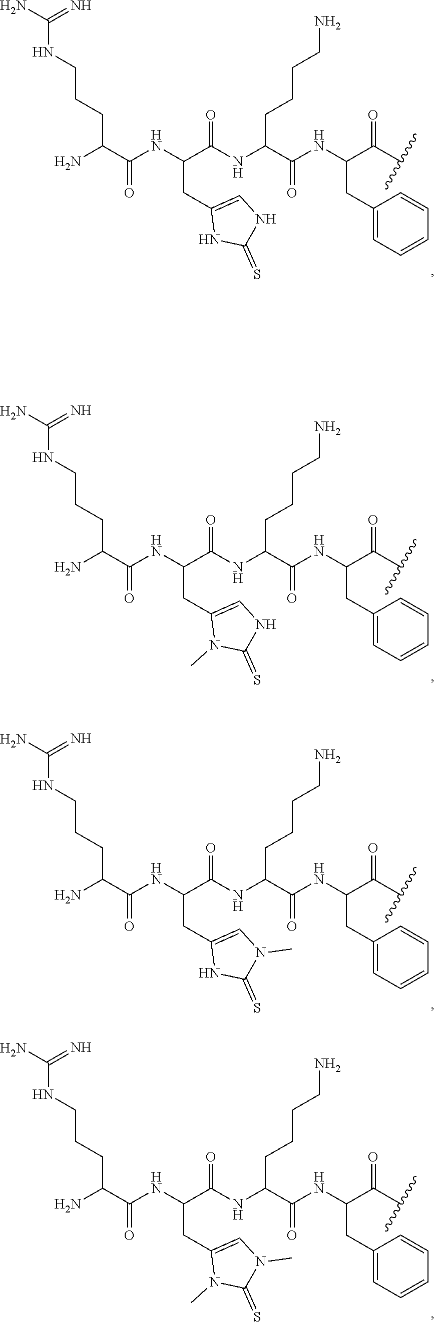 [SMILES: *C(=O)C(Cc1ccccc1)NC(=O)C(CCCCN)NC(=O)C(Cc1c[nH]c(=S)[nH]1)NC(=O)C(N)CCCNC(=N)N ; *C(=O)C(Cc1ccccc1)NC(=O)C(CCCCN)NC(=O)C(Cc1c[nH]c(=S)n1C)NC(=O)C(N)CCCNC(=N)N ; *C(=O)C(Cc1ccccc1)NC(=O)C(CCCCN)NC(=O)C(Cc1cn(C)c(=S)[nH]1)NC(=O)C(N)CCCNC(=N)N ; *C(=O)C(Cc1ccccc1)NC(=O)C(CCCCN)NC(=O)C(Cc1cn(C)c(=S)n1C)NC(=O)C(N)CCCNC(=N)N]

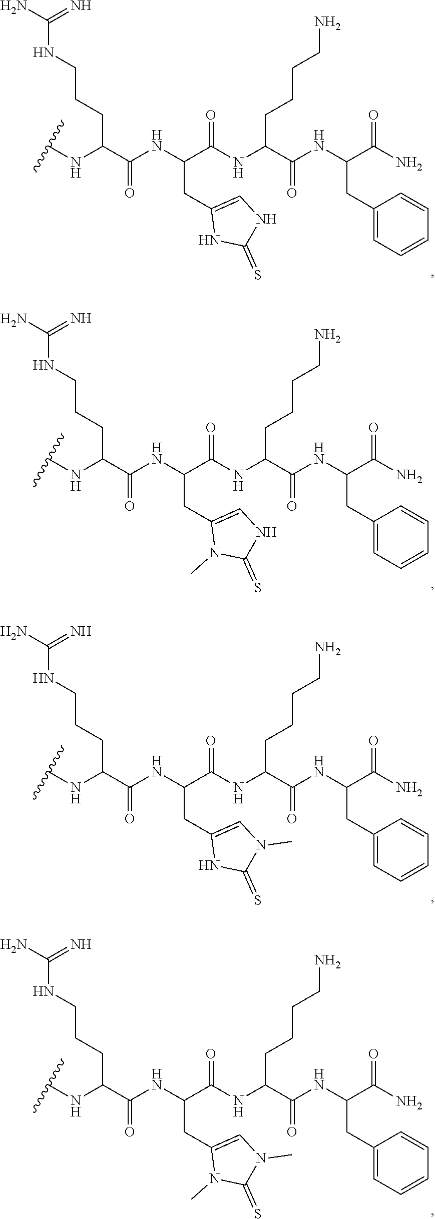 [SMILES: *NC(CCCNC(=N)N)C(=O)NC(Cc1c[nH]c(=S)[nH]1)C(=O)NC(CCCCN)C(=O)NC(Cc1ccccc1)C(N)=O ; *NC(CCCNC(=N)N)C(=O)NC(Cc1c[nH]c(=S)n1C)C(=O)NC(CCCCN)C(=O)NC(Cc1ccccc1)C(N)=O ; *NC(CCCNC(=N)N)C(=O)NC(Cc1cn(C)c(=S)[nH]1)C(=O)NC(CCCCN)C(=O)NC(Cc1ccccc1)C(N)=O ; *NC(CCCNC(=N)N)C(=O)NC(Cc1cn(C)c(=S)n1C)C(=O)NC(CCCCN)C(=O)NC(Cc1ccccc1)C(N)=O]

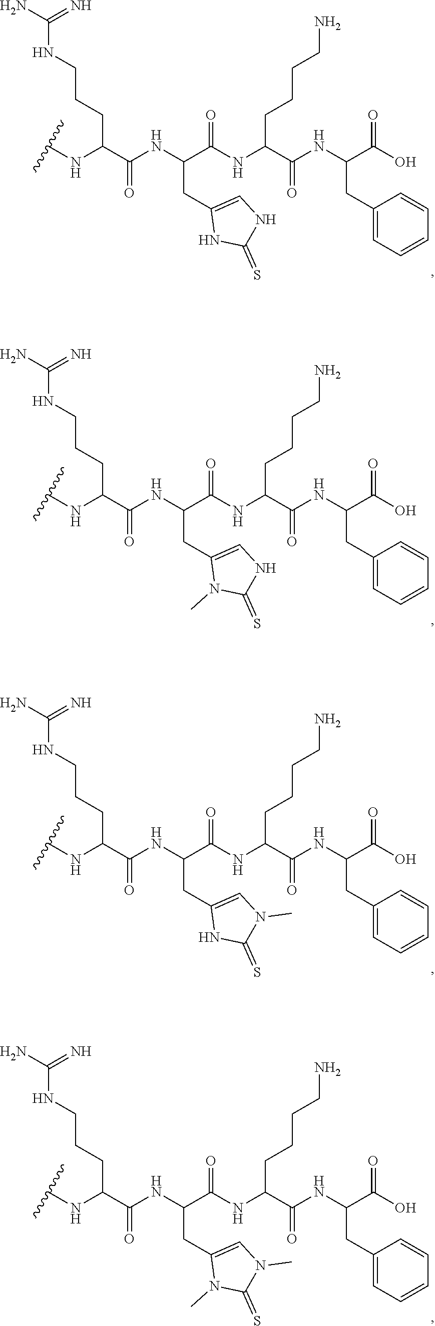 [SMILES: *NC(CCCNC(=N)N)C(=O)NC(Cc1c[nH]c(=S)[nH]1)C(=O)NC(CCCCN)C(=O)NC(Cc1ccccc1)C(=O)O ; *NC(CCCNC(=N)N)C(=O)NC(Cc1c[nH]c(=S)n1C)C(=O)NC(CCCCN)C(=O)NC(Cc1ccccc1)C(=O)O ; *NC(CCCNC(=N)N)C(=O)NC(Cc1cn(C)c(=S)[nH]1)C(=O)NC(CCCCN)C(=O)NC(Cc1ccccc1)C(=O)O ; *NC(CCCNC(=N)N)C(=O)NC(Cc1cn(C)c(=S)n1C)C(=O)NC(CCCCN)C(=O)NC(Cc1ccccc1)C(=O)O]

or protonated forms thereof.

In various embodiments, the amino acid residues of FORMULAS I to IV and any sequence herein may have D or L stereochemistry. In various examples, the stereochemistry of arginine is D and the remainder of the amino acid residues have L stereochemistry. In various examples, when FORMULA I, FORMULA II, FORMULA III, or FORMULA IV is part of a larger peptide or protein, any one of the other amino acid residues have D or L stereochemistry. For example, a peptide or protein comprises or has the following sequence:

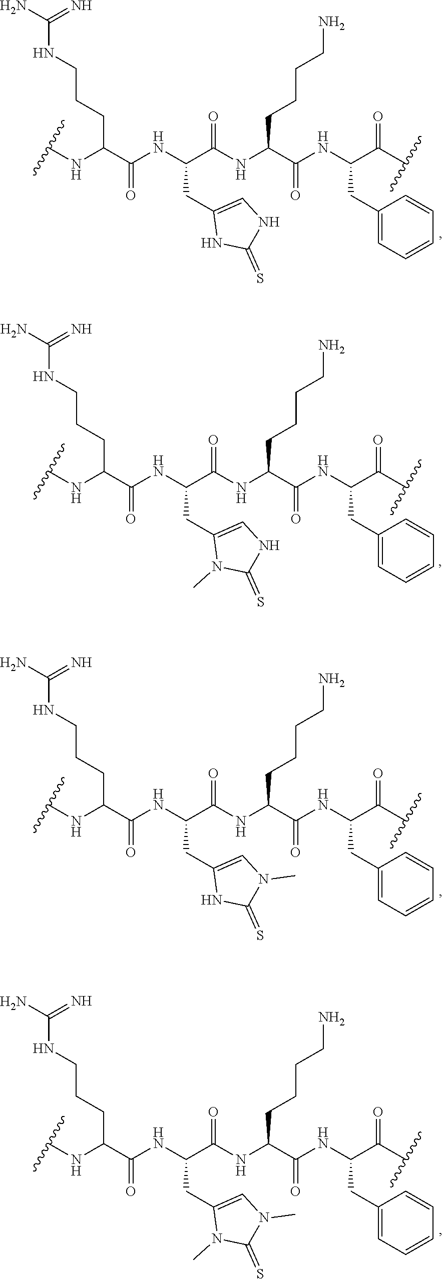 [SMILES: *NC(CCCNC(=N)N)C(=O)N[C@@H](Cc1c[nH]c(=S)[nH]1)C(=O)N[C@@H](CCCCN)C(=O)N[C@@H](Cc1ccccc1)C(*)=O ; *NC(CCCNC(=N)N)C(=O)N[C@@H](Cc1c[nH]c(=S)n1C)C(=O)N[C@@H](CCCCN)C(=O)N[C@@H](Cc1ccccc1)C(*)=O ; *NC(CCCNC(=N)N)C(=O)N[C@@H](Cc1cn(C)c(=S)[nH]1)C(=O)N[C@@H](CCCCN)C(=O)N[C@@H](Cc1ccccc1)C(*)=O ; *NC(CCCNC(=N)N)C(=O)N[C@@H](Cc1cn(C)c(=S)n1C)C(=O)N[C@@H](CCCCN)C(=O)N[C@@H](Cc1ccccc1)C(*)=O]

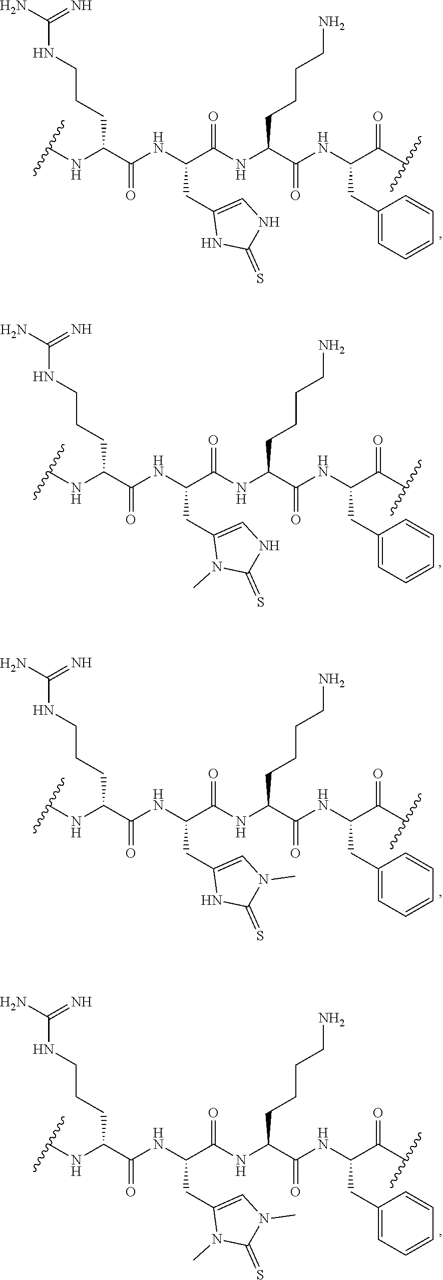 [SMILES: *N[C@H](CCCNC(=N)N)C(=O)N[C@@H](Cc1c[nH]c(=S)[nH]1)C(=O)N[C@@H](CCCCN)C(=O)N[C@@H](Cc1ccccc1)C(*)=O ; *N[C@H](CCCNC(=N)N)C(=O)N[C@@H](Cc1c[nH]c(=S)n1C)C(=O)N[C@@H](CCCCN)C(=O)N[C@@H](Cc1ccccc1)C(*)=O ; *N[C@H](CCCNC(=N)N)C(=O)N[C@@H](Cc1cn(C)c(=S)[nH]1)C(=O)N[C@@H](CCCCN)C(=O)N[C@@H](Cc1ccccc1)C(*)=O ; *N[C@H](CCCNC(=N)N)C(=O)N[C@@H](Cc1cn(C)c(=S)n1C)C(=O)N[C@@H](CCCCN)C(=O)N[C@@H](Cc1ccccc1)C(*)=O]

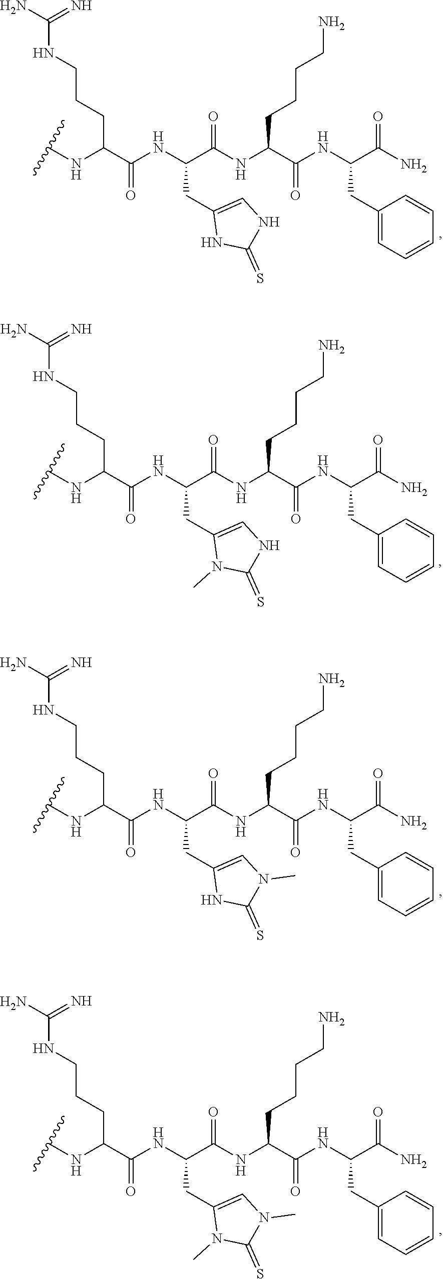 [SMILES: *NC(CCCNC(=N)N)C(=O)N[C@@H](Cc1c[nH]c(=S)[nH]1)C(=O)N[C@@H](CCCCN)C(=O)N[C@@H](Cc1ccccc1)C(N)=O ; *NC(CCCNC(=N)N)C(=O)N[C@@H](Cc1c[nH]c(=S)n1C)C(=O)N[C@@H](CCCCN)C(=O)N[C@@H](Cc1ccccc1)C(N)=O ; *NC(CCCNC(=N)N)C(=O)N[C@@H](Cc1cn(C)c(=S)[nH]1)C(=O)N[C@@H](CCCCN)C(=O)N[C@@H](Cc1ccccc1)C(N)=O ; *NC(CCCNC(=N)N)C(=O)N[C@@H](Cc1cn(C)c(=S)n1C)C(=O)N[C@@H](CCCCN)C(=O)N[C@@H](Cc1ccccc1)C(N)=O]

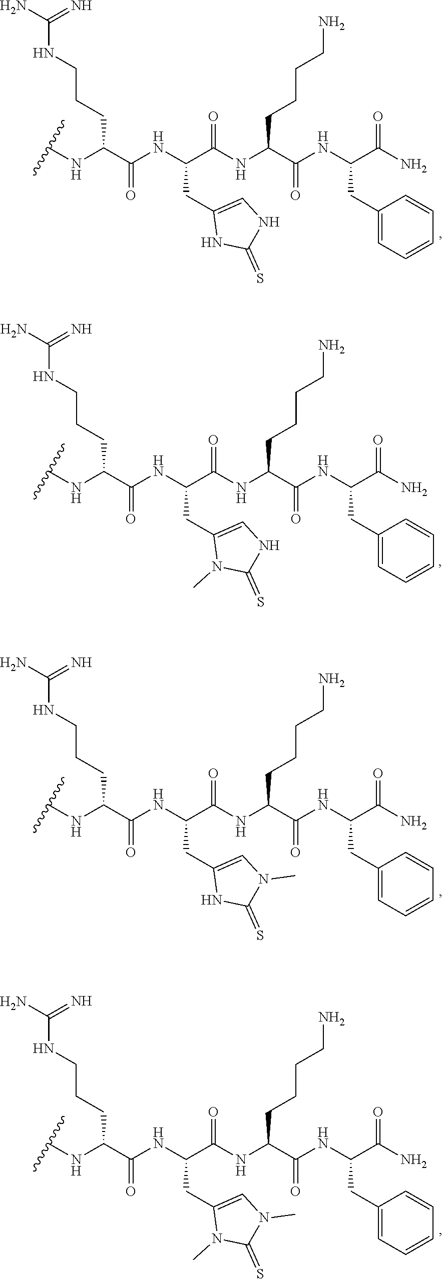 [SMILES: *N[C@H](CCCNC(=N)N)C(=O)N[C@@H](Cc1c[nH]c(=S)[nH]1)C(=O)N[C@@H](CCCCN)C(=O)N[C@@H](Cc1ccccc1)C(N)=O ; *N[C@H](CCCNC(=N)N)C(=O)N[C@@H](Cc1c[nH]c(=S)n1C)C(=O)N[C@@H](CCCCN)C(=O)N[C@@H](Cc1ccccc1)C(N)=O ; *N[C@H](CCCNC(=N)N)C(=O)N[C@@H](Cc1cn(C)c(=S)[nH]1)C(=O)N[C@@H](CCCCN)C(=O)N[C@@H](Cc1ccccc1)C(N)=O ; *N[C@H](CCCNC(=N)N)C(=O)N[C@@H](Cc1cn(C)c(=S)n1C)C(=O)N[C@@H](CCCCN)C(=O)N[C@@H](Cc1ccccc1)C(N)=O]

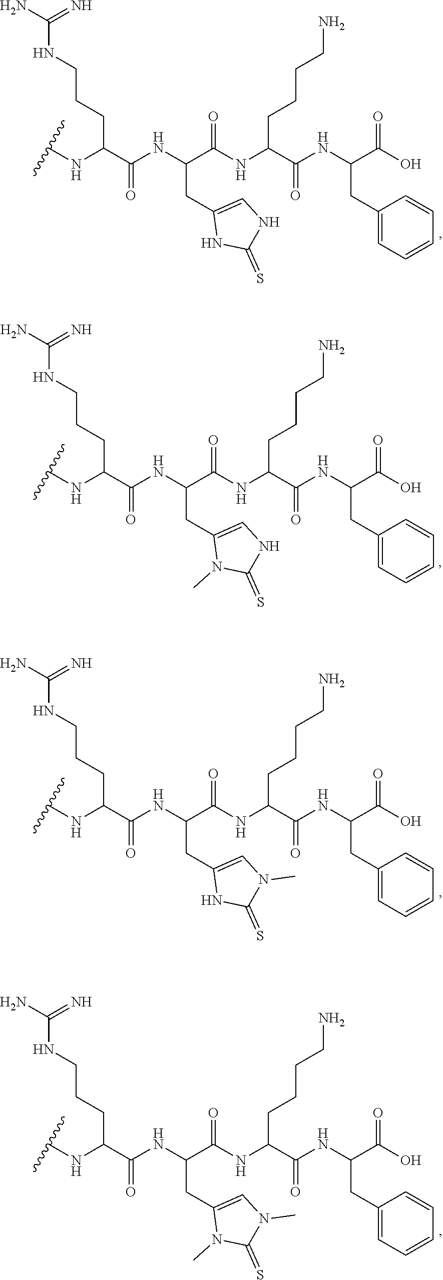 [SMILES: *NC(CCCNC(=N)N)C(=O)NC(Cc1c[nH]c(=S)[nH]1)C(=O)NC(CCCCN)C(=O)NC(Cc1ccccc1)C(=O)O ; *NC(CCCNC(=N)N)C(=O)NC(Cc1c[nH]c(=S)n1C)C(=O)NC(CCCCN)C(=O)NC(Cc1ccccc1)C(=O)O ; *NC(CCCNC(=N)N)C(=O)NC(Cc1cn(C)c(=S)[nH]1)C(=O)NC(CCCCN)C(=O)NC(Cc1ccccc1)C(=O)O ; *NC(CCCNC(=N)N)C(=O)NC(Cc1cn(C)c(=S)n1C)C(=O)NC(CCCCN)C(=O)NC(Cc1ccccc1)C(=O)O]

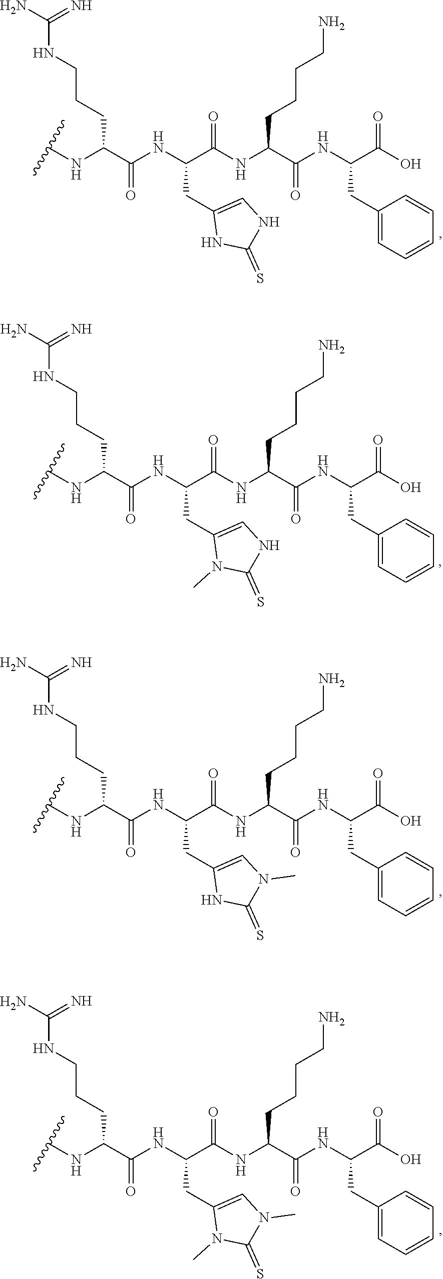 [SMILES: *N[C@H](CCCNC(=N)N)C(=O)N[C@@H](Cc1c[nH]c(=S)[nH]1)C(=O)N[C@@H](CCCCN)C(=O)N[C@@H](Cc1ccccc1)C(=O)O ; *N[C@H](CCCNC(=N)N)C(=O)N[C@@H](Cc1c[nH]c(=S)n1C)C(=O)N[C@@H](CCCCN)C(=O)N[C@@H](Cc1ccccc1)C(=O)O ; *N[C@H](CCCNC(=N)N)C(=O)N[C@@H](Cc1cn(C)c(=S)[nH]1)C(=O)N[C@@H](CCCCN)C(=O)N[C@@H](Cc1ccccc1)C(=O)O ; *N[C@H](CCCNC(=N)N)C(=O)N[C@@H](Cc1cn(C)c(=S)n1C)C(=O)N[C@@H](CCCCN)C(=O)N[C@@H](Cc1ccccc1)C(=O)O]

or protonated forms thereof.

In various examples, the peptide is a tetramer having the following structure:

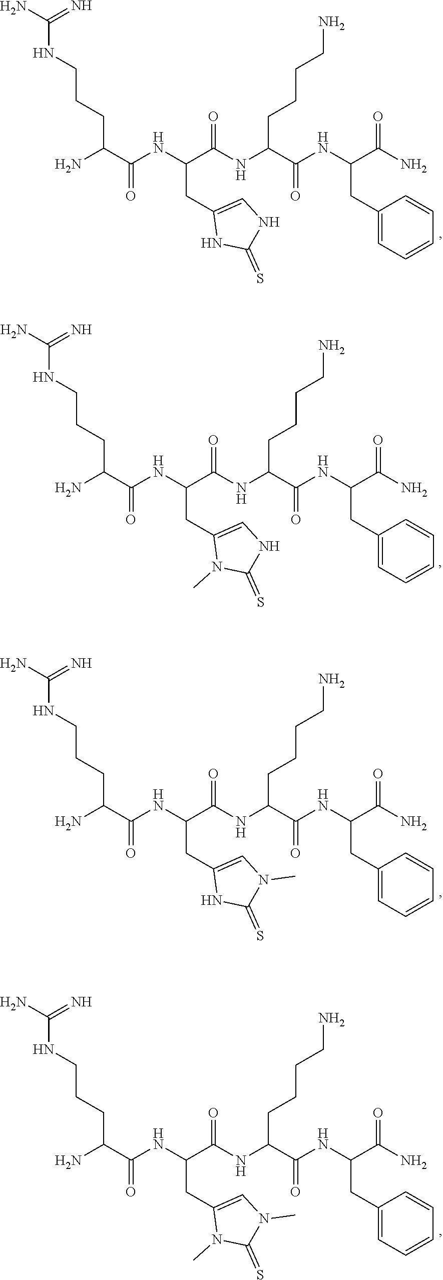 [SMILES: N=C(N)NCCCC(N)C(=O)NC(Cc1c[nH]c(=S)[nH]1)C(=O)NC(CCCCN)C(=O)NC(Cc1ccccc1)C(N)=O ; Cn1c(CC(NC(=O)C(N)CCCNC(=N)N)C(=O)NC(CCCCN)C(=O)NC(Cc2ccccc2)C(N)=O)c[nH]c1=S ; Cn1cc(CC(NC(=O)C(N)CCCNC(=N)N)C(=O)NC(CCCCN)C(=O)NC(Cc2ccccc2)C(N)=O)[nH]c1=S ; Cn1cc(CC(NC(=O)C(N)CCCNC(=N)N)C(=O)NC(CCCCN)C(=O)NC(Cc2ccccc2)C(N)=O)n(C)c1=S]

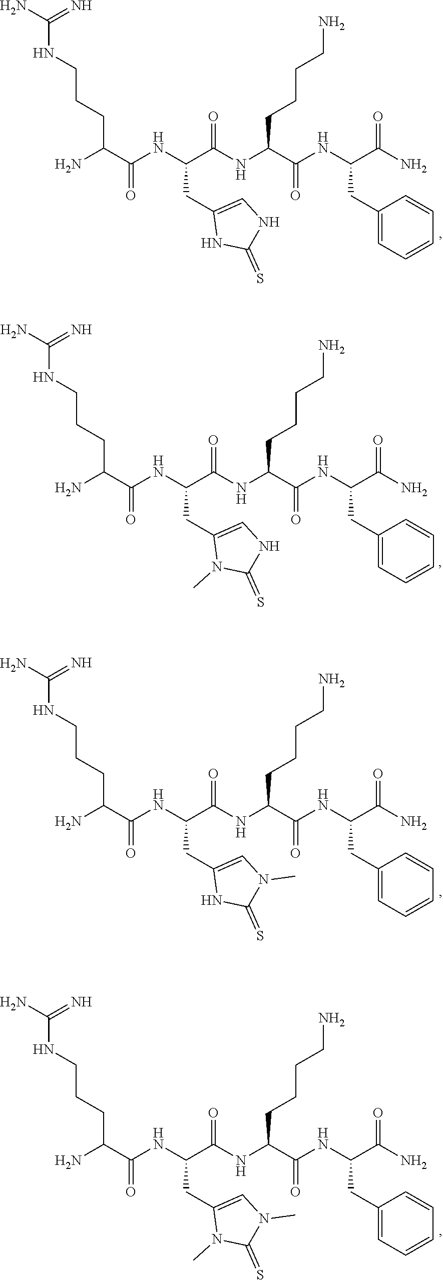 [SMILES: N=C(N)NCCCC(N)C(=O)N[C@@H](Cc1c[nH]c(=S)[nH]1)C(=O)N[C@@H](CCCCN)C(=O)N[C@@H](Cc1ccccc1)C(N)=O ; Cn1c(C[C@H](NC(=O)C(N)CCCNC(=N)N)C(=O)N[C@@H](CCCCN)C(=O)N[C@@H](Cc2ccccc2)C(N)=O)c[nH]c1=S ; Cn1cc(C[C@H](NC(=O)C(N)CCCNC(=N)N)C(=O)N[C@@H](CCCCN)C(=O)N[C@@H](Cc2ccccc2)C(N)=O)[nH]c1=S ; Cn1cc(C[C@H](NC(=O)C(N)CCCNC(=N)N)C(=O)N[C@@H](CCCCN)C(=O)N[C@@H](Cc2ccccc2)C(N)=O)n(C)c1=S]

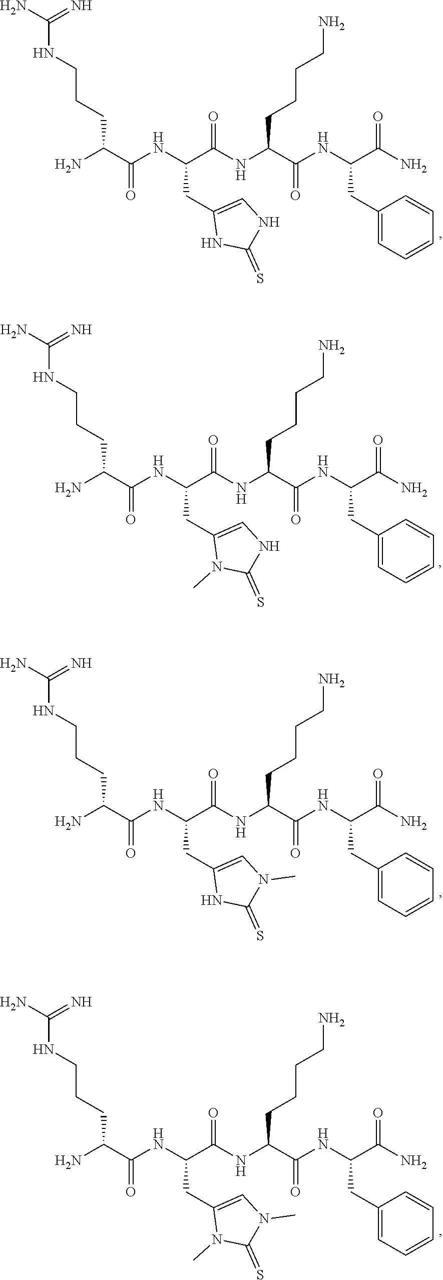 [SMILES: N=C(N)NCCC[C@@H](N)C(=O)N[C@@H](Cc1c[nH]c(=S)[nH]1)C(=O)N[C@@H](CCCCN)C(=O)N[C@@H](Cc1ccccc1)C(N)=O ; Cn1c(C[C@H](NC(=O)[C@H](N)CCCNC(=N)N)C(=O)N[C@@H](CCCCN)C(=O)N[C@@H](Cc2ccccc2)C(N)=O)c[nH]c1=S ; Cn1cc(C[C@H](NC(=O)[C@H](N)CCCNC(=N)N)C(=O)N[C@@H](CCCCN)C(=O)N[C@@H](Cc2ccccc2)C(N)=O)[nH]c1=S ; Cn1cc(C[C@H](NC(=O)[C@H](N)CCCNC(=N)N)C(=O)N[C@@H](CCCCN)C(=O)N[C@@H](Cc2ccccc2)C(N)=O)n(C)c1=S]

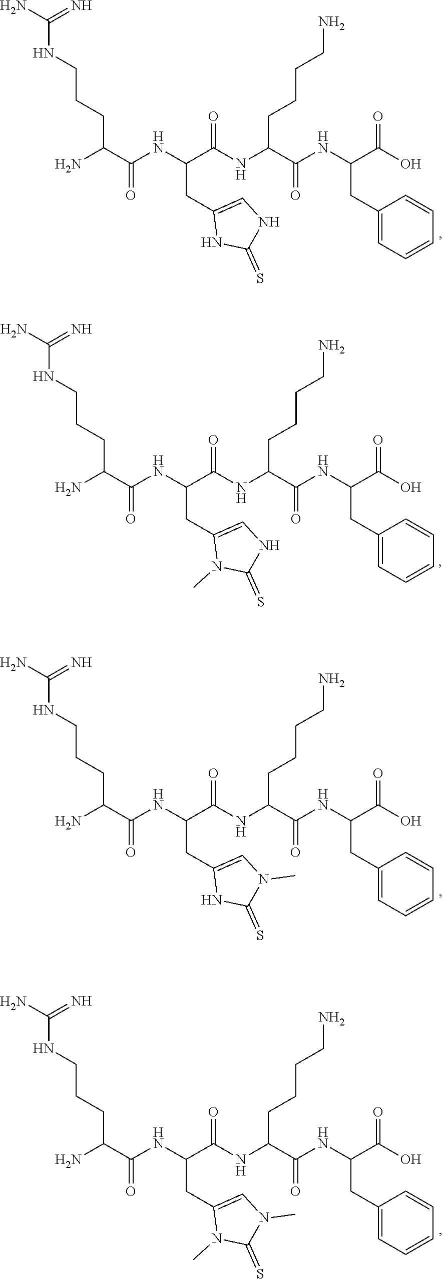 [SMILES: N=C(N)NCCCC(N)C(=O)NC(Cc1c[nH]c(=S)[nH]1)C(=O)NC(CCCCN)C(=O)NC(Cc1ccccc1)C(=O)O ; Cn1c(CC(NC(=O)C(N)CCCNC(=N)N)C(=O)NC(CCCCN)C(=O)NC(Cc2ccccc2)C(=O)O)c[nH]c1=S ; Cn1cc(CC(NC(=O)C(N)CCCNC(=N)N)C(=O)NC(CCCCN)C(=O)NC(Cc2ccccc2)C(=O)O)[nH]c1=S ; Cn1cc(CC(NC(=O)C(N)CCCNC(=N)N)C(=O)NC(CCCCN)C(=O)NC(Cc2ccccc2)C(=O)O)n(C)c1=S]

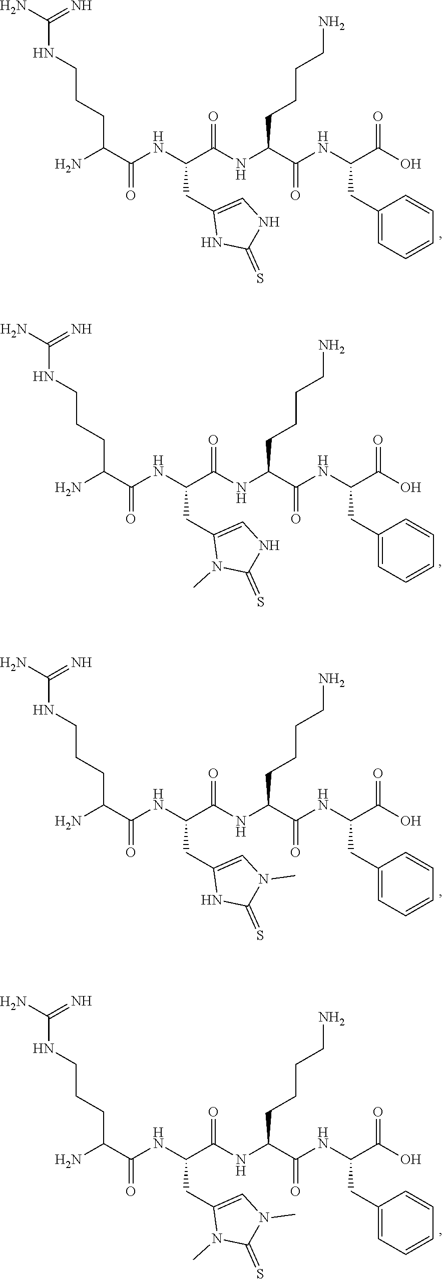 [SMILES: N=C(N)NCCCC(N)C(=O)N[C@@H](Cc1c[nH]c(=S)[nH]1)C(=O)N[C@@H](CCCCN)C(=O)N[C@@H](Cc1ccccc1)C(=O)O ; Cn1c(C[C@H](NC(=O)C(N)CCCNC(=N)N)C(=O)N[C@@H](CCCCN)C(=O)N[C@@H](Cc2ccccc2)C(=O)O)c[nH]c1=S ; Cn1cc(C[C@H](NC(=O)C(N)CCCNC(=N)N)C(=O)N[C@@H](CCCCN)C(=O)N[C@@H](Cc2ccccc2)C(=O)O)[nH]c1=S ; Cn1cc(C[C@H](NC(=O)C(N)CCCNC(=N)N)C(=O)N[C@@H](CCCCN)C(=O)N[C@@H](Cc2ccccc2)C(=O)O)n(C)c1=S]

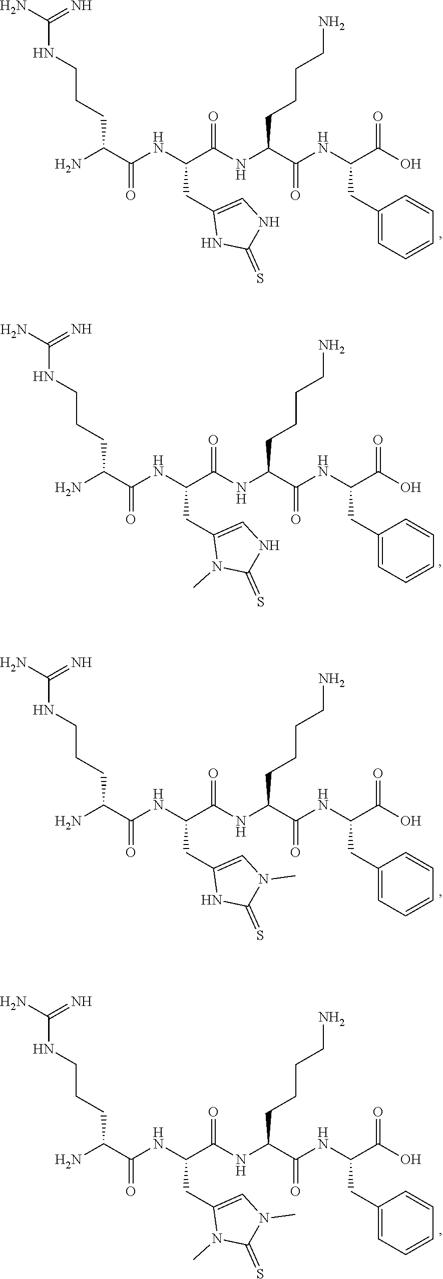 [SMILES: N=C(N)NCCC[C@@H](N)C(=O)N[C@@H](Cc1c[nH]c(=S)[nH]1)C(=O)N[C@@H](CCCCN)C(=O)N[C@@H](Cc1ccccc1)C(=O)O ; Cn1c(C[C@H](NC(=O)[C@H](N)CCCNC(=N)N)C(=O)N[C@@H](CCCCN)C(=O)N[C@@H](Cc2ccccc2)C(=O)O)c[nH]c1=S ; Cn1cc(C[C@H](NC(=O)[C@H](N)CCCNC(=N)N)C(=O)N[C@@H](CCCCN)C(=O)N[C@@H](Cc2ccccc2)C(=O)O)[nH]c1=S ; Cn1cc(C[C@H](NC(=O)[C@H](N)CCCNC(=N)N)C(=O)N[C@@H](CCCCN)C(=O)N[C@@H](Cc2ccccc2)C(=O)O)n(C)c1=S]

or protonated forms thereof.

In various examples, the N-terminus may be functionalized. For example, the N-terminus is functionalized with a capping group, a linking group, or a linking group covalently attached to a capping group. Examples of capping groups include, but are not limited to, acetyl groups, fatty acid groups (e.g., palmitoyl groups), vitamins (e.g., ascorbic acid), saccharides (e.g., mono or polysaccharides), and the like. Examples of linking groups include, but are not limited to succinyl groups, polyethylene glycol groups, saccharides (e.g., mono or polysaccharides), and the like, and combinations thereof.

In an aspect, the present disclosure provides compositions. The composition may comprise a peptide of the present disclosure and a pharmaceutically acceptable carrier.

The composition can comprise the peptides in a pharmaceutically acceptable carrier (e.g., carrier). The carrier can be an aqueous carrier suitable for administration to individuals including humans. The carrier can be sterile. The carrier can be a physiological buffer. Examples of suitable carriers include sucrose, dextrose, saline, and/or a pH buffering element (such as, a buffering element that buffers to, for example, a pH from pH 5 to 9, from pH 6 to 8, (e.g., 6.5)) such as histidine, citrate, or phosphate. Additionally, pharmaceutically acceptable carriers may be determined in part by the particular composition being administered. Accordingly, there are a wide variety of suitable formulations of pharmaceutical compositions of the present disclosure. Additional, non-limiting examples of carriers include solutions, suspensions, and emulsions that are dissolved or suspended in a solvent before use, and the like. The composition may comprise one or more diluents. Examples of diluents, include, but are not limited to distilled water, physiological saline, vegetable oil, alcohol, dimethyl sulfoxide, and the like, and combinations thereof. Compositions may contain stabilizers, solubilizers, suspending agents, emulsifiers, soothing agents, buffers, preservatives, and the like, and combinations thereof. Compositions may be sterilized or prepared by sterile procedure. A composition of the disclosure may also be formulated into a sterile solid preparation, for example, by freeze-drying, and may be used after sterilization or dissolution in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2012) 22nd Edition, Philadelphia, PA. Lippincott Williams & Wilkins. For example, a composition comprises a modified peptide, and a sterile, suitable carrier for administration to individuals including humans—such as a physiological buffer such as sucrose, dextrose, saline, pH buffering (such as from pH 5 to 9, from pH 7 to 8, from pH 7.2 to 7.6, (e.g., 7.4)) element such as, for example, histidine, citrate, or phosphate. In various examples, the composition may be suitable for injection. Parenteral administration includes infusions and injections, such as, for example, intramuscular, intravenous, intraarterial, intraperitoneal, subcutaneous administration, and the like.

The compositions may be administered systemically. Compositions may be administered orally, may be administered parenterally, and/or intravenously. Compositions suitable for parenteral, administration may include aqueous and/or non-aqueous carriers and diluents, such as, for example, sterile injection solutions. Sterile injection solutions may contain anti-oxidants, buffers, bacteriostatic agents and solutes, which render the composition isotonic with the blood of the intended recipient. Aqueous and/or non-aqueous sterile suspensions may include suspending agents and thickening agents.

Nasal aerosol and inhalation compositions of the present disclosure may be prepared by any method in the art. Such compositions may include dosing vehicles, such as, for example, saline; preservatives, such as, for example, benzyl alcohol; absorption promoters to enhance bioavailability; fluorocarbons used in the delivery systems (e.g., nebulizers and the like; solubilizing agents; dispersing agents; or a combination thereof).

The compositions of the present disclosure may be administered systemically. The term “systemic” as used herein includes parenteral, topical, oral, spray inhalation, rectal, nasal, and buccal administration. The term “parenteral” as used herein includes subcutaneous, intravenous, intramuscular, intra-articular, intra-synovial, intrasternal, intrathecal, intrahepatic, intralesional, and intracranial administration. Preferably, the compositions are administered orally, intraperitoneally, or intravenously.

Examples of compositions include, but are not limited to, liquid solutions, such as, for example, an effective amount of a compound of the present disclosure suspended in diluents, such as, for example, water, saline or PEG 400. The liquid solutions described above may be sterile solutions. The compositions may comprise, for example, one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers.

Peptides of the present disclosure may be prepared by utilizing SPPS, where

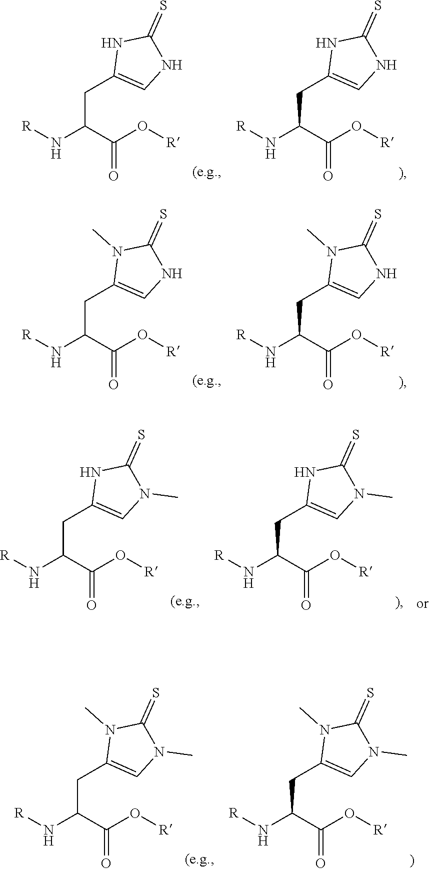

is reacted with a nucleophilic group of a peptide or amino acid residues covalently attached to a resin. R is an amine protecting group, such as, for example, Fmoc and R′ is H or a group formed from a carbodiimide (e.g., the —OR′ is an activated ester). Aside from the N-terminal protecting group, 2-thioHis or its methylated variants have no other protecting groups. Specifically, the thione of 2-thioHis (or its methylated variants) is not protected during reaction (e.g., coupling) of 2-thioHis (or its methylated variants). The nucleophilic group may be the N-terminal amine of a peptide covalently attached to a resin. Following attachment (e.g., coupling) of 2-thioHis (or its methylated variants) to the peptide or amino acid residues covalently attached to a resin, the amine protecting group of the 2-thioHis (or its methylated variants) may be removed from the amine group of 2-thioHis (or its methylated variants) and additional amino acid(s) may be sequentially added to the peptide chain using standard SPPS techniques known in the art.

Various amino acids may be used in a method of the present disclosure. Examples of amino acids include, but are not limited to, canonical amino acids, non-canonical amino acids, amino acid derivatives, and the like, and combinations thereof.

Various resins may be used. For example, non-limiting examples of resins are Rink amide resins, PAL resins, Sieber Amide resins, Wang resins, trityl resins, chlorotrityl resins, and the like. Other resins are known in the art and are considered within the scope of the present disclosure.

Various activators may be used when synthesizing a peptide of the present disclosure. For example, activator may be a carbodiimide. For example, suitable carbodiimides include, but are not limited to, diisopropylcarbodiimide (DIC), 1-hydroxybenzotriazole (HOBt), 1-hydroxy-7-azabenzotriazole (HOAt), 2-(1H-benzotriazol-1-yl)-1,1,3,3-tetramethyluronium hexafluorophosphate (HBTU), 1-[Bis(dimethylamino)methylene]-1H-1,2,3-triazolo[4,5-b]pyridinium 3-oxide hexafluorophosphate (HATU), and the like, and combinations thereof. The carbodiimides may be used to form the carbodiimide group of R′.

The method may further comprise cleaving the peptide from the solid support (e.g., resin). Methods of cleaving a peptide from resins are known in the art. For example, cleavage comprises contacting the modified peptide covalently attached to resin with a cleavage cocktail. For Fmoc-based chemistries, the cleavage cocktail may comprise trifluoroacetic acid (TFA). The cleavage cocktail may further comprise a silane (e.g., triisopropylsilane (TIS)) and water. Other cleavage cocktails include, but are not limited to, Reagent K, Reagent L, and Reagent R. Following cleavage, the solution comprising the cleaved modified peptide may be concentrated. Then, the modified peptide may be precipitated from the concentrated solution using, for example, cold, anhydrous ether. The precipitated peptide may be pelleted and then purified.

Methods to purify the peptides include using peptide purification methods known in the art. For example, purification may comprise utilizing high performance liquid chromatography (e.g., HPLC) or other similar methods. HPLC may be reverse-phase HPLC, where acetonitrile (with or without TFA) and water (with or without TFA) is the mobile phase. Various other mobile phases are known in the art and are within the scope of the present disclosure. The aqueous fractions containing the purified modified peptides may be concentrated using methods known in the art. For example, the aqueous fractions are lyophilized.

In an aspect, the present disclosure provides methods of using peptides of the present disclosure. The method may be a method for treating an individual having or suspected of having a disease associated with mitochondrial dysfunction and/or diseases associated with HOCl-mediated injury, comprising administering to the individual a composition of the present disclosure.

In various examples, the composition comprises a peptide having the following structure:

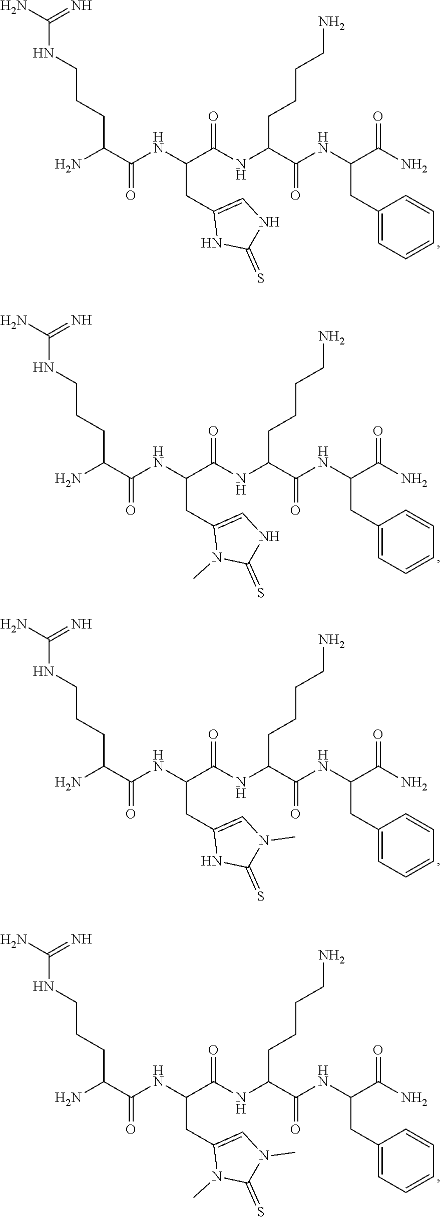 [SMILES: N=C(N)NCCCC(N)C(=O)NC(Cc1c[nH]c(=S)[nH]1)C(=O)NC(CCCCN)C(=O)NC(Cc1ccccc1)C(N)=O ; Cn1c(CC(NC(=O)C(N)CCCNC(=N)N)C(=O)NC(CCCCN)C(=O)NC(Cc2ccccc2)C(N)=O)c[nH]c1=S ; Cn1cc(CC(NC(=O)C(N)CCCNC(=N)N)C(=O)NC(CCCCN)C(=O)NC(Cc2ccccc2)C(N)=O)[nH]c1=S ; Cn1cc(CC(NC(=O)C(N)CCCNC(=N)N)C(=O)NC(CCCCN)C(=O)NC(Cc2ccccc2)C(N)=O)n(C)c1=S]

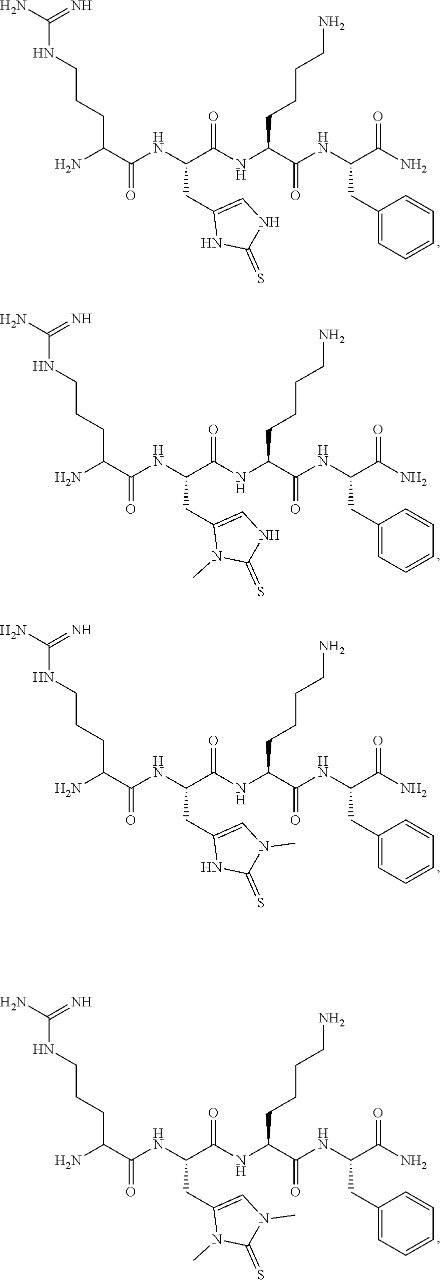 [SMILES: N=C(N)NCCCC(N)C(=O)N[C@@H](Cc1c[nH]c(=S)[nH]1)C(=O)N[C@@H](CCCCN)C(=O)N[C@@H](Cc1ccccc1)C(N)=O ; Cn1c(C[C@H](NC(=O)C(N)CCCNC(=N)N)C(=O)N[C@@H](CCCCN)C(=O)N[C@@H](Cc2ccccc2)C(N)=O)c[nH]c1=S ; Cn1cc(C[C@H](NC(=O)C(N)CCCNC(=N)N)C(=O)N[C@@H](CCCCN)C(=O)N[C@@H](Cc2ccccc2)C(N)=O)[nH]c1=S ; Cn1cc(C[C@H](NC(=O)C(N)CCCNC(=N)N)C(=O)N[C@@H](CCCCN)C(=O)N[C@@H](Cc2ccccc2)C(N)=O)n(C)c1=S]

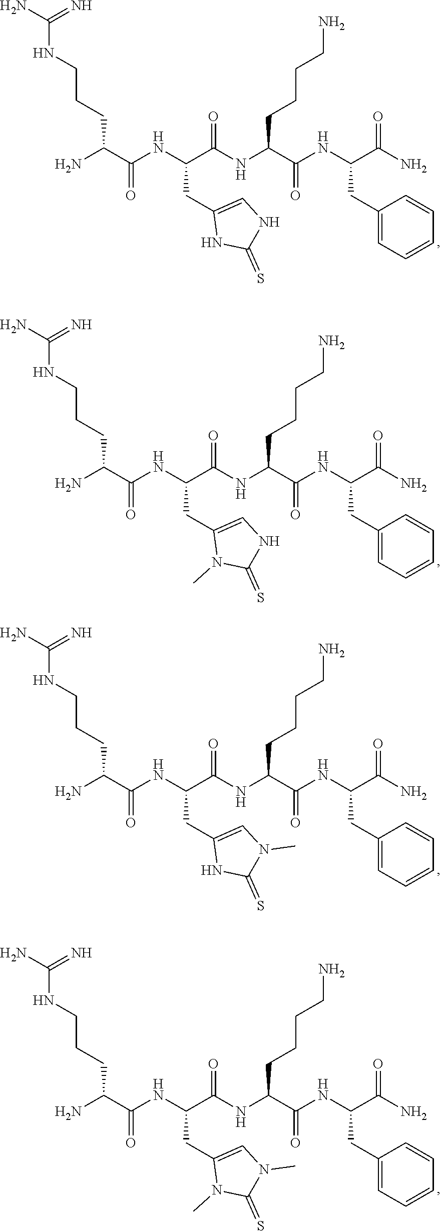 [SMILES: N=C(N)NCCC[C@@H](N)C(=O)N[C@@H](Cc1c[nH]c(=S)[nH]1)C(=O)N[C@@H](CCCCN)C(=O)N[C@@H](Cc1ccccc1)C(N)=O ; Cn1c(C[C@H](NC(=O)[C@H](N)CCCNC(=N)N)C(=O)N[C@@H](CCCCN)C(=O)N[C@@H](Cc2ccccc2)C(N)=O)c[nH]c1=S ; Cn1cc(C[C@H](NC(=O)[C@H](N)CCCNC(=N)N)C(=O)N[C@@H](CCCCN)C(=O)N[C@@H](Cc2ccccc2)C(N)=O)[nH]c1=S ; Cn1cc(C[C@H](NC(=O)[C@H](N)CCCNC(=N)N)C(=O)N[C@@H](CCCCN)C(=O)N[C@@H](Cc2ccccc2)C(N)=O)n(C)c1=S]

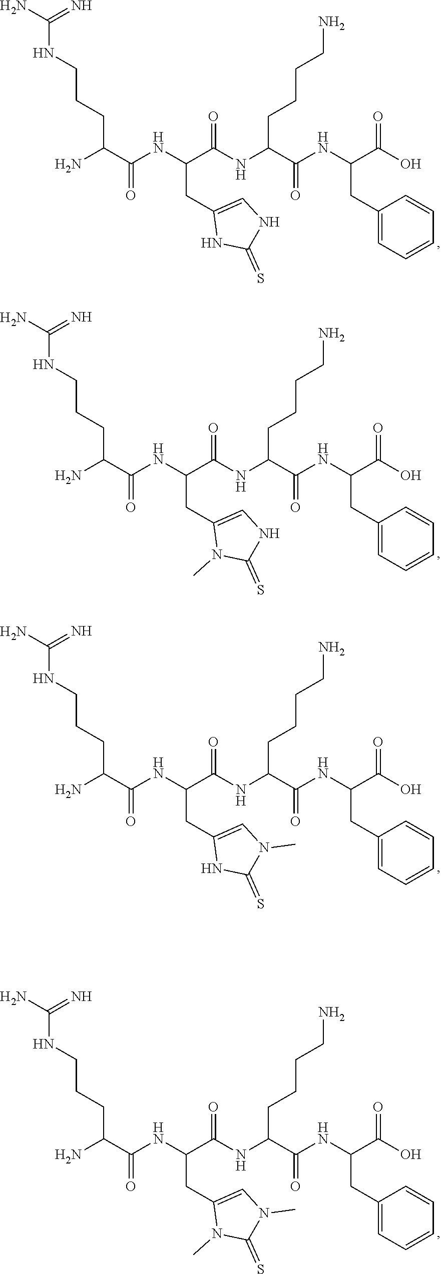 [SMILES: N=C(N)NCCCC(N)C(=O)NC(Cc1c[nH]c(=S)[nH]1)C(=O)NC(CCCCN)C(=O)NC(Cc1ccccc1)C(=O)O ; Cn1c(CC(NC(=O)C(N)CCCNC(=N)N)C(=O)NC(CCCCN)C(=O)NC(Cc2ccccc2)C(=O)O)c[nH]c1=S ; Cn1cc(CC(NC(=O)C(N)CCCNC(=N)N)C(=O)NC(CCCCN)C(=O)NC(Cc2ccccc2)C(=O)O)[nH]c1=S ; Cn1cc(CC(NC(=O)C(N)CCCNC(=N)N)C(=O)NC(CCCCN)C(=O)NC(Cc2ccccc2)C(=O)O)n(C)c1=S]

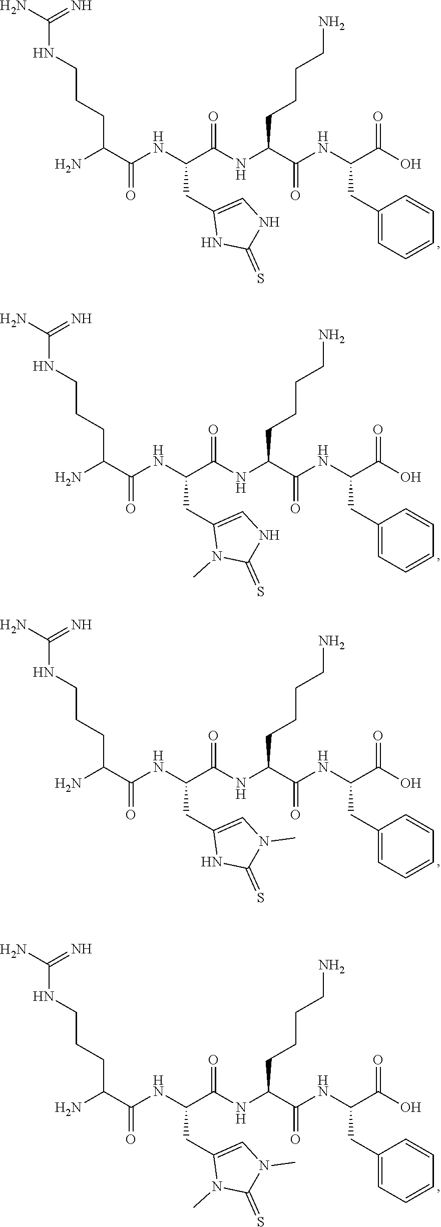 [SMILES: N=C(N)NCCCC(N)C(=O)N[C@@H](Cc1c[nH]c(=S)[nH]1)C(=O)N[C@@H](CCCCN)C(=O)N[C@@H](Cc1ccccc1)C(=O)O ; Cn1c(C[C@H](NC(=O)C(N)CCCNC(=N)N)C(=O)N[C@@H](CCCCN)C(=O)N[C@@H](Cc2ccccc2)C(=O)O)c[nH]c1=S ; Cn1cc(C[C@H](NC(=O)C(N)CCCNC(=N)N)C(=O)N[C@@H](CCCCN)C(=O)N[C@@H](Cc2ccccc2)C(=O)O)[nH]c1=S ; Cn1cc(C[C@H](NC(=O)C(N)CCCNC(=N)N)C(=O)N[C@@H](CCCCN)C(=O)N[C@@H](Cc2ccccc2)C(=O)O)n(C)c1=S]

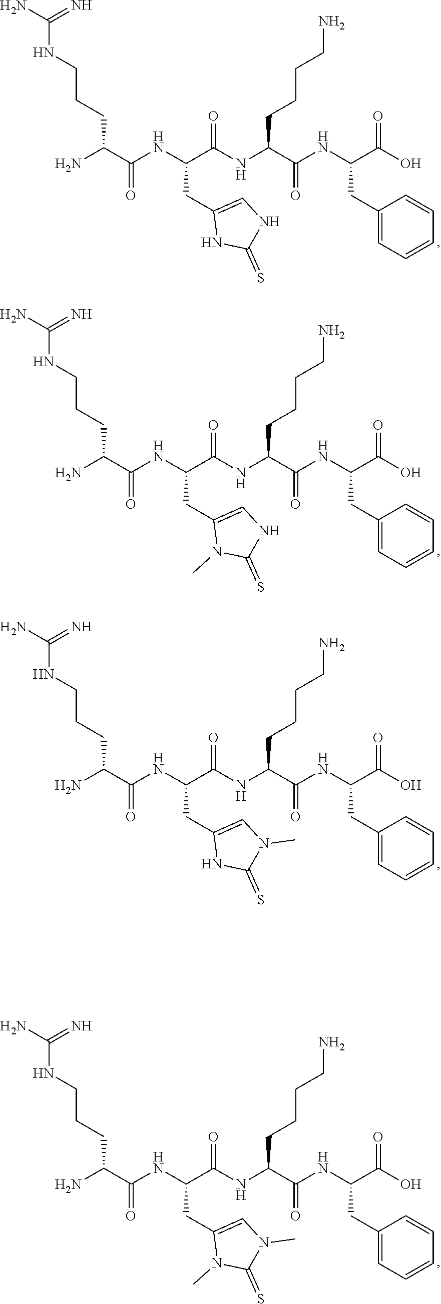 [SMILES: N=C(N)NCCC[C@@H](N)C(=O)N[C@@H](Cc1c[nH]c(=S)[nH]1)C(=O)N[C@@H](CCCCN)C(=O)N[C@@H](Cc1ccccc1)C(=O)O ; Cn1c(C[C@H](NC(=O)[C@H](N)CCCNC(=N)N)C(=O)N[C@@H](CCCCN)C(=O)N[C@@H](Cc2ccccc2)C(=O)O)c[nH]c1=S ; Cn1cc(C[C@H](NC(=O)[C@H](N)CCCNC(=N)N)C(=O)N[C@@H](CCCCN)C(=O)N[C@@H](Cc2ccccc2)C(=O)O)[nH]c1=S ; Cn1cc(C[C@H](NC(=O)[C@H](N)CCCNC(=N)N)C(=O)N[C@@H](CCCCN)C(=O)N[C@@H](Cc2ccccc2)C(=O)O)n(C)c1=S]

or protonated forms thereof. In various examples, the peptide is:

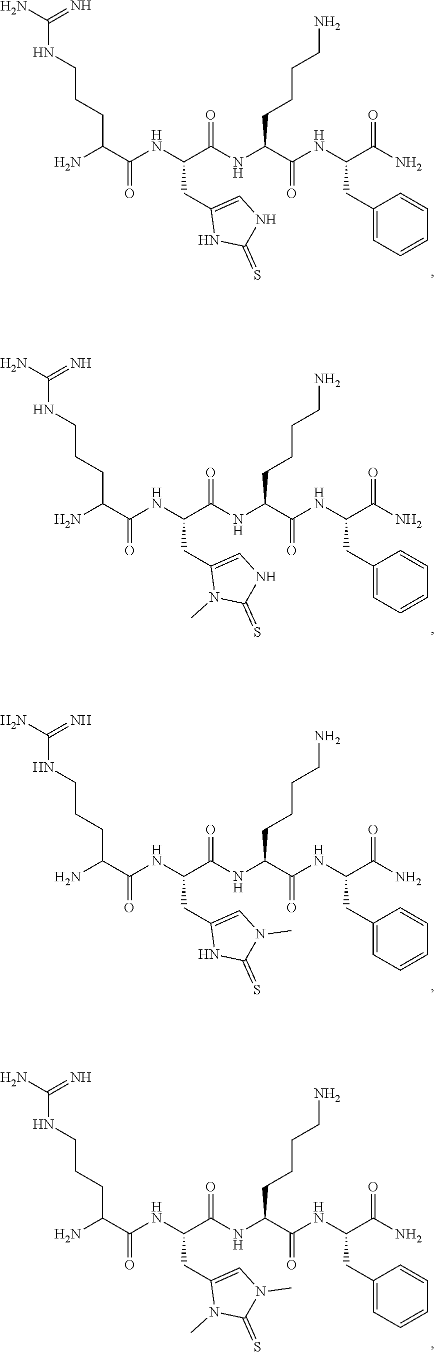 [SMILES: N=C(N)NCCCC(N)C(=O)N[C@@H](Cc1c[nH]c(=S)[nH]1)C(=O)N[C@@H](CCCCN)C(=O)N[C@@H](Cc1ccccc1)C(N)=O ; Cn1c(C[C@H](NC(=O)C(N)CCCNC(=N)N)C(=O)N[C@@H](CCCCN)C(=O)N[C@@H](Cc2ccccc2)C(N)=O)c[nH]c1=S ; Cn1cc(C[C@H](NC(=O)C(N)CCCNC(=N)N)C(=O)N[C@@H](CCCCN)C(=O)N[C@@H](Cc2ccccc2)C(N)=O)[nH]c1=S ; Cn1cc(C[C@H](NC(=O)C(N)CCCNC(=N)N)C(=O)N[C@@H](CCCCN)C(=O)N[C@@H](Cc2ccccc2)C(N)=O)n(C)c1=S]

or a protonated analogue thereof.

In various examples, a peptide or protein of the present disclosure may be used to protect against chlorine gas exposure. For example, a composition comprising a peptide of the present disclosure is dosed during exposure to Cl₂.

The phrase “therapeutically effective amount” is used herein to mean an amount sufficient to reduce by at least about 15 percent, preferably by at least 50 percent, more preferably by at least 90 percent, and most preferably prevents oxidative stress in the individual. Alternatively, a therapeutically effective amount is sufficient to cause an improvement in a clinically significant condition in the host.

In various examples, a disease that may be ameliorated by a method of the present disclosure is Duchenne's muscular dystrophy, age-related macular degeneration, Barth syndrome, Leber's hereditary optic neuropathy, or a combination thereof.

In a method of the present disclosure, compositions may be administered by various routes. The compositions of the present disclosure may be administered systemically or orally.

An individual in need of treatment may be a human or non-human mammal. Non-limiting examples of non-human mammals include cows, pigs, mice, rats, rabbits, cats, dogs, other agricultural animal, pet, service animals, and the like.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

In an aspect, the present disclosure also provides kits comprising the peptides or proteins of the present disclosure. The kits may also comprise instructions for use.

The peptide or protein may be present in various forms. For example, the peptide or protein may be in solution, where the solvent is a diluent or pharmaceutically acceptable carrier. In other embodiments, the peptide or protein is a lyophilized powder. The powder may be a salt (e.g., a TFA salt).

The following Statements provide various examples of the present disclosure.
Statement 1. A peptide or protein comprising the following sequence: -RXKF-, where X is 2TH residue or a 2TH residue analogue

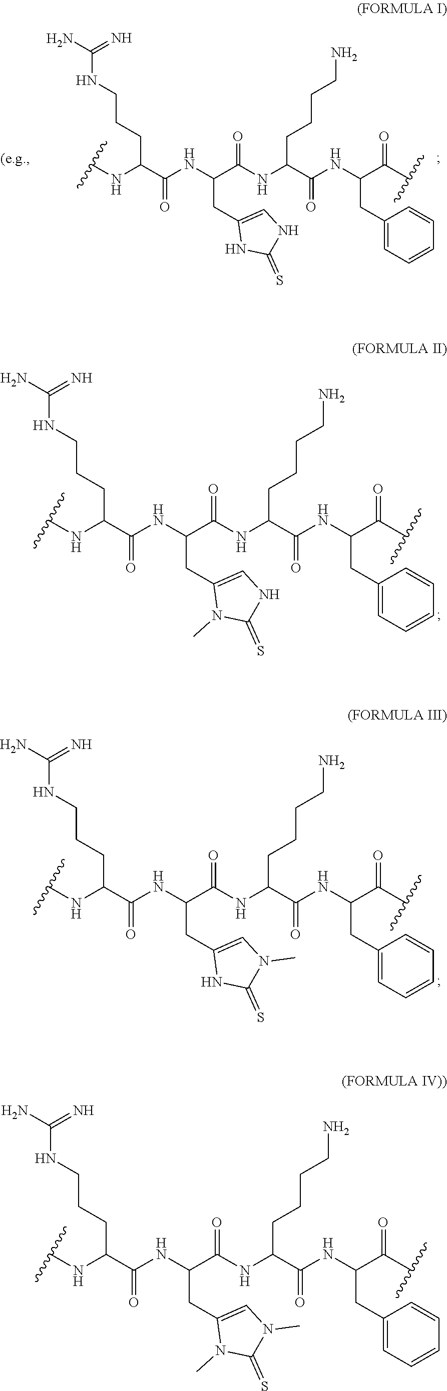 [SMILES: *NC(CCCNC(=N)N)C(=O)NC(Cc1c[nH]c(=S)[nH]1)C(=O)NC(CCCCN)C(=O)NC(Cc1ccccc1)C(*)=O ; *NC(CCCNC(=N)N)C(=O)NC(Cc1c[nH]c(=S)n1C)C(=O)NC(CCCCN)C(=O)NC(Cc1ccccc1)C(*)=O ; *NC(CCCNC(=N)N)C(=O)NC(Cc1cn(C)c(=S)[nH]1)C(=O)NC(CCCCN)C(=O)NC(Cc1ccccc1)C(*)=O ; *NC(CCCNC(=N)N)C(=O)NC(Cc1cn(C)c(=S)n1C)C(=O)NC(CCCCN)C(=O)NC(Cc1ccccc1)C(*)=O]

or a protonated analogue thereof.
Statement 2. A peptide or protein according to Statement 1, wherein the peptide or protein comprises the following sequence:

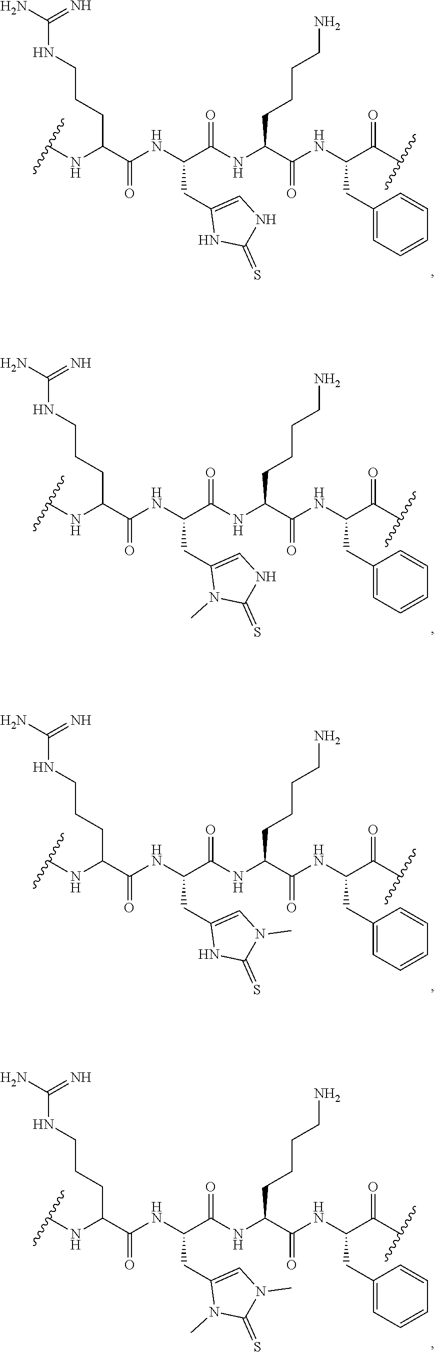 [SMILES: *NC(CCCNC(=N)N)C(=O)N[C@@H](Cc1c[nH]c(=S)[nH]1)C(=O)N[C@@H](CCCCN)C(=O)N[C@@H](Cc1ccccc1)C(*)=O ; *NC(CCCNC(=N)N)C(=O)N[C@@H](Cc1c[nH]c(=S)n1C)C(=O)N[C@@H](CCCCN)C(=O)N[C@@H](Cc1ccccc1)C(*)=O ; *NC(CCCNC(=N)N)C(=O)N[C@@H](Cc1cn(C)c(=S)[nH]1)C(=O)N[C@@H](CCCCN)C(=O)N[C@@H](Cc1ccccc1)C(*)=O ; *NC(CCCNC(=N)N)C(=O)N[C@@H](Cc1cn(C)c(=S)n1C)C(=O)N[C@@H](CCCCN)C(=O)N[C@@H](Cc1ccccc1)C(*)=O]

or a protonated analogue thereof.
Statement 3. A peptide or protein according to Statement 1, wherein the peptide or protein comprises the following sequence:

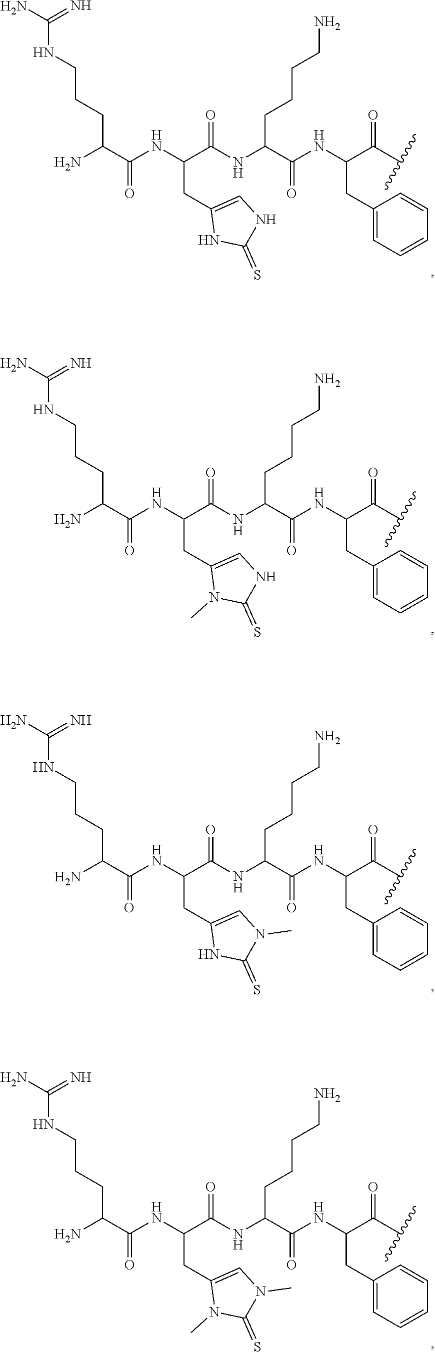 [SMILES: *C(=O)C(Cc1ccccc1)NC(=O)C(CCCCN)NC(=O)C(Cc1c[nH]c(=S)[nH]1)NC(=O)C(N)CCCNC(=N)N ; *C(=O)C(Cc1ccccc1)NC(=O)C(CCCCN)NC(=O)C(Cc1c[nH]c(=S)n1C)NC(=O)C(N)CCCNC(=N)N ; *C(=O)C(Cc1ccccc1)NC(=O)C(CCCCN)NC(=O)C(Cc1cn(C)c(=S)[nH]1)NC(=O)C(N)CCCNC(=N)N ; *C(=O)C(Cc1ccccc1)NC(=O)C(CCCCN)NC(=O)C(Cc1cn(C)c(=S)n1C)NC(=O)C(N)CCCNC(=N)N]

or a protonated analogue thereof.
Statement 4. A peptide or protein according to the Statement 3, wherein the peptide or protein comprises the following sequence:

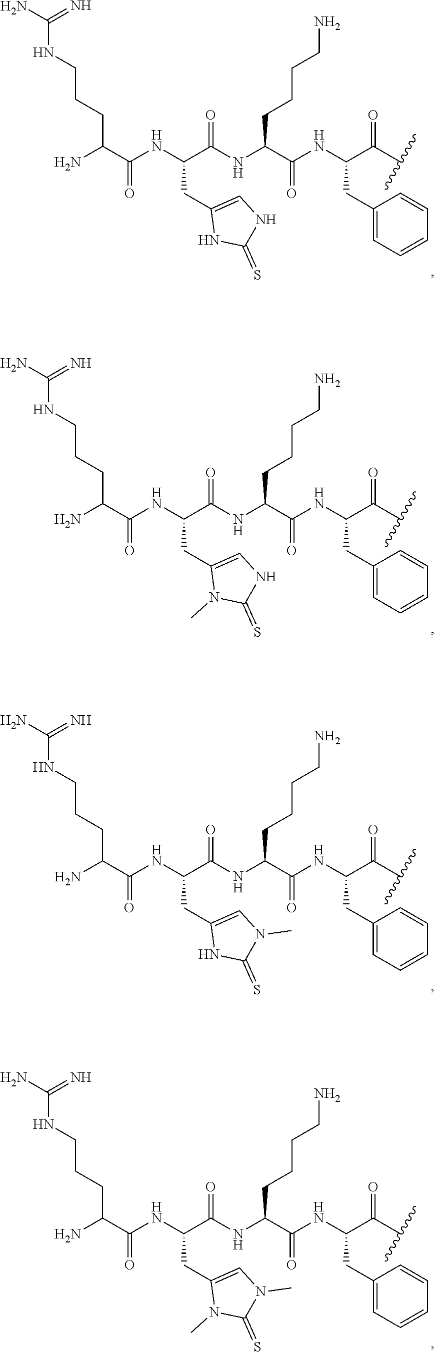 [SMILES: *C(=O)[C@H](Cc1ccccc1)NC(=O)[C@H](CCCCN)NC(=O)[C@H](Cc1c[nH]c(=S)[nH]1)NC(=O)C(N)CCCNC(=N)N ; *C(=O)[C@H](Cc1ccccc1)NC(=O)[C@H](CCCCN)NC(=O)[C@H](Cc1c[nH]c(=S)n1C)NC(=O)C(N)CCCNC(=N)N ; *C(=O)[C@H](Cc1ccccc1)NC(=O)[C@H](CCCCN)NC(=O)[C@H](Cc1cn(C)c(=S)[nH]1)NC(=O)C(N)CCCNC(=N)N ; *C(=O)[C@H](Cc1ccccc1)NC(=O)[C@H](CCCCN)NC(=O)[C@H](Cc1cn(C)c(=S)n1C)NC(=O)C(N)CCCNC(=N)N]

or a protonated analogue thereof.
Statement 5. A peptide or protein according to Statement 1, wherein the peptide or protein comprises the following sequence:

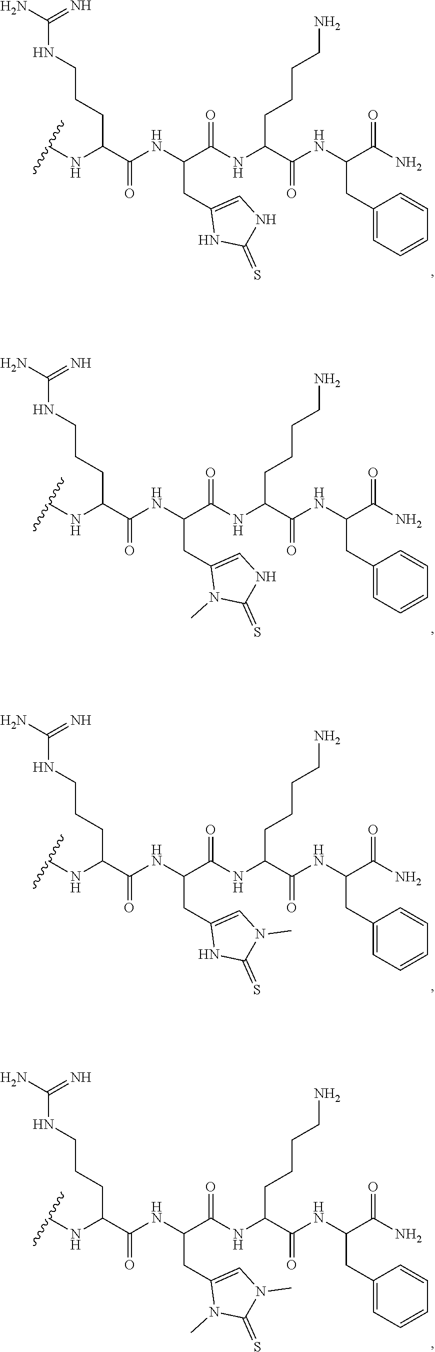 [SMILES: *NC(CCCNC(=N)N)C(=O)NC(Cc1c[nH]c(=S)[nH]1)C(=O)NC(CCCCN)C(=O)NC(Cc1ccccc1)C(N)=O ; *NC(CCCNC(=N)N)C(=O)NC(Cc1c[nH]c(=S)n1C)C(=O)NC(CCCCN)C(=O)NC(Cc1ccccc1)C(N)=O ; *NC(CCCNC(=N)N)C(=O)NC(Cc1cn(C)c(=S)[nH]1)C(=O)NC(CCCCN)C(=O)NC(Cc1ccccc1)C(N)=O ; *NC(CCCNC(=N)N)C(=O)NC(Cc1cn(C)c(=S)n1C)C(=O)NC(CCCCN)C(=O)NC(Cc1ccccc1)C(N)=O]

or a protonated analogue thereof.
Statement 6. A peptide or protein according to Statement 5, wherein the peptide or protein comprises the following sequence:

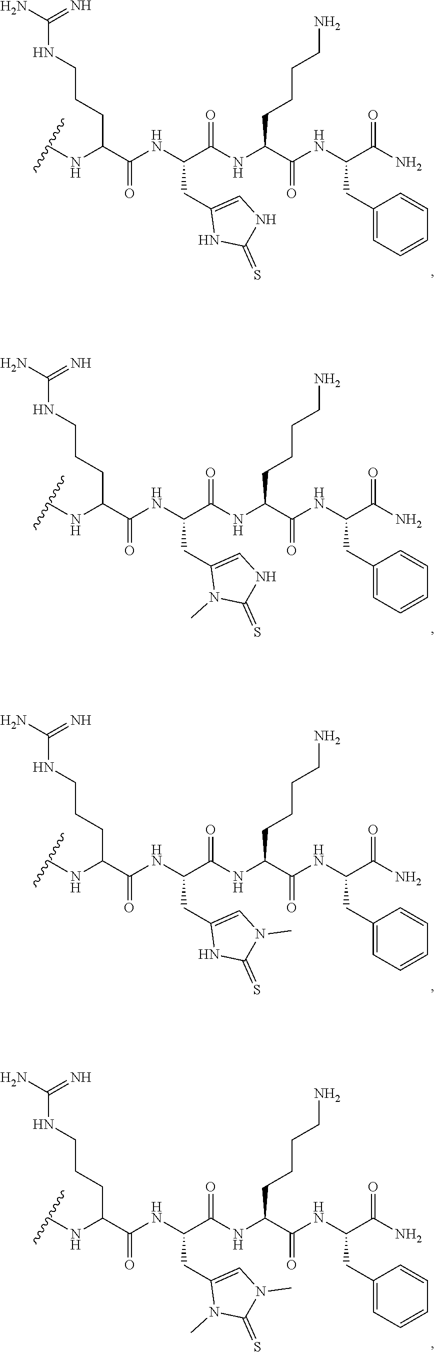 [SMILES: *NC(CCCNC(=N)N)C(=O)N[C@@H](Cc1c[nH]c(=S)[nH]1)C(=O)N[C@@H](CCCCN)C(=O)N[C@@H](Cc1ccccc1)C(N)=O ; *NC(CCCNC(=N)N)C(=O)N[C@@H](Cc1c[nH]c(=S)n1C)C(=O)N[C@@H](CCCCN)C(=O)N[C@@H](Cc1ccccc1)C(N)=O ; *NC(CCCNC(=N)N)C(=O)N[C@@H](Cc1cn(C)c(=S)[nH]1)C(=O)N[C@@H](CCCCN)C(=O)N[C@@H](Cc1ccccc1)C(N)=O ; *NC(CCCNC(=N)N)C(=O)N[C@@H](Cc1cn(C)c(=S)n1C)C(=O)N[C@@H](CCCCN)C(=O)N[C@@H](Cc1ccccc1)C(N)=O]

or a protonated analogue thereof.
Statement 7. A peptide or protein according to Statement 1, wherein the peptide or protein comprises the following sequence:

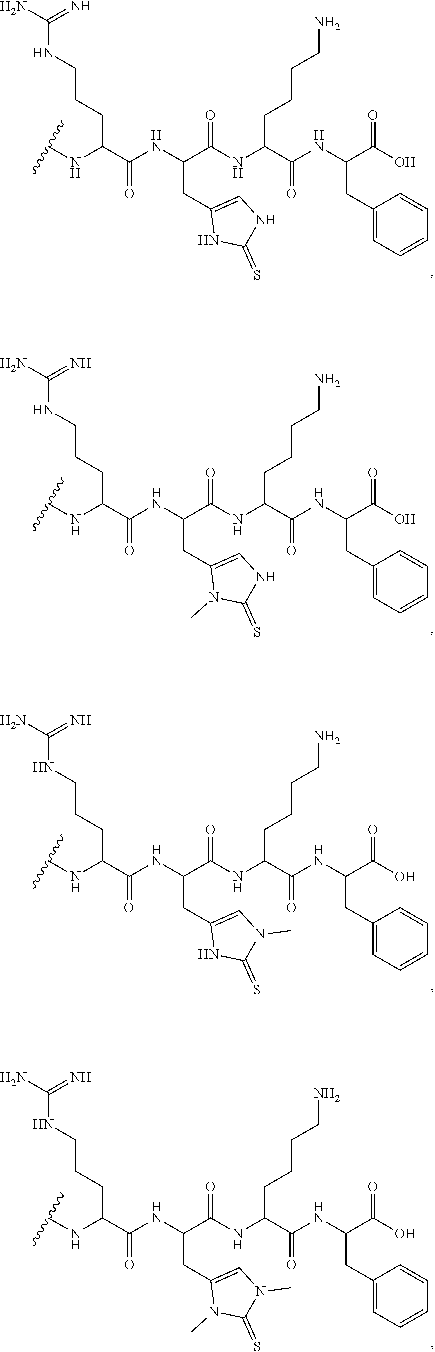 [SMILES: *NC(CCCNC(=N)N)C(=O)NC(Cc1c[nH]c(=S)[nH]1)C(=O)NC(CCCCN)C(=O)NC(Cc1ccccc1)C(=O)O ; *NC(CCCNC(=N)N)C(=O)NC(Cc1c[nH]c(=S)n1C)C(=O)NC(CCCCN)C(=O)NC(Cc1ccccc1)C(=O)O ; *NC(CCCNC(=N)N)C(=O)NC(Cc1cn(C)c(=S)[nH]1)C(=O)NC(CCCCN)C(=O)NC(Cc1ccccc1)C(=O)O ; *NC(CCCNC(=N)N)C(=O)NC(Cc1cn(C)c(=S)n1C)C(=O)NC(CCCCN)C(=O)NC(Cc1ccccc1)C(=O)O]

or a protonated or deprotonated analogue thereof.
Statement 8. A peptide or protein according to Statement 7, wherein the peptide or protein comprises the following sequence:

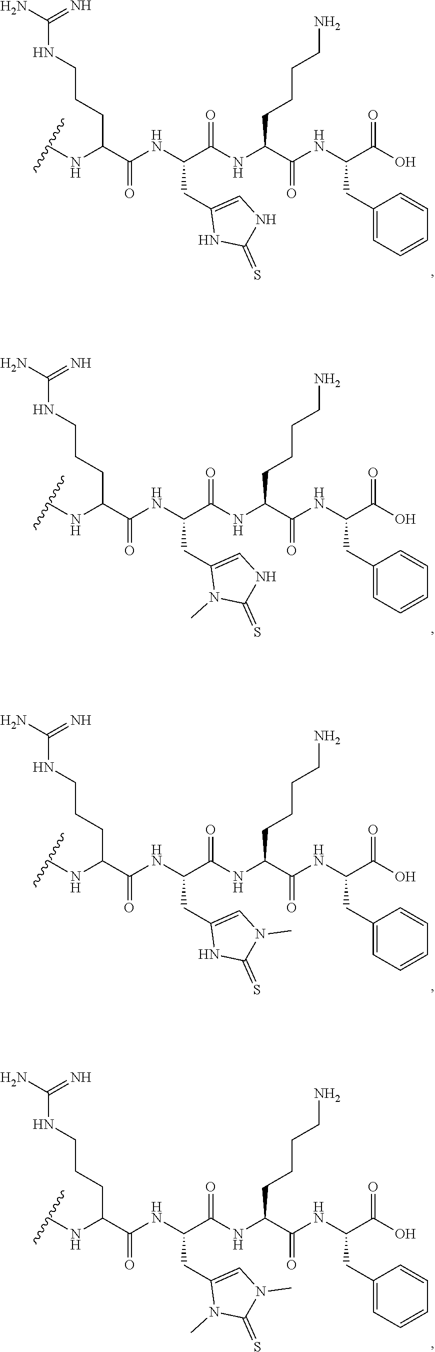 [SMILES: *NC(CCCNC(=N)N)C(=O)N[C@@H](Cc1c[nH]c(=S)[nH]1)C(=O)N[C@@H](CCCCN)C(=O)N[C@@H](Cc1ccccc1)C(=O)O ; *NC(CCCNC(=N)N)C(=O)N[C@@H](Cc1c[nH]c(=S)n1C)C(=O)N[C@@H](CCCCN)C(=O)N[C@@H](Cc1ccccc1)C(=O)O ; *NC(CCCNC(=N)N)C(=O)N[C@@H](Cc1cn(C)c(=S)[nH]1)C(=O)N[C@@H](CCCCN)C(=O)N[C@@H](Cc1ccccc1)C(=O)O ; *NC(CCCNC(=N)N)C(=O)N[C@@H](Cc1cn(C)c(=S)n1C)C(=O)N[C@@H](CCCCN)C(=O)N[C@@H](Cc1ccccc1)C(=O)O]

or a protonated or deprotonated analogue thereof.
Statement 9. A peptide or protein according to Statement 1, wherein the peptide or protein has the following sequence:

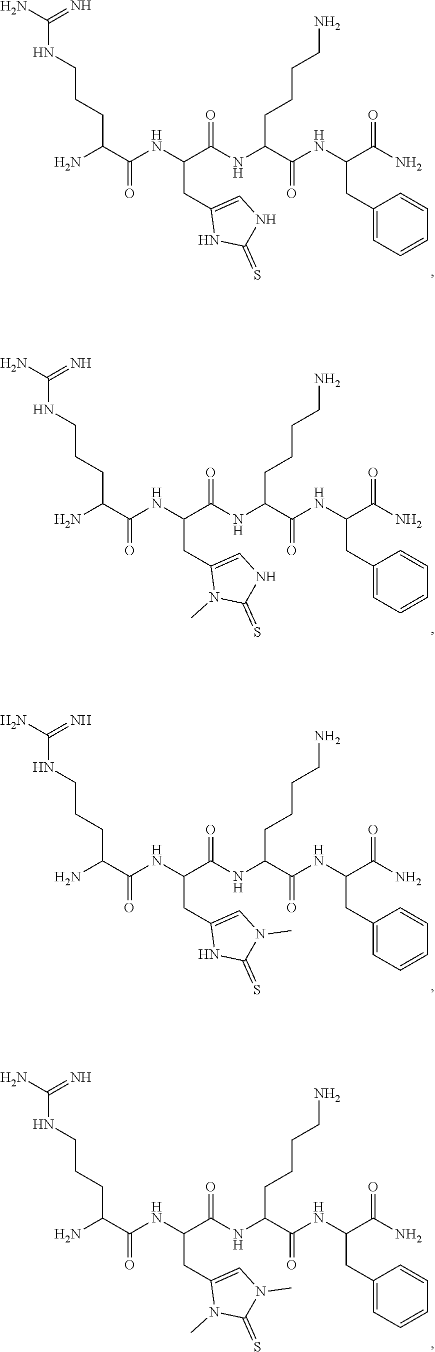 [SMILES: N=C(N)NCCCC(N)C(=O)NC(Cc1c[nH]c(=S)[nH]1)C(=O)NC(CCCCN)C(=O)NC(Cc1ccccc1)C(N)=O ; Cn1c(CC(NC(=O)C(N)CCCNC(=N)N)C(=O)NC(CCCCN)C(=O)NC(Cc2ccccc2)C(N)=O)c[nH]c1=S ; Cn1cc(CC(NC(=O)C(N)CCCNC(=N)N)C(=O)NC(CCCCN)C(=O)NC(Cc2ccccc2)C(N)=O)[nH]c1=S ; Cn1cc(CC(NC(=O)C(N)CCCNC(=N)N)C(=O)NC(CCCCN)C(=O)NC(Cc2ccccc2)C(N)=O)n(C)c1=S]

or a protonated analogue thereof.
Statement 10. A peptide or protein according to Statement 9, wherein the peptide or protein has the following sequence:

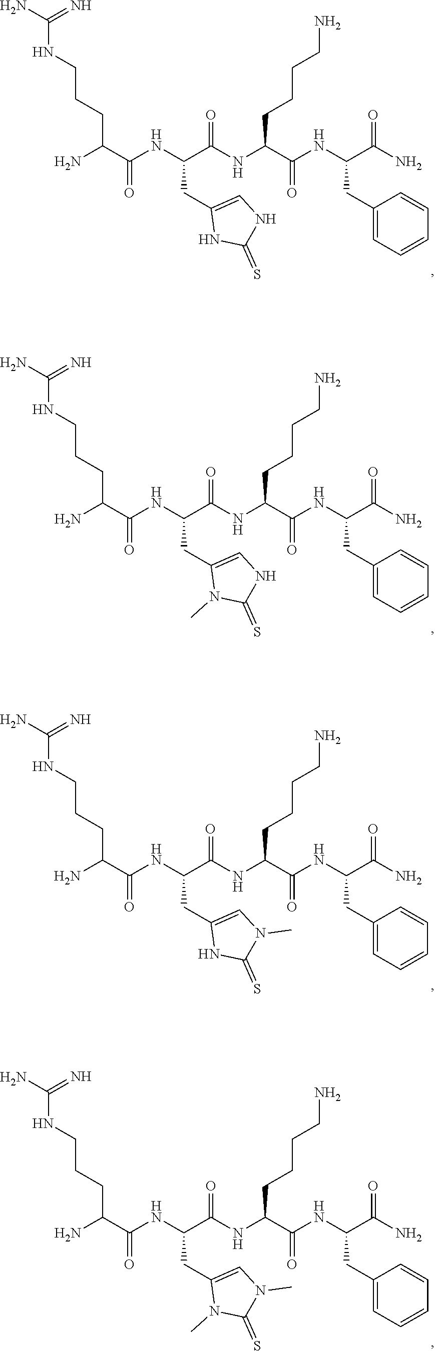 [SMILES: N=C(N)NCCCC(N)C(=O)N[C@@H](Cc1c[nH]c(=S)[nH]1)C(=O)N[C@@H](CCCCN)C(=O)N[C@@H](Cc1ccccc1)C(N)=O ; Cn1c(C[C@H](NC(=O)C(N)CCCNC(=N)N)C(=O)N[C@@H](CCCCN)C(=O)N[C@@H](Cc2ccccc2)C(N)=O)c[nH]c1=S ; Cn1cc(C[C@H](NC(=O)C(N)CCCNC(=N)N)C(=O)N[C@@H](CCCCN)C(=O)N[C@@H](Cc2ccccc2)C(N)=O)[nH]c1=S ; Cn1cc(C[C@H](NC(=O)C(N)CCCNC(=N)N)C(=O)N[C@@H](CCCCN)C(=O)N[C@@H](Cc2ccccc2)C(N)=O)n(C)c1=S]

or a protonated analogue thereof.
Statement 11. A peptide or protein according to Statement 1, wherein the peptide or protein has the following sequence:

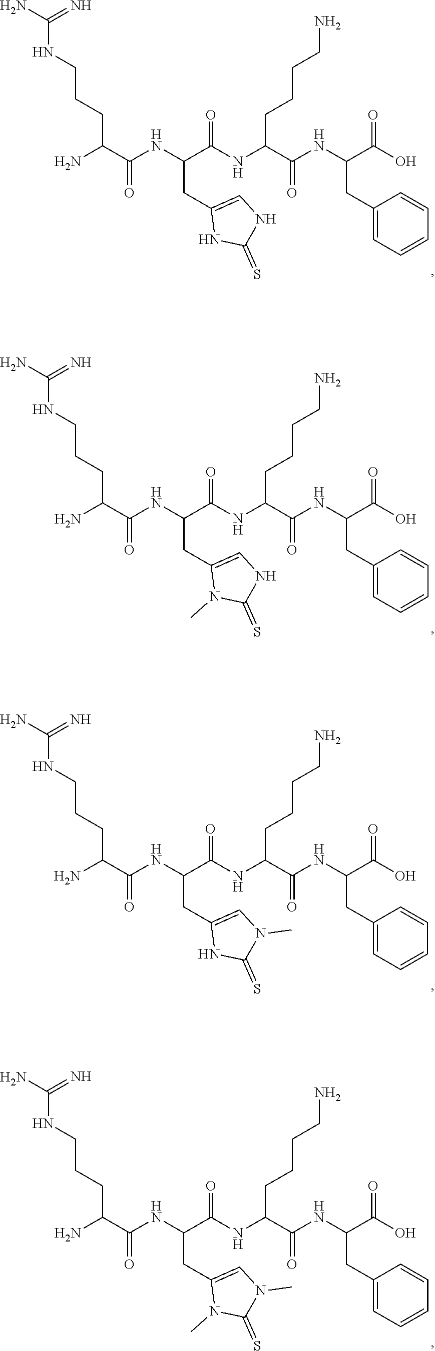 [SMILES: N=C(N)NCCCC(N)C(=O)NC(Cc1c[nH]c(=S)[nH]1)C(=O)NC(CCCCN)C(=O)NC(Cc1ccccc1)C(=O)O ; Cn1c(CC(NC(=O)C(N)CCCNC(=N)N)C(=O)NC(CCCCN)C(=O)NC(Cc2ccccc2)C(=O)O)c[nH]c1=S ; Cn1cc(CC(NC(=O)C(N)CCCNC(=N)N)C(=O)NC(CCCCN)C(=O)NC(Cc2ccccc2)C(=O)O)[nH]c1=S ; Cn1cc(CC(NC(=O)C(N)CCCNC(=N)N)C(=O)NC(CCCCN)C(=O)NC(Cc2ccccc2)C(=O)O)n(C)c1=S]

or a protonated or deprotonated analogue thereof.
Statement 12. A peptide or protein according to Statement 11, wherein the peptide or protein has the following sequence:

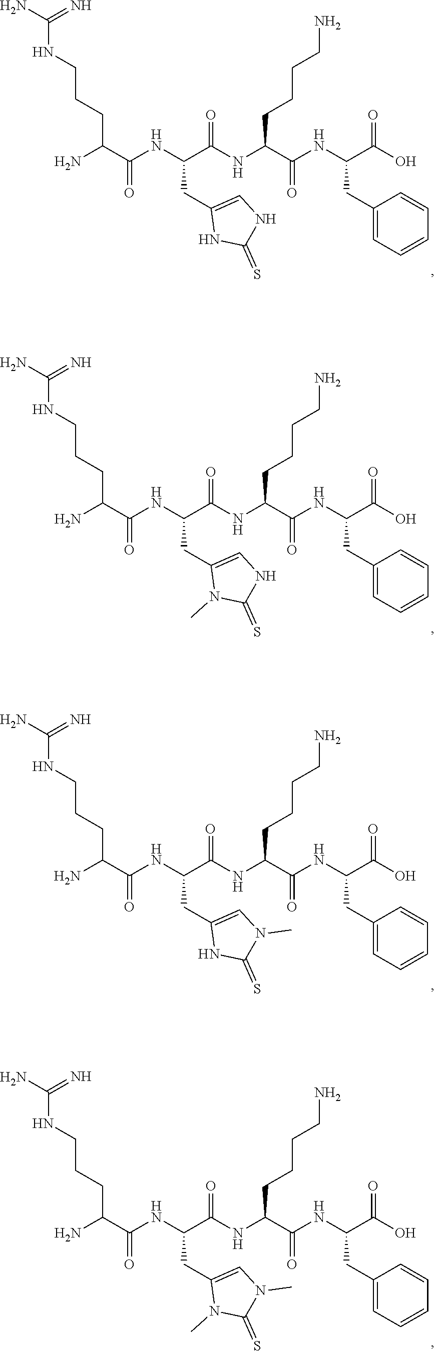 [SMILES: N=C(N)NCCCC(N)C(=O)N[C@@H](Cc1c[nH]c(=S)[nH]1)C(=O)N[C@@H](CCCCN)C(=O)N[C@@H](Cc1ccccc1)C(=O)O ; Cn1c(C[C@H](NC(=O)C(N)CCCNC(=N)N)C(=O)N[C@@H](CCCCN)C(=O)N[C@@H](Cc2ccccc2)C(=O)O)c[nH]c1=S ; Cn1cc(C[C@H](NC(=O)C(N)CCCNC(=N)N)C(=O)N[C@@H](CCCCN)C(=O)N[C@@H](Cc2ccccc2)C(=O)O)[nH]c1=S ; Cn1cc(C[C@H](NC(=O)C(N)CCCNC(=N)N)C(=O)N[C@@H](CCCCN)C(=O)N[C@@H](Cc2ccccc2)C(=O)O)n(C)c1=S]

or a protonated or deprotonated analogue thereof.
Statement 12a. A peptide or protein according to Statement 1, wherein the peptide has the following structure:

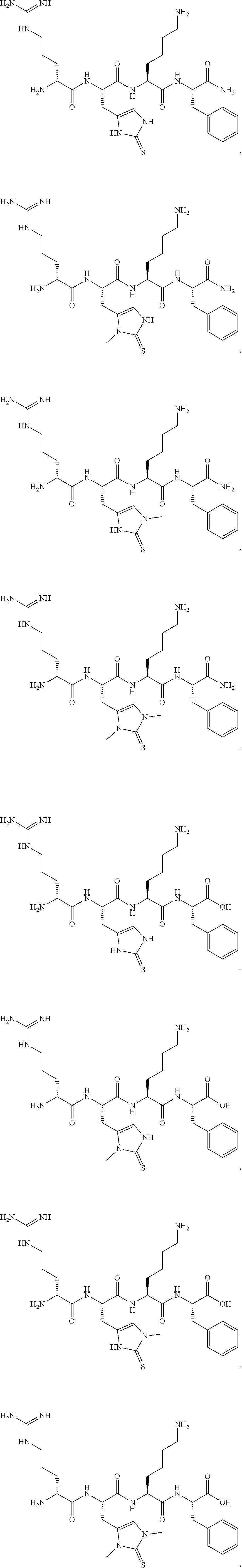

or a protonated or deprotonated analogue thereof.
Statement 13. A composition comprising a peptide or protein according to any one of the preceding Statements.
Statement 14. A composition according to Statement 13, wherein the composition further comprises a pharmaceutically acceptable carrier.
Statement 15. A method for treating an individual having or suspected of having a disease associated with mitochondrial dysfunction or an individual having or suspected of having chlorine gas exposure, comprising administering to the individual a composition according to Statement 13 or Statement 14.
Statement 16. A method according to Statement 15, wherein the disease is Duchenne's muscular dystrophy, age-related macular degeneration, Barth syndrome, Leber's hereditary optic neuropathy, or a combination thereof.
Statement 17. A kit comprising a peptide or protein according to any one of Statements 1-12a and instructions for use.
Statement 18. A kit according to Statement 17, wherein the kit further comprises a diluent or pharmaceutically acceptable carrier.
Statement 19. A kit according to Statement 17 or Statement 18, wherein the peptide or protein is provided as a lyophilized powder.
Statement 20. A kit according to Statement 17 or Statement 18, wherein the peptide or protein is provided dissolved a solvent.

The following example is presented to illustrate the present disclosure. It is not intended to be limiting in any matter.

### Example 1

This example provides a description of methods, peptides, and uses of peptides of the present disclosure.

An analogue of the peptide known as “SS-31” was synthesized. The “SS” is for Szeto-Shiller peptides. The sequence of SS-31 is D-Arg-dimethyl-tyrosine-Lys-Phe-amide. This analogue replaces the antioxidant amino acid dimethyl-tyrosine with 2-thiohistidine. These in vitro data shows that this analogue is a significantly better antioxidant than the SS-31 peptide. Some of these cell-based assays also support that the analogue has higher antioxidant activity. SS-31 is known by its trade name “Elamipretide” and is being studied in various clinical trials. SS-31 targets the mitochondria, the main source of oxidative stress in the cell. The clinical trials hope to show that SS-31 can be used to treat various diseases where mitochondrial dysfunction plays an important role such as Duchenne's muscular dystrophy, dry age-related macular degeneration, Barth syndrome, and Leber's hereditary optic neuropathy.

Various in vitro antioxidant capacity studies and some cell-based protection assays have been performed. These studies demonstrate that our analogue has significant advantages over SS-31. This is especially true for HOCl-mediated injury as illustrated in FIG. 9.

Synthesis of L-2-thio-histidine. L-2-thiohistidine was synthesized according to a known procedure. This reaction works best when performed on 10 g or higher scale. Histidine (14 g, 66.8 mmol, 1.0 eq.) was dissolved in 134 mL of deionized water. After the His was fully dissolved, this solution was cooled in an ice bath at 0° C. Once the reaction was cooled, bromine (4.45 mL, 86.8 mmol, 1.3 eq.) was added resulting in a bright orange solution. After 6 min, Cys (24.3 g, 200.4 mmol, 3.0 eq.) was added to the reaction. The solution was stirred at 0° C. for 1 h. An oil bath was preheated to 95° C. After 1 h, 3-mercaptopropionic acid (34.9 mL, 400.7 mmol, 6.0 eq.) was added to the reaction and the reaction was transferred to the oil bath at 95° C. A condenser was attached to the reaction flask and the reaction was stirred for 18 h at 95° C., after which the reaction had turned dark brown. The reaction flask was removed from the oil bath and condenser was removed and the reaction flask was allowed to cool to room temperature. The aqueous solution was then extracted with ethyl acetate. The aqueous layer remained dark brown after extraction. The aqueous layer was transferred to a clean flask and placed in an oil bath preheated to 40° C. The pH of the solution was adjusted to 6.5 with 30% ammonia hydroxide to precipitate 2-thioHis. The reaction was chilled to allow complete precipitation. The off-white precipitate was filtered out of the reaction and washed with cold deionized water and ethanol. The precipitate was dried under high vacuum to give 5.04 g (26.9 mmol) of an off-white powder. The percent yield of this reaction was 40% which is consistent with known findings. Mass spectrometric (MS) analysis revealed a peak at 188.1 m/z. ¹H-NMR (D₂O/DCl): δ 3.06-3.20 ((2H, (3.06 dd) (3.20 (dd)), 4.21 (1H, dd), 6.79 (1H, s); ¹³C-NMR (D₂O/DCl): δ 25.32, 51.82, 115.96, 123.23, 156.49, 170.38.

Addition of Fmoc protecting group to L-2-thioHis. N-Fmoc-L-2-thioHis was prepared using a standard procedure for the addition of fluorenylmethoxycarbonyl (Fmoc) protecting groups to amino acids. In a 250 mL round bottom flask, 2-thioHis (1.0 g) was added to 5-10 mL of deionized water to create a slurry. Triethylamine (TEA) (750 μL, 5.35 mmol, 1.0 eq.) was added to the amino acid slurry, and the reaction was stirred at room temperature. Fmoc N-hydroxysuccinimide ester (Fmoc-OSu) (1.99 g, 5.89 mmol, 1.1 eq.) was dissolved in 20-30 mL of acetonitrile and added to the amino acid slurry. A second eq. of TEA (750 μL) was added to the reaction along with acetonitrile and water to completely dissolve the 2-thioHis. The reaction was stirred for 2 h at room temperature and monitored by thin-layer chromatography (TLC). The reaction was quenched by acidifying with 20 mL of 1 N HCl. The reaction was extracted 3× with ethyl acetate followed by a back extraction of the ethyl acetate layer with water, 1 N HCl, and brine (1:1:1). The ethyl acetate solution was then dried with MgSO₄, filtered with a Büchner funnel, and roto-evaporated to dryness. The oil was dissolved in 10-20 mL of ethyl acetate with 1-2 mL of methanol. The addition of hexanes precipitated the N-Fmoc-2-thioHis derivative as a cream colored solid. The solid was purified by redissolving it in 10-20 mL of warm ethyl acetate and 1-2 mL of methanol, filtering the solution through a Büchner funnel, and then reprecipitating the product with cold hexanes. The product was dried under high vacuum and used without further purification. Mass spectrometric analysis showed a dominant peak at 410 m/z for the product as well as smaller peaks for the Na⁺ adduct (M+23) at m/z 432 and the K⁺ adduct (M+39) at m/z 448. ¹H-NMR (MeOD): δ 2.88 (dd, 1H), 3.08 (dd, 1H), 4.22 (t, 1H), 4.35 (d, 2H), 4.43 dd, 1H), 6.60 (s, 1H), 7.32 (t, 2H), 7.40 (t, 2H), 7.65 (d, 2H), 7.80 (d, 2H); ¹³C-NMR (MeOD) δ 26.77, 53.06, 66.61, 119.49, 124.81, 126.78, 127.38, 141.16, 143.76, 143.83, 157.00, 172.87. An average yield was 82.5%.

Peptide synthesis. All His-containing peptides were synthesized according to standard SPPS protocols on a 0.1-mmol scale using a glass vessel shaken with a model 75 Burrell wrist action shaker. Peptides were cleaved from the resin with a cleavage cocktail consisting of trifluoroacetic acid (TFA)/triisopropylsilane (TIS)/water (96:2:2) for 1.5 h. Following cleavage, the resin was washed with DCM, and the volume of the cleavage solution was reduced by evaporation with argon gas. Each peptide was precipitated with cold, anhydrous ether. Centrifugation at 3000 rpm on a clinical centrifuge (International Equipment Co, Boston, MA) for 5 min pelleted the peptide. Peptides were dried under argon gas, then dissolved in a minimal amount of water/HPLC-grade acetonitrile (5:1), lyophilized, and used without further purification.

SS-31 is active at the inner mitochondrial membrane, where it interacts with the anionic phospholipid cardiolipin. Cardiolipin is important for folding of mitochondrial cristae (the folds of the inner membrane). The spatial coordination of cytochrome c with the rest of the proteins in the electron-transport chain.

Experiments were performed to determine DPPH antioxidant activity. Radical concentration of DPPH was determined after reacting with varying concentrations of peptide. DPPH is an organic radical with a peak ABS at 525 nm. The quenched version of the radical does not absorb 525 nm light. FIG. 3 shows the results of these experiments.

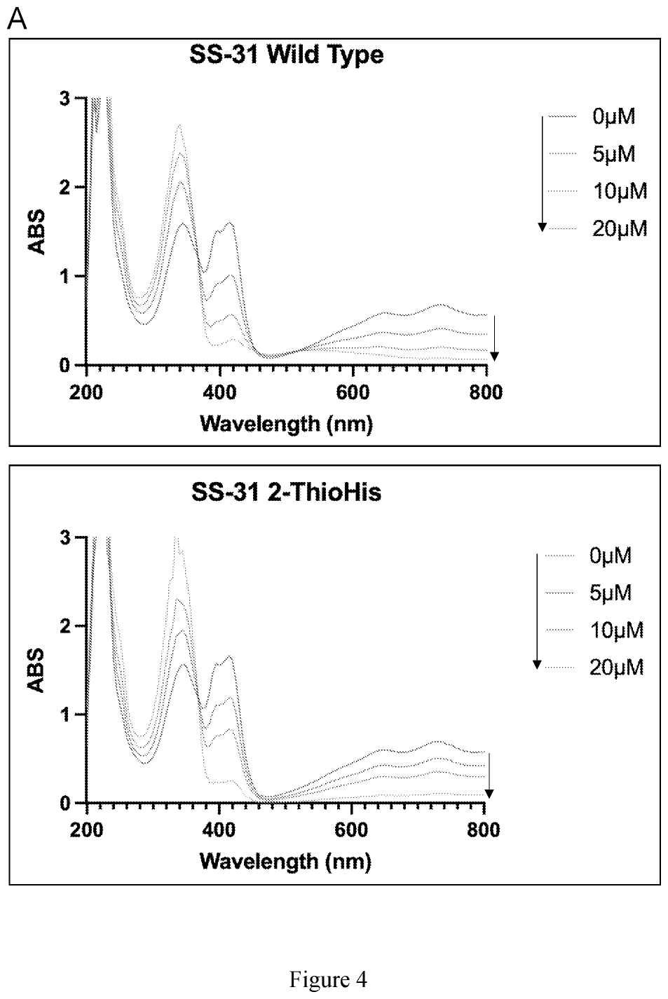

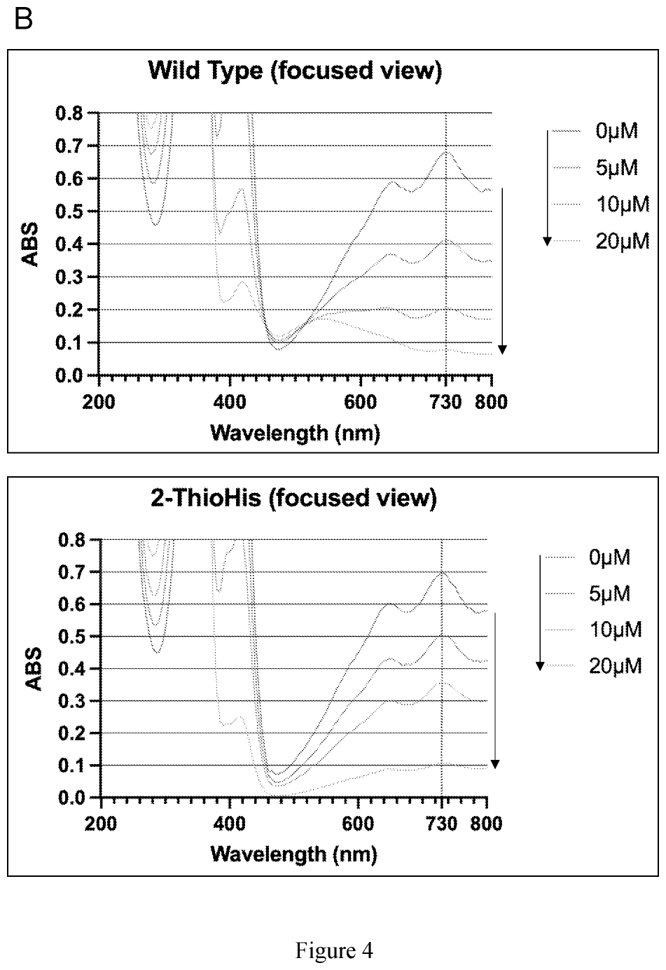

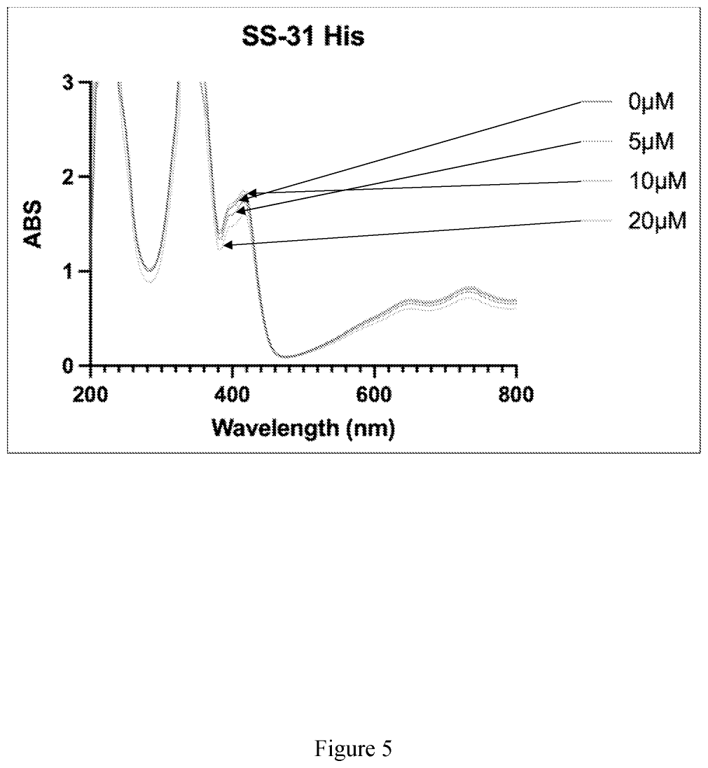

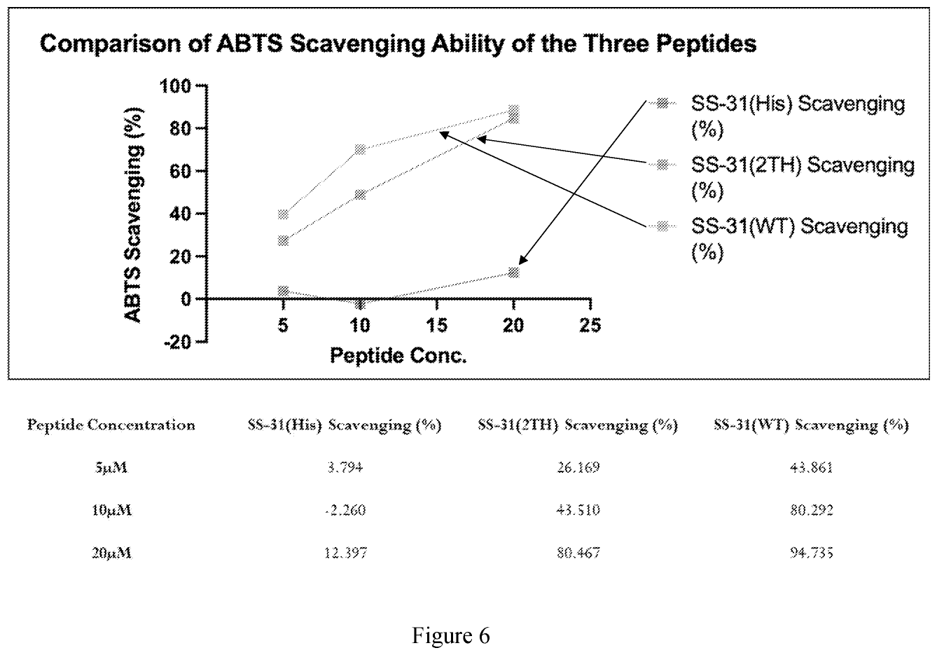

ABTS assays were also performed. ABTS is a radical cation that is blue in color and absorbs light at 734 nm. 7 mM AzBTS was mixed in water with 2.45 mM potassium persulfate and left 16 hrs in the dark at rt to oxidize. ABTS and potassium persulfate react stoichiometrically at a ratio of 1:0.5, which resulted in incomplete oxidation of ABTS. The ABTS solution was diluted to an absorbance of ˜0.7 at 734 nm. Samples were assayed with ABTS: 990 μL ABTS solution+10 μL sample. Stocks of peptides were made with concentrations in the range of 0.1 mM to 3.2 mM. This assay may be a better choice than DPPH if there is less steric hindrance. FIGS. 4-6 show the results of these experiments.

Hydroxyl radical scavenging activity of peptides measured by electron paramagnetic resonance (EPR). Hydroxyl radical was generated by a reaction of 0.8 mM FeSO4, with 0.8-mM H₂O₂, pH 7.0 (250 μL). The hydroxyl radicals were trapped with 5,5-dimethyl-1-pyrroline-N-oxide (DMPO) prior to obtaining spectra. These data are shown in FIG. 7.

An ROS glow assay was also performed. The assay measures the levels of H₂O₂ after injury to the cell. Human HMESO malignant mesothelioma cells were incubated with 250 mM of WT, 2-TH or His compound with the addition 50 μM menadione and the H₂O₂ substrate for 4 hours (in duplicate). The reaction was quenched with addition of the H₂O₂ detection substrate. Luminescence levels were measured in a plate reader after 30 minutes of incubation. These data are shown in FIG. 8.

An HOCl protection assay using 16HBE41o-cells was performed. This assay measured protection against cell death using HOCl as the oxidant. RH8=2TH peptide and RH9=His peptide. The WT peptide did not show any protection in this assay. These data are shown in FIG. 9. For this assay, 16HBE41o-cells were treated with 200 μM of HOCl for 60 minutes in PBS with or without increasing concentrations of test compounds. Cells were washed and then placed back in media and cell viability was assess at 24 hours using the MTT assay. % Protection was determined by normalizing data to cell viability with and without oxidant. Data curves were generated using Prism 8 software (GraphPad) for 50% effective concentration (EC₅₀) determinations and their respective 95% confidence intervals.

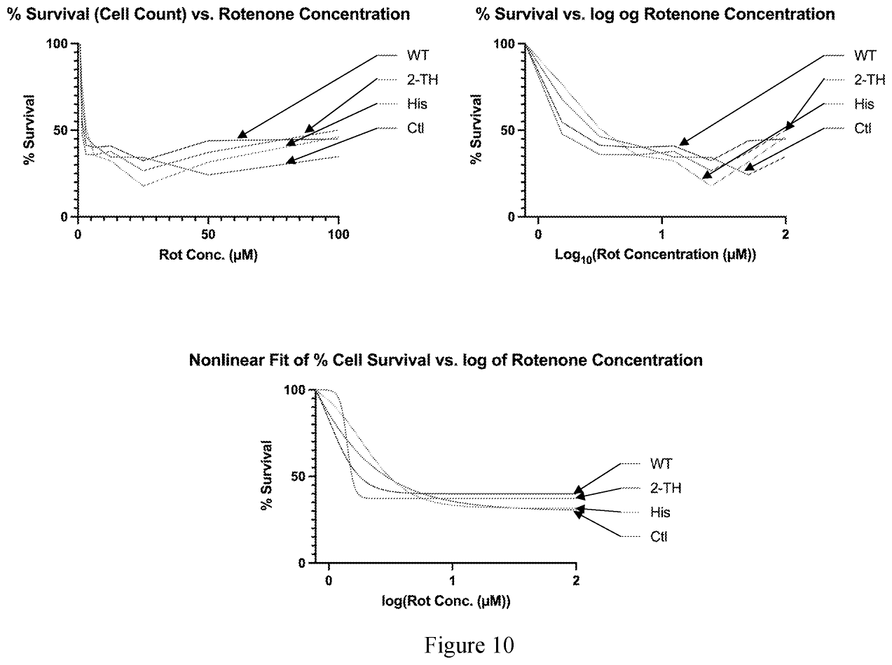

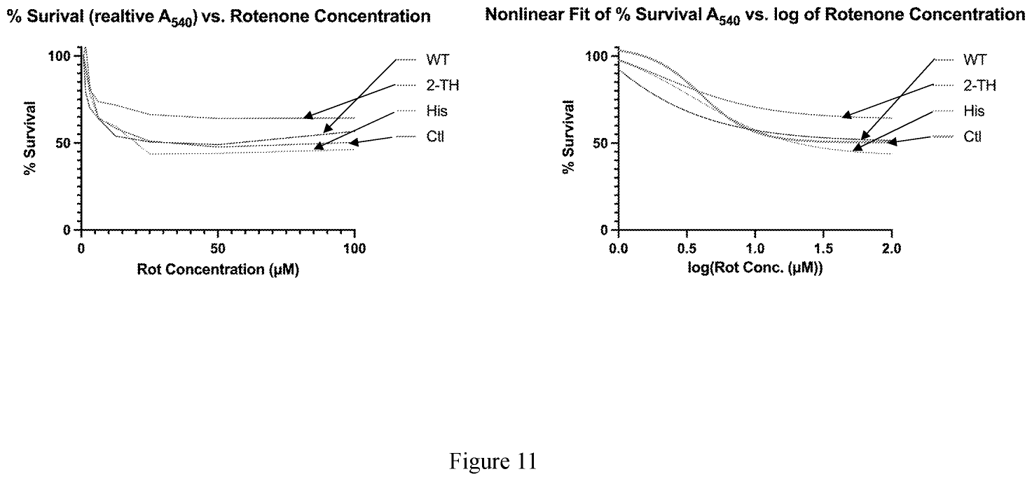

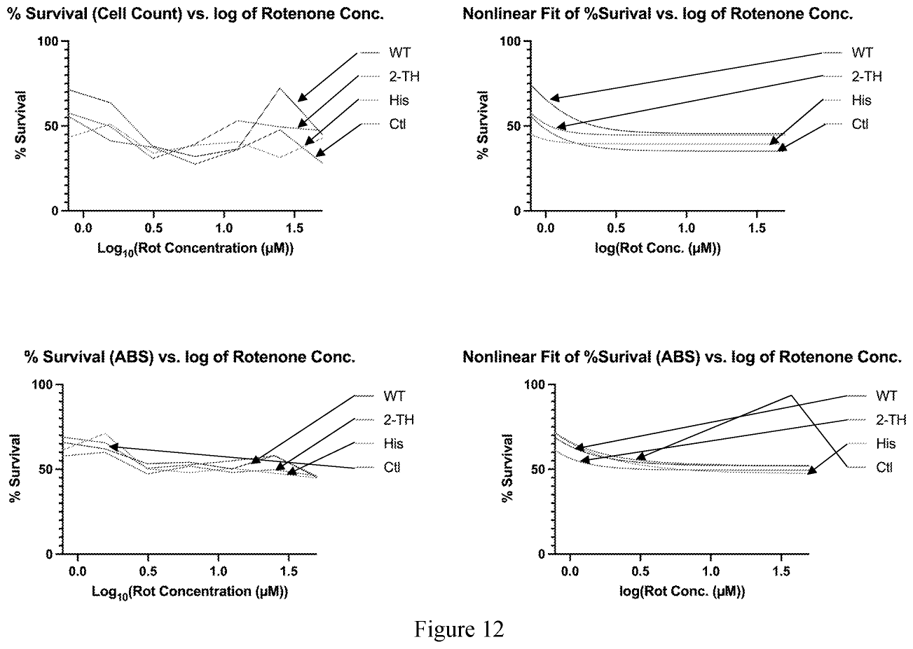

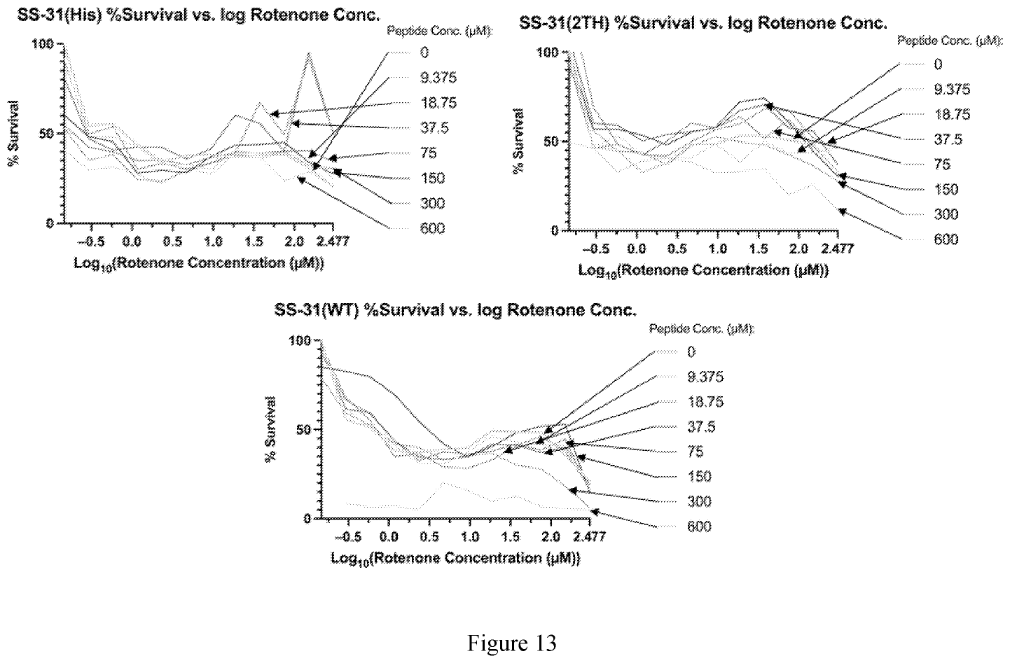

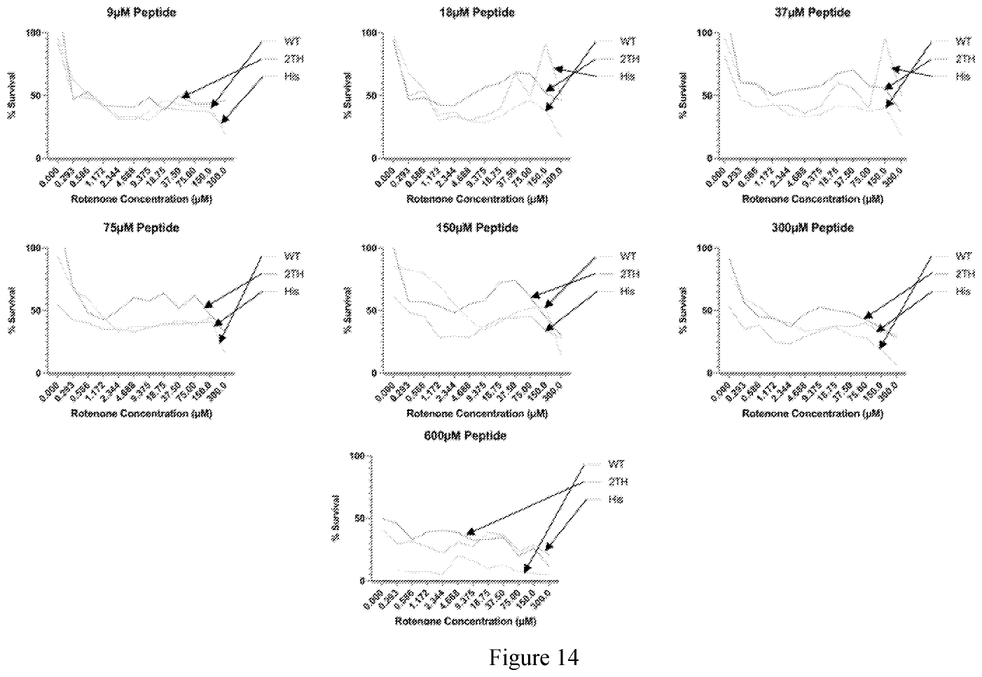

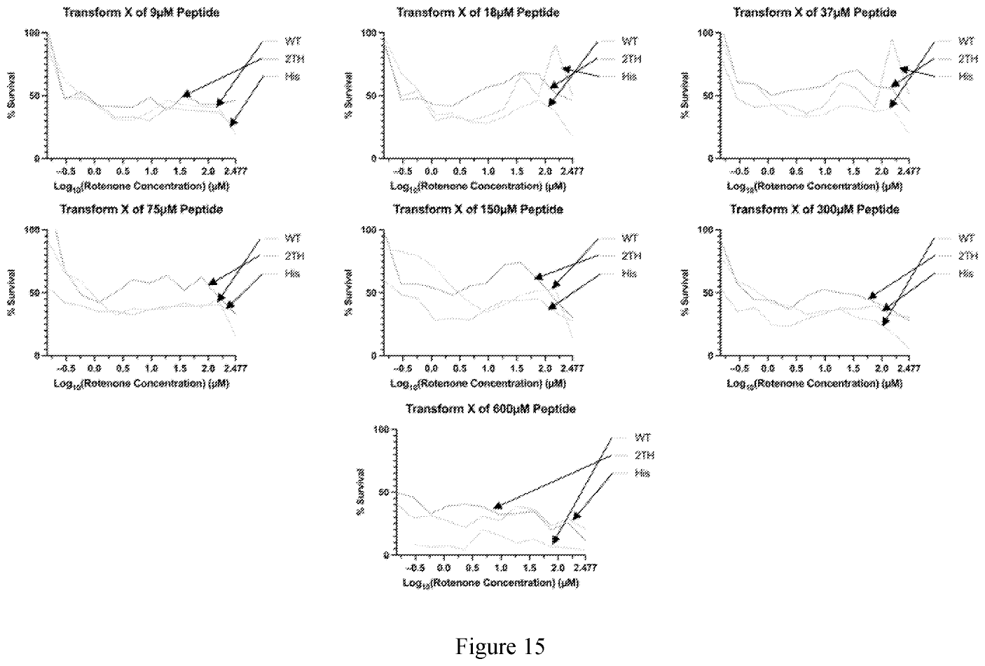

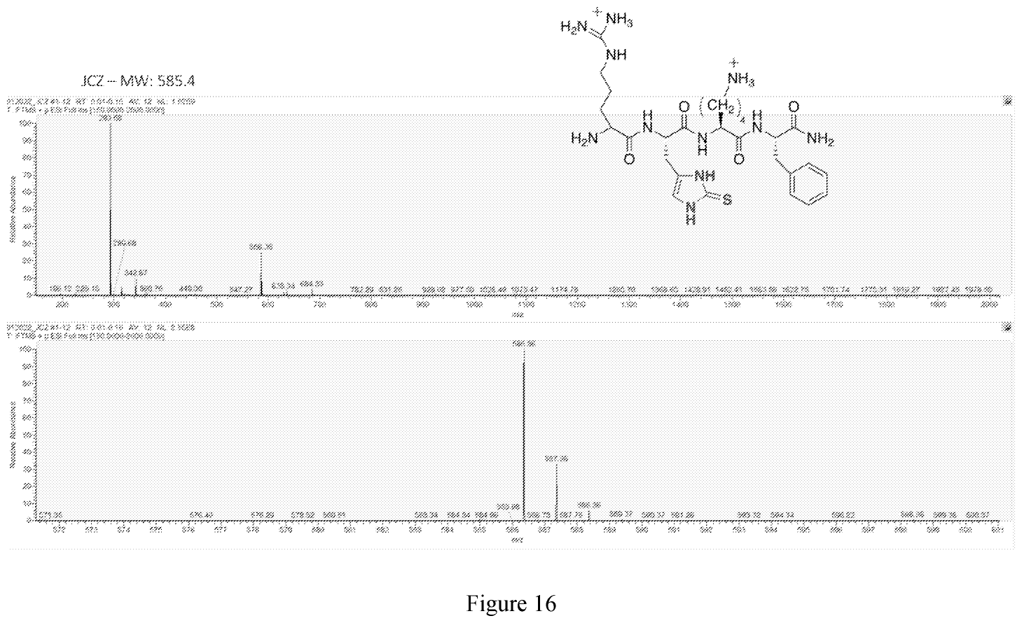

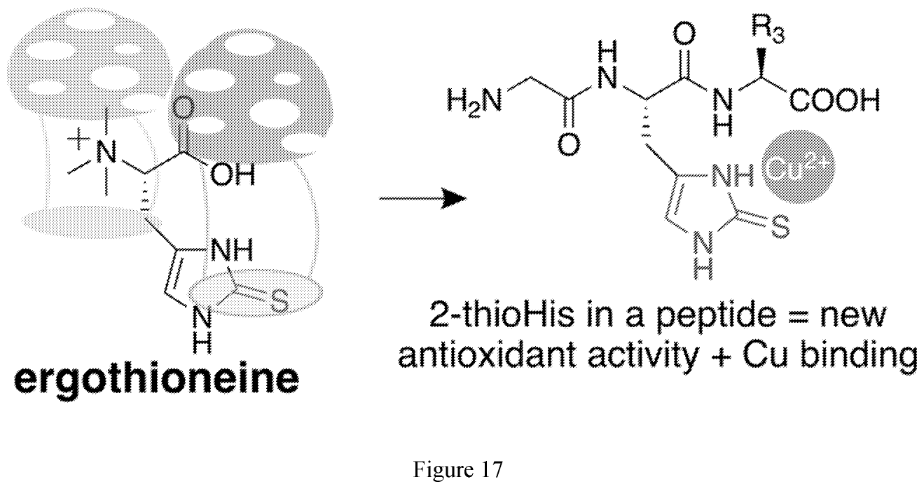

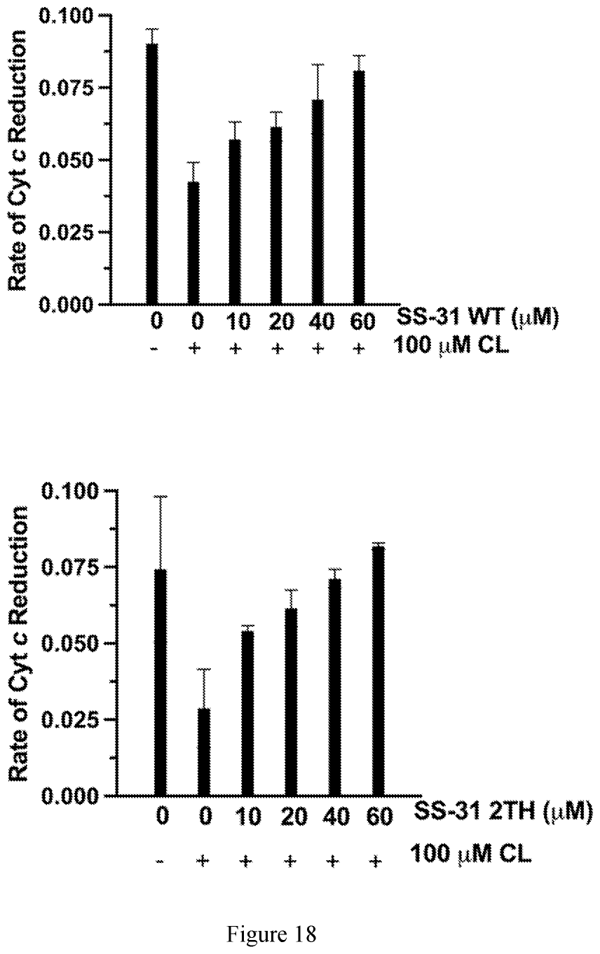

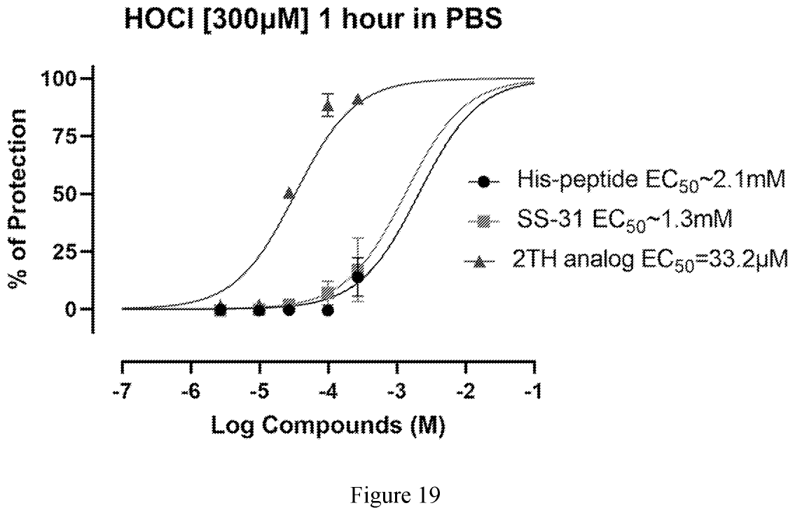

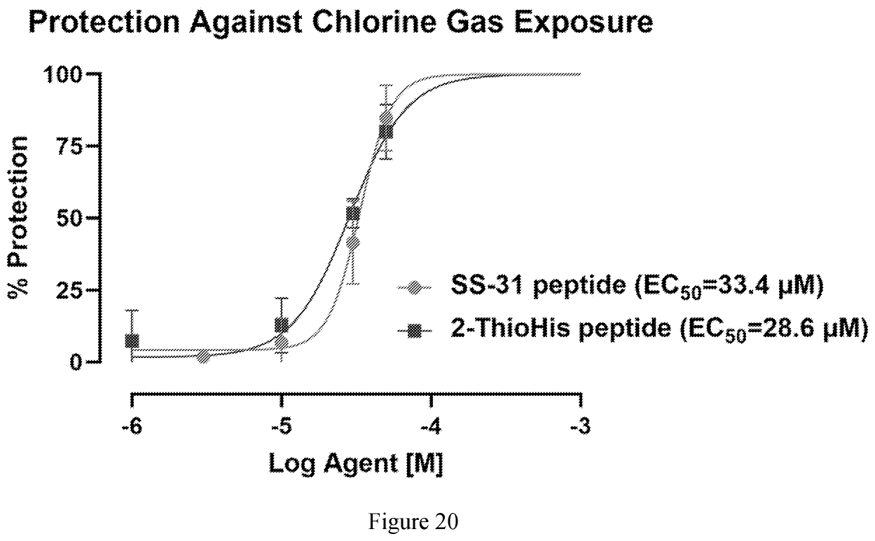

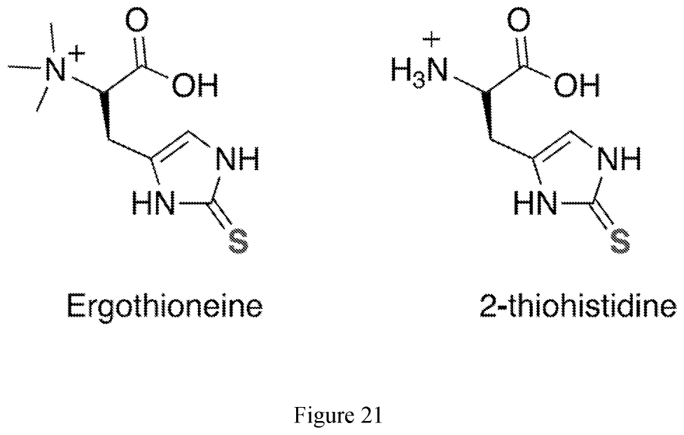

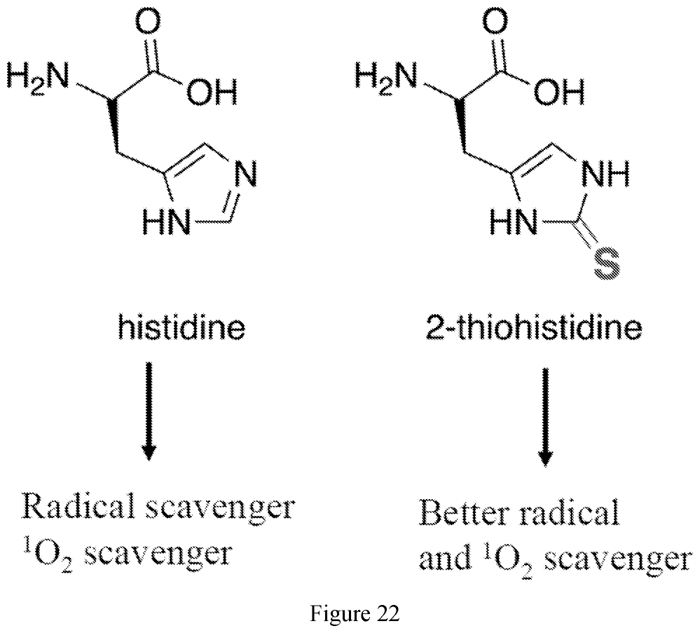

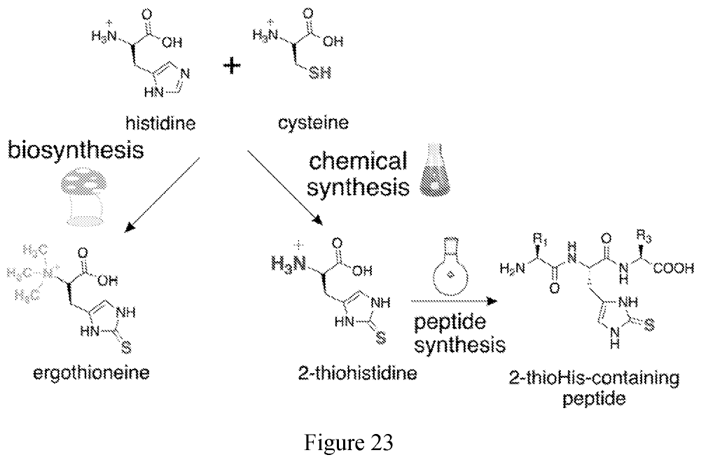

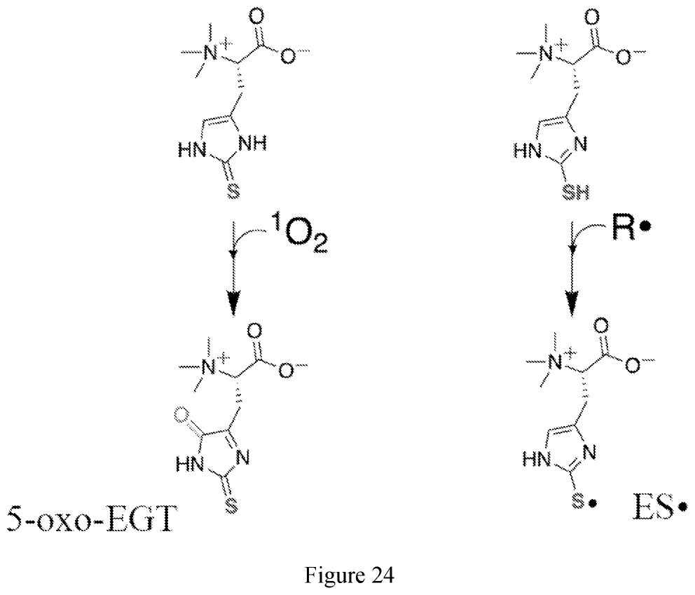

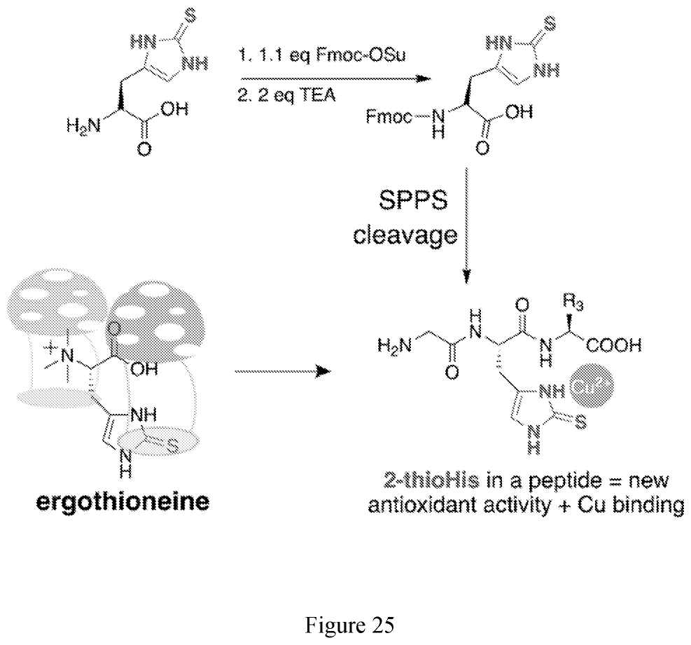

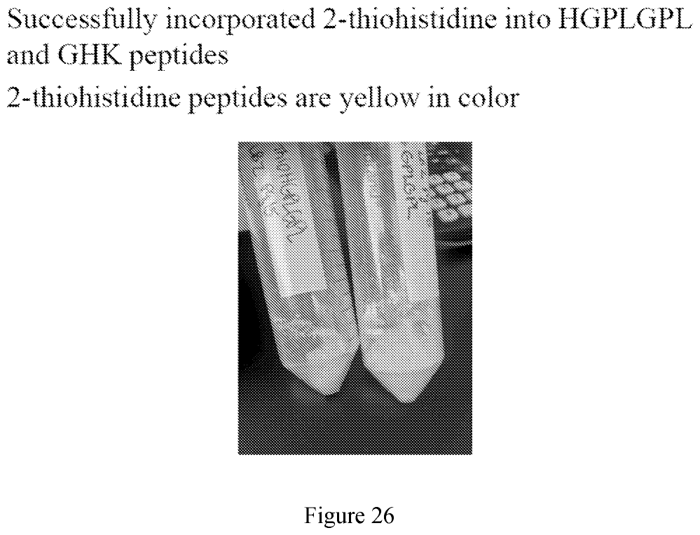

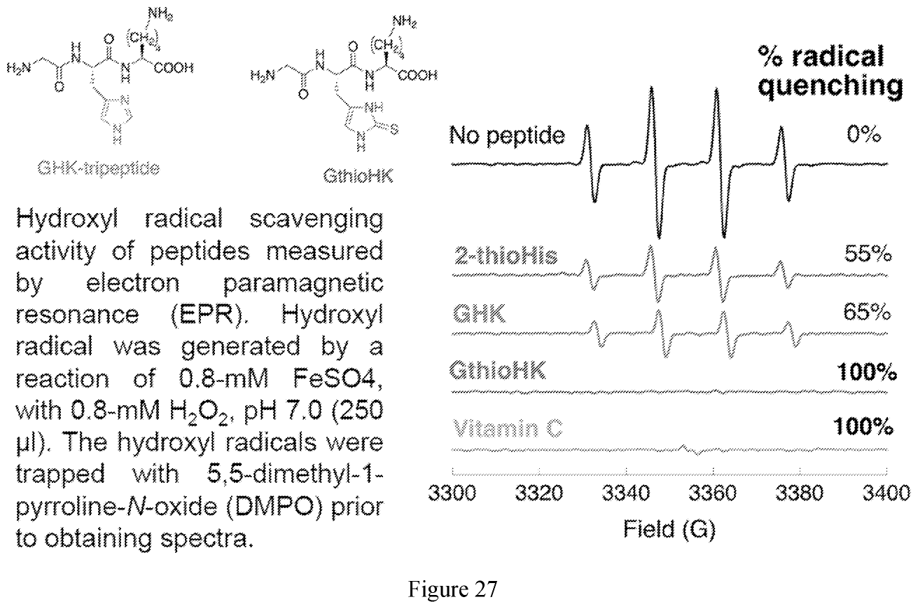

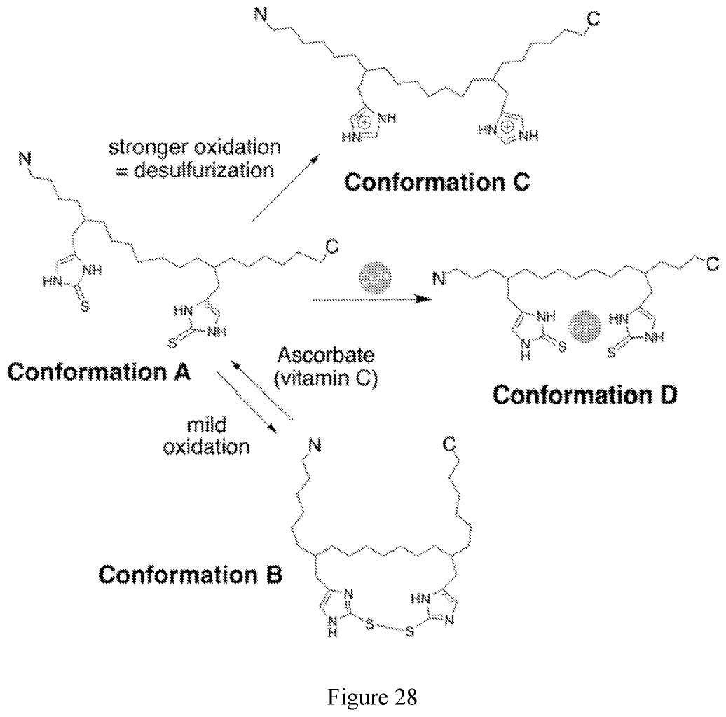

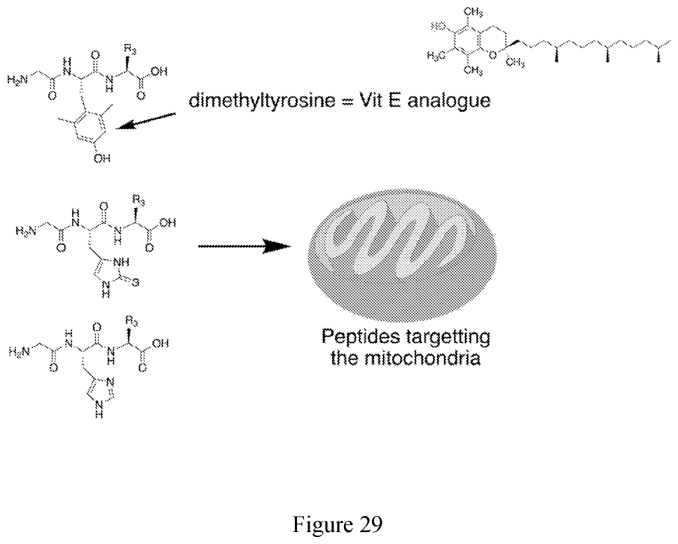

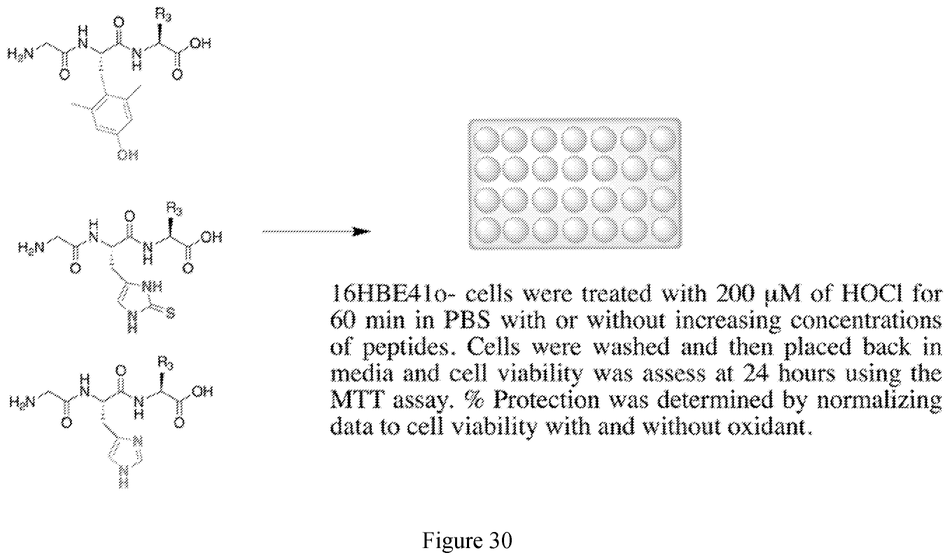

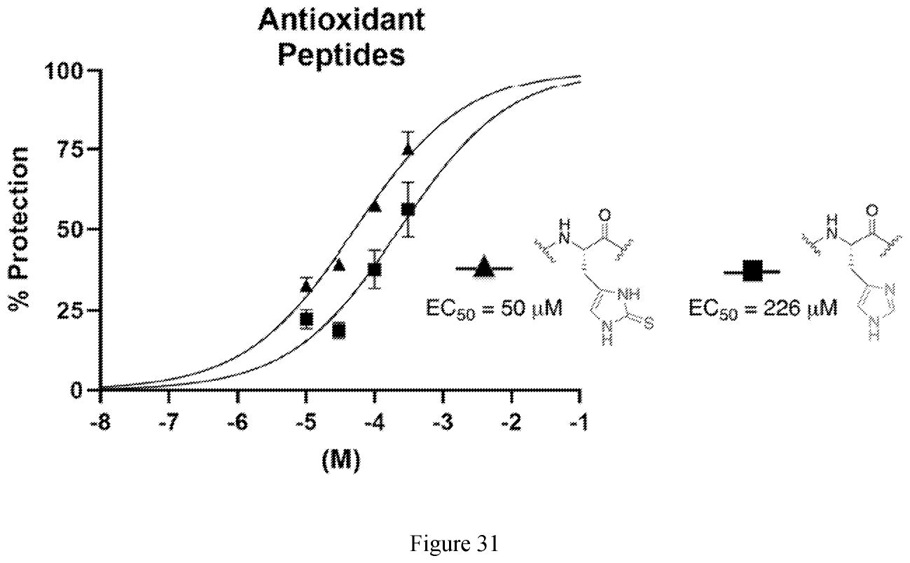

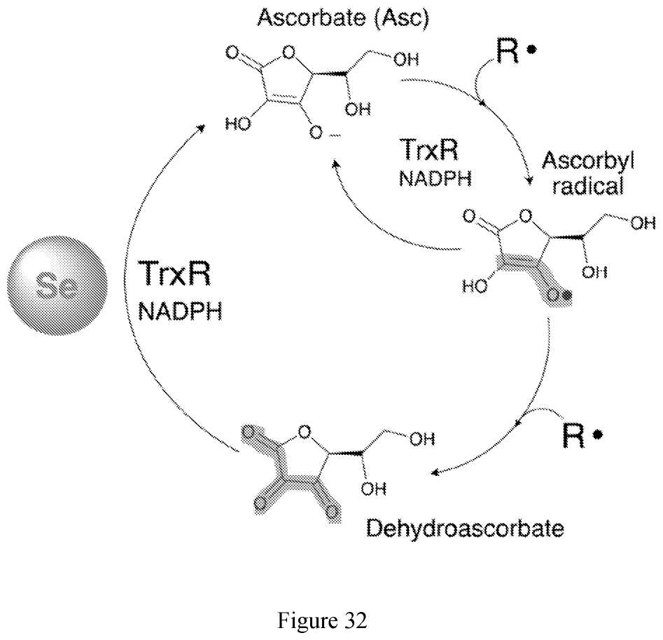

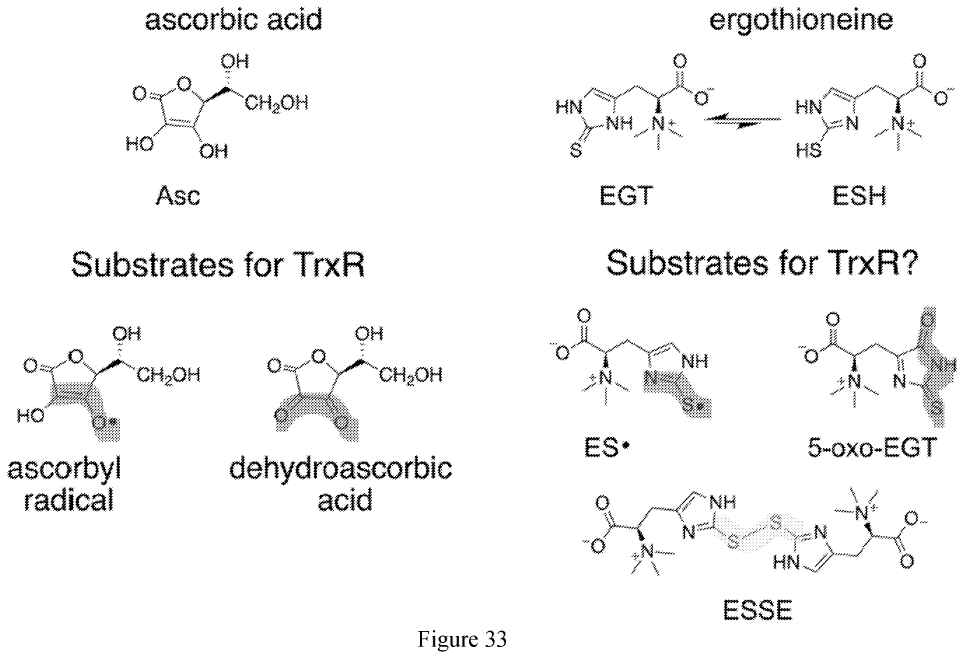

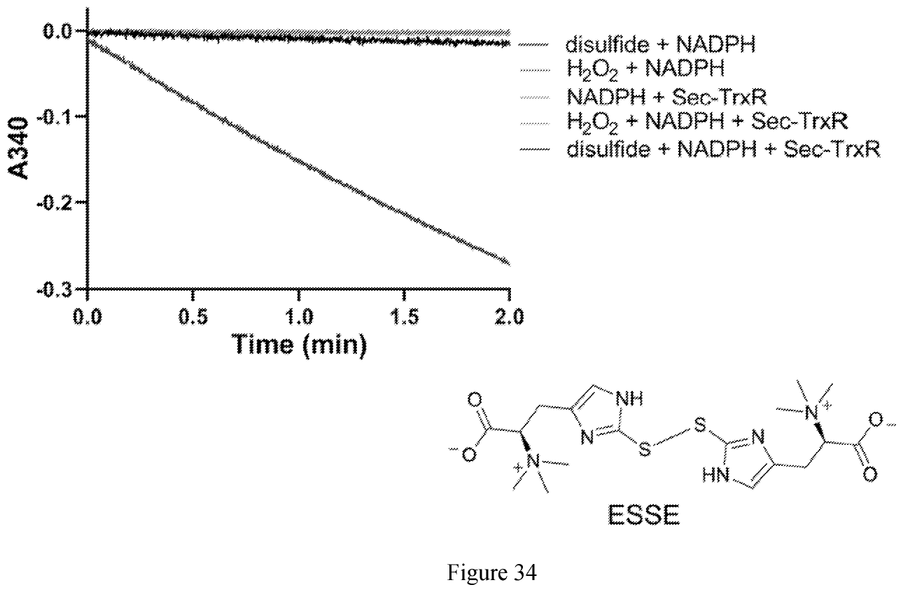

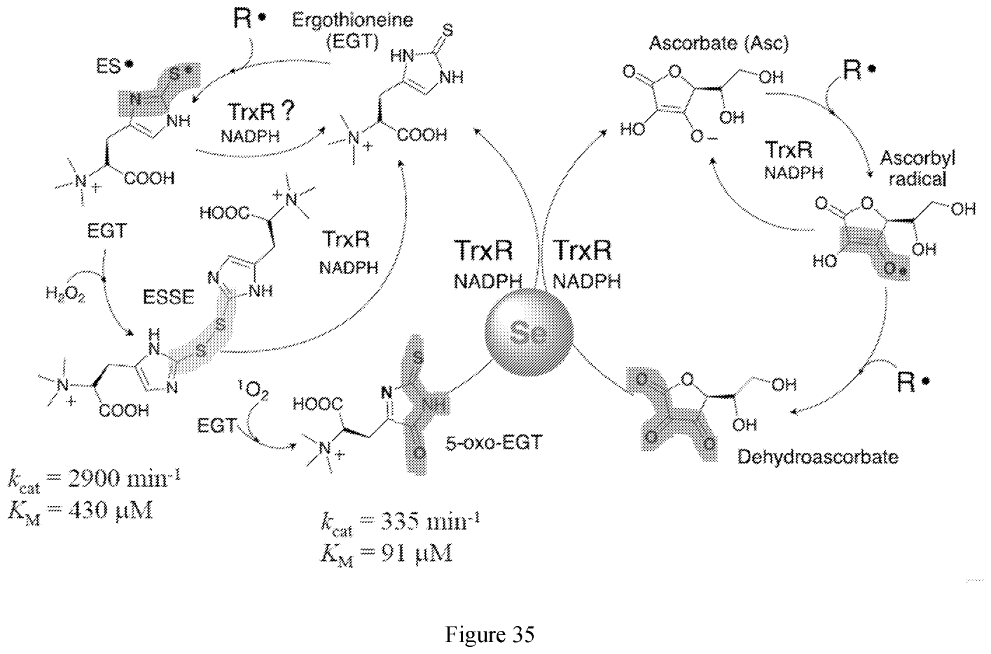

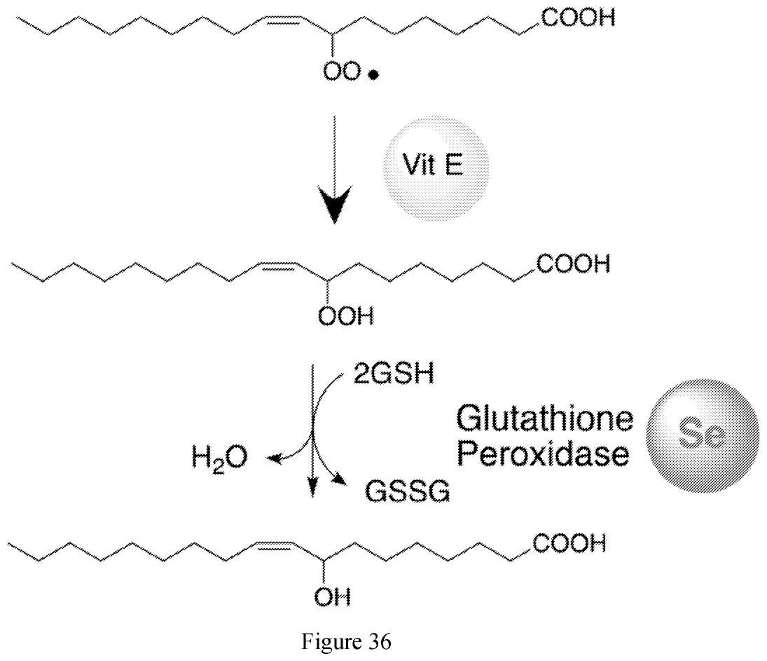

Cell viability assays were also performed. They were performed on 96-well plates (8 rows×12 columns). All CVAs were performed on Human Mesothelioma Cells. After incubating for at least 24 h at 37° C. with peptide and rotenone, live cells stick to the bottom surface while dead cells can be expelled. After removing the dead cells, the remaining live cells in each well are stained with crystal violet. After staining with crystal violet, there were two ways used to determine % survival: Cell Count: a Lionheart plate reader counted the number of stained cells in each well. % Cell survival was determined by comparison to a control of 0 μM peptide and/or 0 μM rotenone. A₅₄₀ Measurement: After cell count, MeOH was used to dissolve the crystal violet in each well, and absorbance of 540 nm light for each well was compared to a control of 0 μM peptide and/or 0 μM rotenone. The relative A₅₄₀ reading thus gave a rough indication of cell survival. Wells with greater survival would have a greater A₅₄₀, as the violet stain is fixed to the live cells. These data are shown in FIGS. 10-15.

Although the present disclosure has been described with respect to one or more particular embodiments and/or examples, it will be understood that other embodiments and/or examples of the present disclosure may be made without departing from the scope of the present disclosure.

1. A peptide or protein comprising the following sequence: -RXKF-, wherein X is a 2TH residue or a 2TH analogue residue and each residue of -RXKF- has D or L stereochemistry.

2. The peptide or protein of claim 1, wherein the 2TH analogue residue has the following structure:

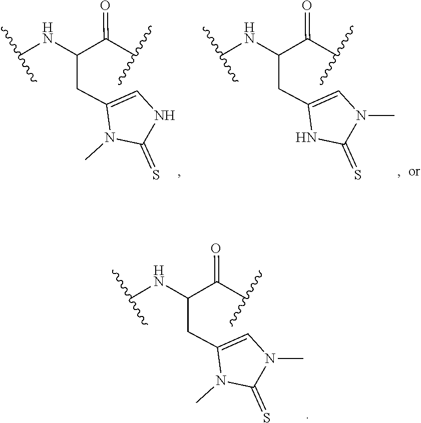 [SMILES: *NC(Cc1c[nH]c(=S)n1C)C(*)=O ; *NC(Cc1cn(C)c(=S)[nH]1)C(*)=O ; *NC(Cc1cn(C)c(=S)n1C)C(*)=O]

3. The peptide or protein according to claim 1, wherein the peptide or protein comprises the following sequence: 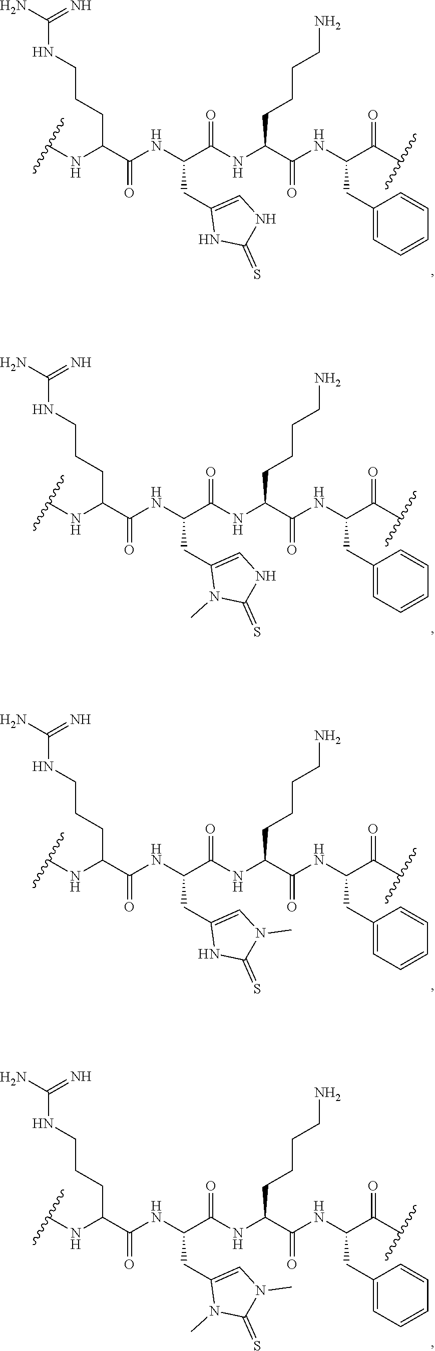 [SMILES: *NC(CCCNC(=N)N)C(=O)N[C@@H](Cc1c[nH]c(=S)[nH]1)C(=O)N[C@@H](CCCCN)C(=O)N[C@@H](Cc1ccccc1)C(*)=O ; *NC(CCCNC(=N)N)C(=O)N[C@@H](Cc1c[nH]c(=S)n1C)C(=O)N[C@@H](CCCCN)C(=O)N[C@@H](Cc1ccccc1)C(*)=O ; *NC(CCCNC(=N)N)C(=O)N[C@@H](Cc1cn(C)c(=S)[nH]1)C(=O)N[C@@H](CCCCN)C(=O)N[C@@H](Cc1ccccc1)C(*)=O ; *NC(CCCNC(=N)N)C(=O)N[C@@H](Cc1cn(C)c(=S)n1C)C(=O)N[C@@H](CCCCN)C(=O)N[C@@H](Cc1ccccc1)C(*)=O]
or a protonated analogue thereof.

4. The peptide or protein according to claim 1, wherein the peptide or protein comprises the following sequence: 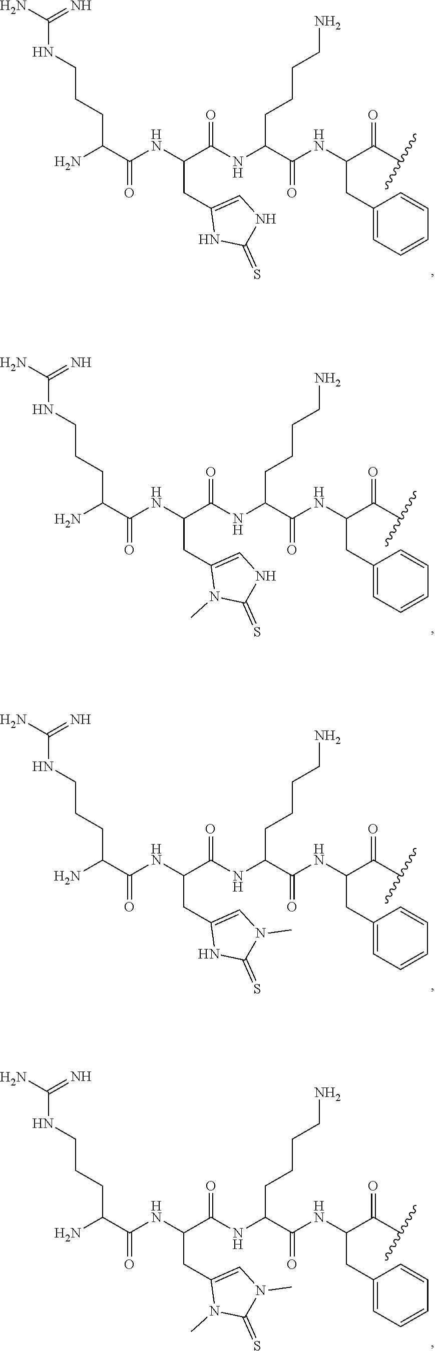 [SMILES: *C(=O)C(Cc1ccccc1)NC(=O)C(CCCCN)NC(=O)C(Cc1c[nH]c(=S)[nH]1)NC(=O)C(N)CCCNC(=N)N ; *C(=O)C(Cc1ccccc1)NC(=O)C(CCCCN)NC(=O)C(Cc1c[nH]c(=S)n1C)NC(=O)C(N)CCCNC(=N)N ; *C(=O)C(Cc1ccccc1)NC(=O)C(CCCCN)NC(=O)C(Cc1cn(C)c(=S)[nH]1)NC(=O)C(N)CCCNC(=N)N ; *C(=O)C(Cc1ccccc1)NC(=O)C(CCCCN)NC(=O)C(Cc1cn(C)c(=S)n1C)NC(=O)C(N)CCCNC(=N)N]
or a protonated analogue thereof.

5. The peptide or protein according to the claim 4, wherein the peptide or protein comprises the following sequence: 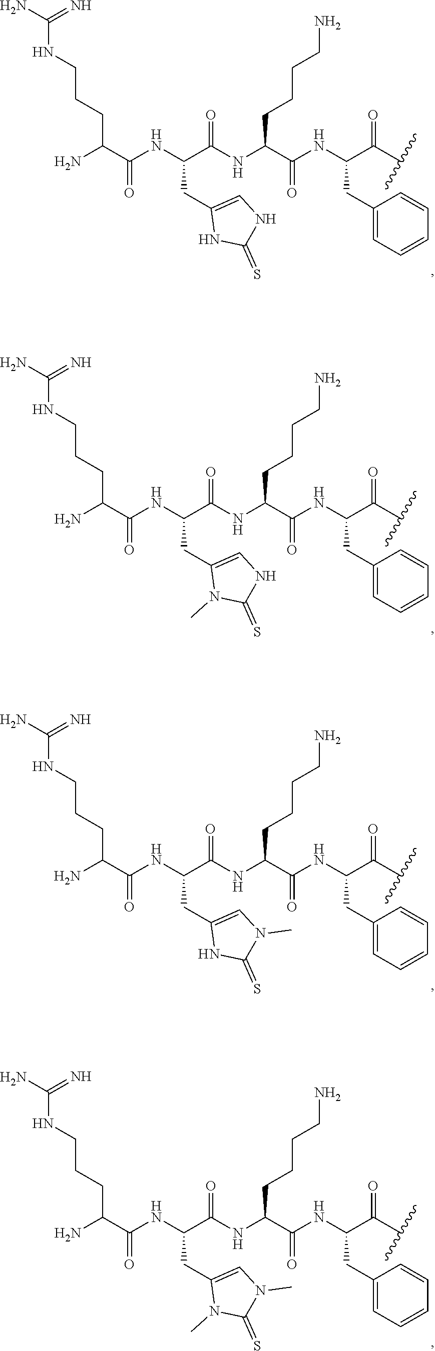 [SMILES: *C(=O)[C@H](Cc1ccccc1)NC(=O)[C@H](CCCCN)NC(=O)[C@H](Cc1c[nH]c(=S)[nH]1)NC(=O)C(N)CCCNC(=N)N ; *C(=O)[C@H](Cc1ccccc1)NC(=O)[C@H](CCCCN)NC(=O)[C@H](Cc1c[nH]c(=S)n1C)NC(=O)C(N)CCCNC(=N)N ; *C(=O)[C@H](Cc1ccccc1)NC(=O)[C@H](CCCCN)NC(=O)[C@H](Cc1cn(C)c(=S)[nH]1)NC(=O)C(N)CCCNC(=N)N ; *C(=O)[C@H](Cc1ccccc1)NC(=O)[C@H](CCCCN)NC(=O)[C@H](Cc1cn(C)c(=S)n1C)NC(=O)C(N)CCCNC(=N)N]
or a protonated analogue thereof.

6. The peptide or protein according to claim 1, wherein the peptide or protein comprises the following sequence: 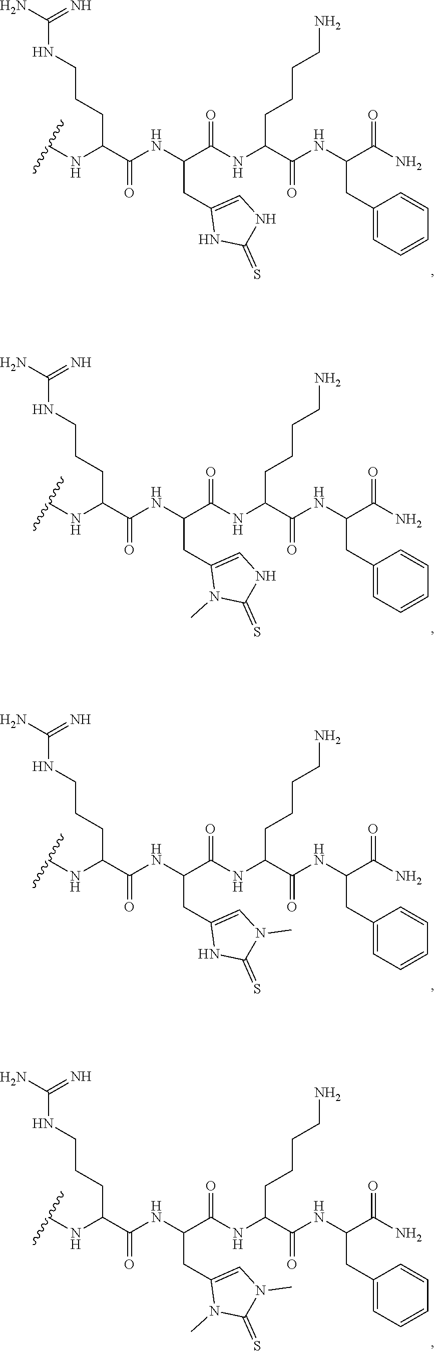 [SMILES: *NC(CCCNC(=N)N)C(=O)NC(Cc1c[nH]c(=S)[nH]1)C(=O)NC(CCCCN)C(=O)NC(Cc1ccccc1)C(N)=O ; *NC(CCCNC(=N)N)C(=O)NC(Cc1c[nH]c(=S)n1C)C(=O)NC(CCCCN)C(=O)NC(Cc1ccccc1)C(N)=O ; *NC(CCCNC(=N)N)C(=O)NC(Cc1cn(C)c(=S)[nH]1)C(=O)NC(CCCCN)C(=O)NC(Cc1ccccc1)C(N)=O ; *NC(CCCNC(=N)N)C(=O)NC(Cc1cn(C)c(=S)n1C)C(=O)NC(CCCCN)C(=O)NC(Cc1ccccc1)C(N)=O]
or a protonated analogue thereof.

7. The peptide or protein according to claim 6, wherein the peptide or protein comprises the following sequence: 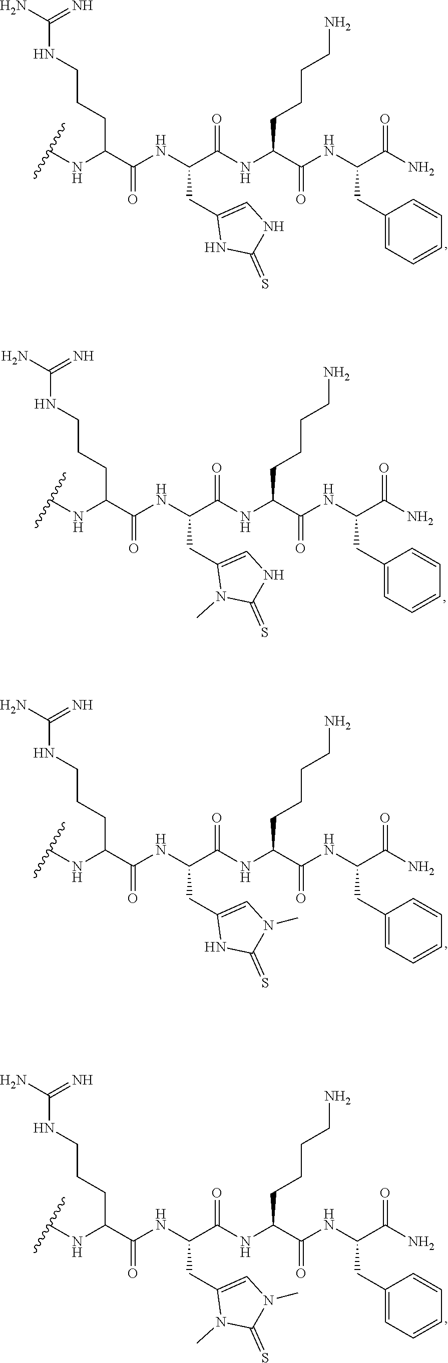 [SMILES: *NC(CCCNC(=N)N)C(=O)N[C@@H](Cc1c[nH]c(=S)[nH]1)C(=O)N[C@@H](CCCCN)C(=O)N[C@@H](Cc1ccccc1)C(N)=O ; *NC(CCCNC(=N)N)C(=O)N[C@@H](Cc1c[nH]c(=S)n1C)C(=O)N[C@@H](CCCCN)C(=O)N[C@@H](Cc1ccccc1)C(N)=O ; *NC(CCCNC(=N)N)C(=O)N[C@@H](Cc1cn(C)c(=S)[nH]1)C(=O)N[C@@H](CCCCN)C(=O)N[C@@H](Cc1ccccc1)C(N)=O ; *NC(CCCNC(=N)N)C(=O)N[C@@H](Cc1cn(C)c(=S)n1C)C(=O)N[C@@H](CCCCN)C(=O)N[C@@H](Cc1ccccc1)C(N)=O]
or a protonated analogue thereof.

8. The peptide or protein according to claim 1, wherein the peptide or protein comprises the following sequence: 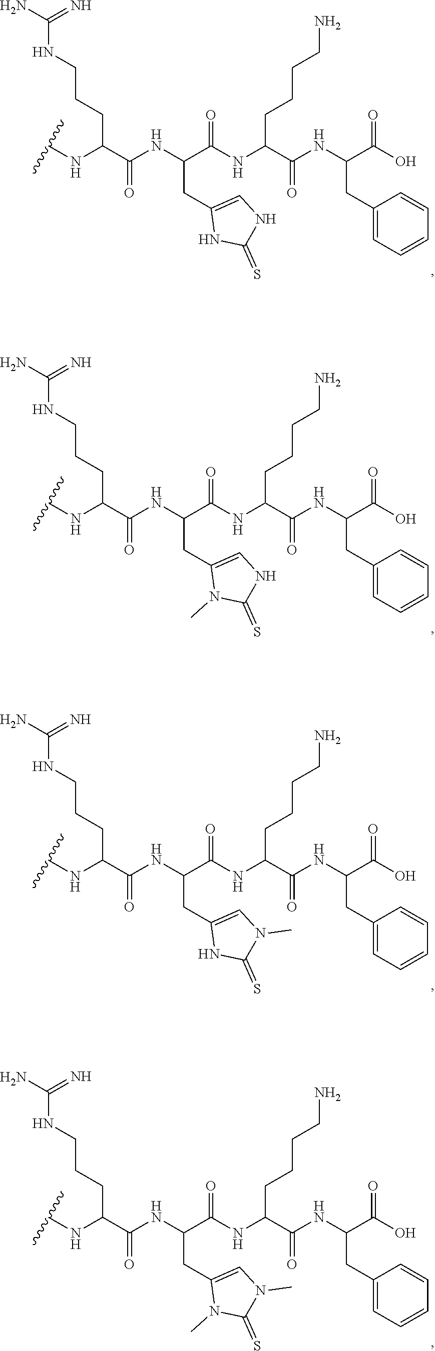 [SMILES: *NC(CCCNC(=N)N)C(=O)NC(Cc1c[nH]c(=S)[nH]1)C(=O)NC(CCCCN)C(=O)NC(Cc1ccccc1)C(=O)O ; *NC(CCCNC(=N)N)C(=O)NC(Cc1c[nH]c(=S)n1C)C(=O)NC(CCCCN)C(=O)NC(Cc1ccccc1)C(=O)O ; *NC(CCCNC(=N)N)C(=O)NC(Cc1cn(C)c(=S)[nH]1)C(=O)NC(CCCCN)C(=O)NC(Cc1ccccc1)C(=O)O ; *NC(CCCNC(=N)N)C(=O)NC(Cc1cn(C)c(=S)n1C)C(=O)NC(CCCCN)C(=O)NC(Cc1ccccc1)C(=O)O]
or a protonated or deprotonated analogue thereof.

9. A peptide or protein according to claim 8, wherein the peptide or protein comprises the following sequence: 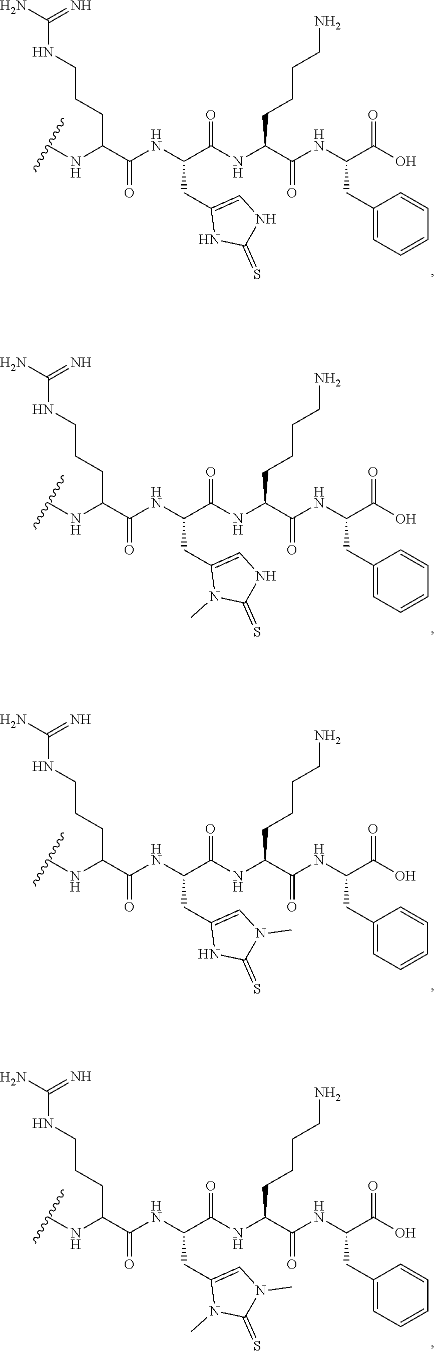 [SMILES: *NC(CCCNC(=N)N)C(=O)N[C@@H](Cc1c[nH]c(=S)[nH]1)C(=O)N[C@@H](CCCCN)C(=O)N[C@@H](Cc1ccccc1)C(=O)O ; *NC(CCCNC(=N)N)C(=O)N[C@@H](Cc1c[nH]c(=S)n1C)C(=O)N[C@@H](CCCCN)C(=O)N[C@@H](Cc1ccccc1)C(=O)O ; *NC(CCCNC(=N)N)C(=O)N[C@@H](Cc1cn(C)c(=S)[nH]1)C(=O)N[C@@H](CCCCN)C(=O)N[C@@H](Cc1ccccc1)C(=O)O ; *NC(CCCNC(=N)N)C(=O)N[C@@H](Cc1cn(C)c(=S)n1C)C(=O)N[C@@H](CCCCN)C(=O)N[C@@H](Cc1ccccc1)C(=O)O]
or a protonated or deprotonated analogue thereof.

10. A peptide or protein according to claim 1, wherein the peptide or protein has the following sequence: 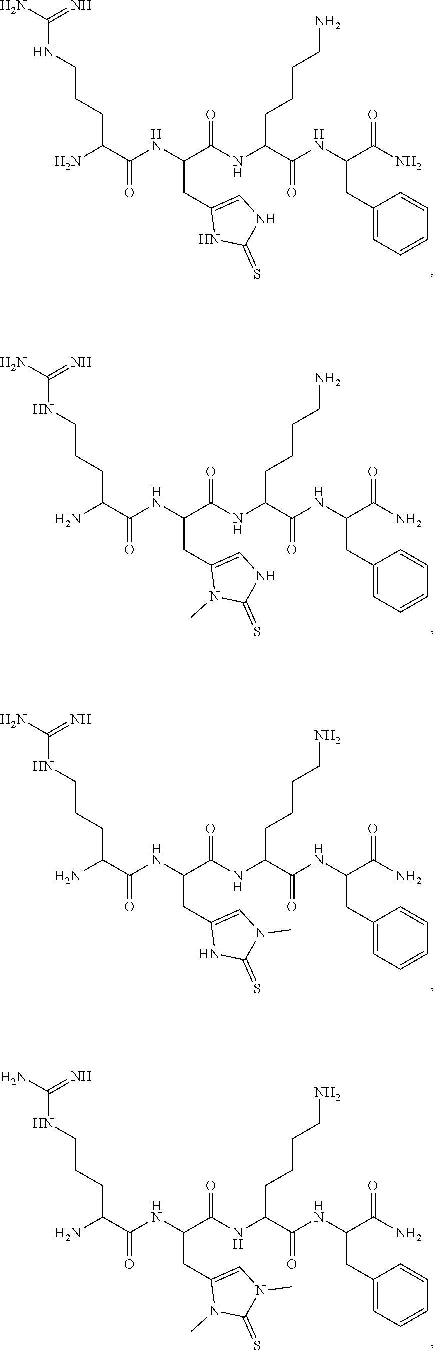 [SMILES: N=C(N)NCCCC(N)C(=O)NC(Cc1c[nH]c(=S)[nH]1)C(=O)NC(CCCCN)C(=O)NC(Cc1ccccc1)C(N)=O ; Cn1c(CC(NC(=O)C(N)CCCNC(=N)N)C(=O)NC(CCCCN)C(=O)NC(Cc2ccccc2)C(N)=O)c[nH]c1=S ; Cn1cc(CC(NC(=O)C(N)CCCNC(=N)N)C(=O)NC(CCCCN)C(=O)NC(Cc2ccccc2)C(N)=O)[nH]c1=S ; Cn1cc(CC(NC(=O)C(N)CCCNC(=N)N)C(=O)NC(CCCCN)C(=O)NC(Cc2ccccc2)C(N)=O)n(C)c1=S]
or a protonated analogue thereof.

11. A peptide or protein according to claim 9, wherein the peptide or protein has the following sequence: 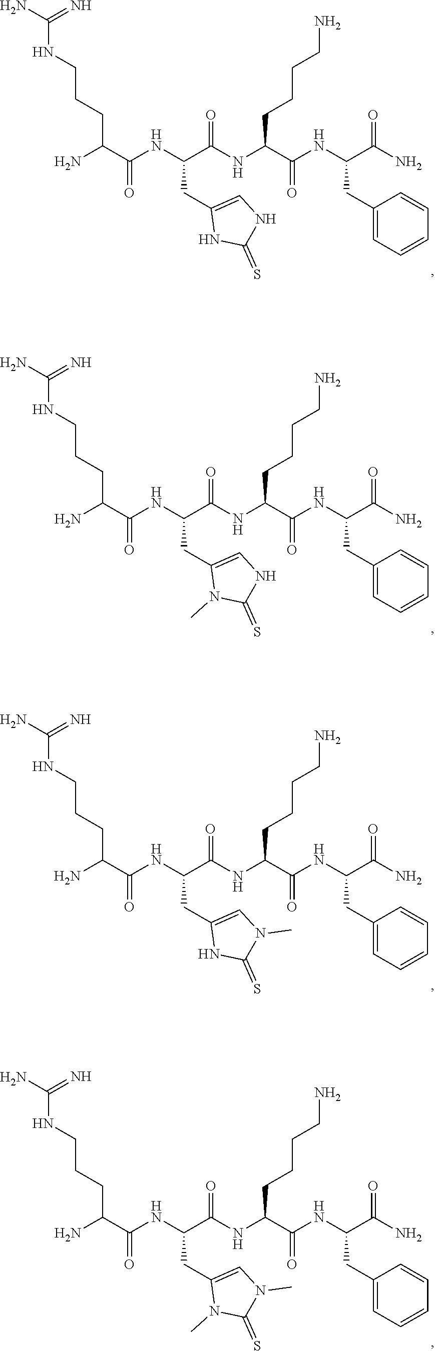 [SMILES: N=C(N)NCCCC(N)C(=O)N[C@@H](Cc1c[nH]c(=S)[nH]1)C(=O)N[C@@H](CCCCN)C(=O)N[C@@H](Cc1ccccc1)C(N)=O ; Cn1c(C[C@H](NC(=O)C(N)CCCNC(=N)N)C(=O)N[C@@H](CCCCN)C(=O)N[C@@H](Cc2ccccc2)C(N)=O)c[nH]c1=S ; Cn1cc(C[C@H](NC(=O)C(N)CCCNC(=N)N)C(=O)N[C@@H](CCCCN)C(=O)N[C@@H](Cc2ccccc2)C(N)=O)[nH]c1=S ; Cn1cc(C[C@H](NC(=O)C(N)CCCNC(=N)N)C(=O)N[C@@H](CCCCN)C(=O)N[C@@H](Cc2ccccc2)C(N)=O)n(C)c1=S]
or a protonated analogue thereof.

12. The peptide or protein according to claim 1, wherein the peptide or protein has the following sequence: 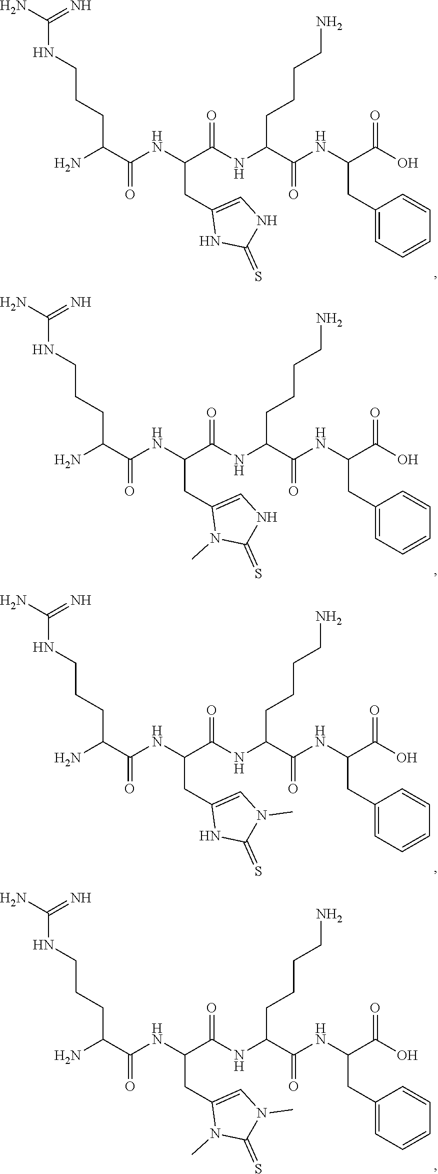 [SMILES: N=C(N)NCCCC(N)C(=O)NC(Cc1c[nH]c(=S)[nH]1)C(=O)NC(CCCCN)C(=O)NC(Cc1ccccc1)C(=O)O ; Cn1c(CC(NC(=O)C(N)CCCNC(=N)N)C(=O)NC(CCCCN)C(=O)NC(Cc2ccccc2)C(=O)O)c[nH]c1=S ; Cn1cc(CC(NC(=O)C(N)CCCNC(=N)N)C(=O)NC(CCCCN)C(=O)NC(Cc2ccccc2)C(=O)O)[nH]c1=S ; Cn1cc(CC(NC(=O)C(N)CCCNC(=N)N)C(=O)NC(CCCCN)C(=O)NC(Cc2ccccc2)C(=O)O)n(C)c1=S]
or a protonated or deprotonated analogue thereof.

13. The peptide or protein according to claim 12, wherein the peptide or protein has the following sequence: 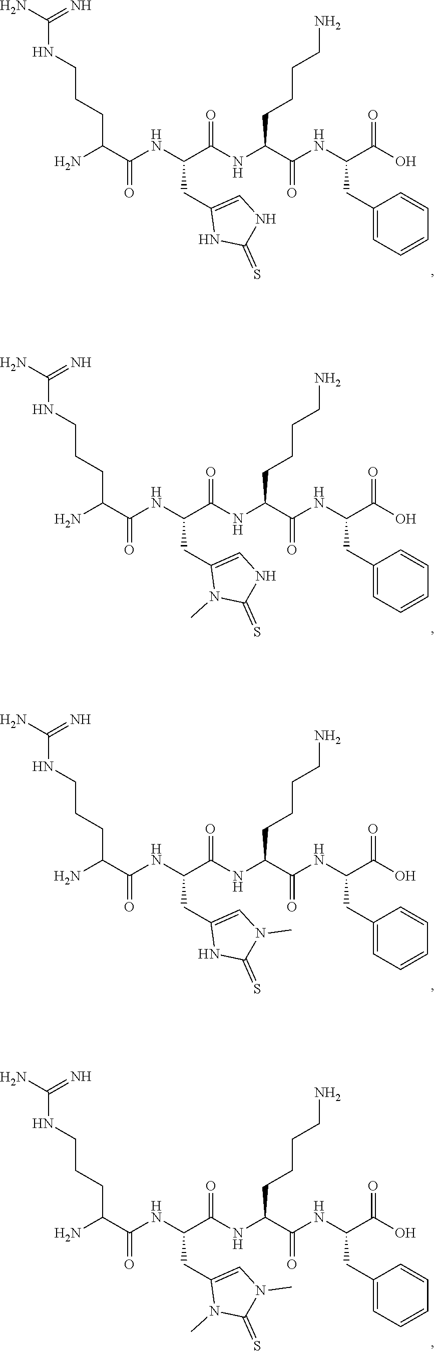 [SMILES: N=C(N)NCCCC(N)C(=O)N[C@@H](Cc1c[nH]c(=S)[nH]1)C(=O)N[C@@H](CCCCN)C(=O)N[C@@H](Cc1ccccc1)C(=O)O ; Cn1c(C[C@H](NC(=O)C(N)CCCNC(=N)N)C(=O)N[C@@H](CCCCN)C(=O)N[C@@H](Cc2ccccc2)C(=O)O)c[nH]c1=S ; Cn1cc(C[C@H](NC(=O)C(N)CCCNC(=N)N)C(=O)N[C@@H](CCCCN)C(=O)N[C@@H](Cc2ccccc2)C(=O)O)[nH]c1=S ; Cn1cc(C[C@H](NC(=O)C(N)CCCNC(=N)N)C(=O)N[C@@H](CCCCN)C(=O)N[C@@H](Cc2ccccc2)C(=O)O)n(C)c1=S]
or a protonated or deprotonated analogue thereof.

14. The peptide or protein according to claim 1, wherein the peptide has the following structure: 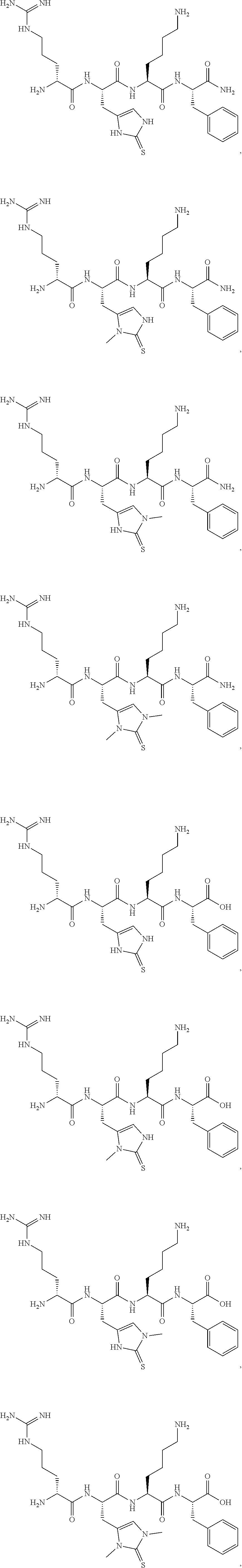
or a protonated or deprotonated analogue thereof.

15. A composition comprising a peptide or protein according to claim 1.

16. The composition according to claim 15, wherein the composition further comprises a pharmaceutically acceptable carrier.

17. A method for treating an individual having or suspected of having a disease associated with mitochondrial dysfunction or an individual having or suspected of having chlorine gas exposure, comprising administering to the individual a composition according to claim 15.

18. The method according to claim 17, wherein the disease is Duchenne's muscular dystrophy, age-related macular degeneration, Barth syndrome, Leber's hereditary optic neuropathy, or a combination thereof.

19. A kit comprising a peptide or protein according to claim 1 and instructions for use.

20. The kit according to claim 19, wherein the kit further comprises a diluent or pharmaceutically acceptable carrier.

21. The kit according to claim 19, wherein the peptide or protein is provided as a lyophilized powder.

22. The kit according to claim 19, wherein the peptide or protein is provided dissolved a solvent.


SEQUENCE LISTING (2 sequences)

SEQ ID NO: 1 - protein, 4 aa, synthetic construct
RHKF

SEQ ID NO: 2 - protein, 7 aa, synthetic construct
HGPLGPL